SEQUENCE LISTING (7 sequences)

SEQ ID NO: 1 - DNA, 37 bp, Artificial
  Forward CCDC80 PCR Primer
acgctgtcgaccaccgcaaccctctgcattccatctc

SEQ ID NO: 2 - DNA, 61 bp, Artificial
  Reverse CCDC80 PCR Primer
cgtctagattcacttatcgtcgtcatccttgtaatcgtaagggtatccatggtgataactc

SEQ ID NO: 3 - DNA, 97 bp, Artificial
  DNA Encoding Mouse CCDC80 ShRNA
tgctgttgacagtgagcgccctgagaaggagaagaagaaatagtgaagccacagatgtatttcttcttctccttctcaggttgcctactgcctcgga

SEQ ID NO: 4 - DNA, 119 bp, Artificial
  DNA Encoding Non-Silencing ShRNA
atctcgcttgggcgagagtaagtgctgttgacagtgagcgatctcgcttgggcgagagtaagtagtgaagccacagatgtacttactctcgcccaagcgagagtgcctactgcctcgga

SEQ ID NO: 5 - protein, 18 aa, Artificial
  Synthetic Peptide Corresponding to Amino Acids 148-165 of Human and Mouse CCDC80
KNRVWVISAPHASEGYYR

SEQ ID NO: 6 - protein, 14 aa, Artificial
  Synthetic Peptide Corresponding to Amino Acids 672-685 of Human CCDC80 and 671-684 of Mouse CCDC80
KIDHFQLDNEKPMR

SEQ ID NO: 7 - RNA, 97 nt, Artificial
  Mouse CCDC80 shRNA
ugcuguugacagugagcgcccugagaaggagaagaagaaauagugaagccacagauguauuucuucuucuccuucucagguugccuacugccucgga

(19) United States
(12) Patent Application Publication
(10) Pub. No.: US 2008/0221057 A1
(43) Pub. Date: Sep. 11, 2008

(54) SECRETED PROTEIN CCDC80 REGULATES ADIPOCYTE DIFFERENTIATION

(75) Inventors: Ruth Elisabeth Gimeno, Wellesley, MA (US); Frederic Tremblay, Cambridge, MA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/030,347

(22) Filed: Feb. 13, 2008

Related U.S. Application Data
(60) Provisional application No. 60/901,882, filed on Feb. 16, 2007.
(60) Provisional application No. 60/997,920, filed on Oct. 5, 2007.

(51) Int. Cl. A61K 31/70; C12N 5/06; C12Q 1/02; A61P 1/00

(52) U.S. Cl. 514/44; 435/375; 435/29

(57) ABSTRACT

Disclosed herein are methods of modulating adipogenesis. The methods include contacting a cell expressing the Ccdc80 gene with an agent that modulates the expression or activity of the Ccdc80 gene or Ccdc80 protein. Further disclosed herein are methods of treating conditions such as obesity, insulin resistance, and/or type 2 diabetes with Ccdc80 modulators. Also disclosed herein are methods of identifying Ccdc80 modulators.

### CROSS-REFERENCE TO RELATED APPLICATIONS

This application claims the benefit of U.S. Provisional Application No. 60/901,882, filed Feb. 16, 2007 and U.S. Provisional Application No. 60/997,920, filed Oct. 5, 2007, the contents each of which are incorporated herein by reference in their entirety.

### FIELD OF THE INVENTION

The present invention relates to methods of modulating adipogenesis in a cell. In particular, the invention relates to the use of an agent that modulates the expression or activity of the Ccdc80 gene or Ccdc80 protein.

### BACKGROUND OF THE INVENTION

Adipose tissue is increasingly recognized as an active endocrine organ that secretes a variety of factors, collectively named “adipokines” (Gimeno R E & Klaman L D, Curr. Opin. Pharmacol. 5:122-28 (2005); Kershaw E E & Flier J S, J. Clin. Endocrinol. Metab. 89:2548-56 (2004)). Known adipokines include metabolic mediators such as leptin, adiponectin, and resistin; regulators of thrombosis such as PAI-1; and inflammatory mediators such as TNFα. Adipokines act in an endocrine or paracrine manner on a variety of target tissues, including muscle, liver, brain, and bone. Adipokines affect energy homeostasis (e.g., leptin), insulin sensitivity (e.g., adiponectin), vascular function (e.g., PAI-1), and bone metabolism (Gimeno R E & Klaman L D, supra; Khosla S, Endocrinology 143:4161-64 (2002); Fu L et al., Cell 122:803-15 (2005); Oshima K et al., Biochem. Biophys. Res. Commun. 331:520-26 (2005); Takeda S et al., Annu. Rev. Nutr. 23:403-11 (2003)). The identification of additional adipokines and the characterization of their effects on different target tissues are therefore an area of intense investigation.

Ccdc80 (also termed mouse URB (up-regulated in bombesin receptor subtype-3 knockout mice), human DRO1 (down-regulated by oncogenes 1), rat SSG1 (steroid-sensitive gene 1), chicken EQUARIN) was initially described as a ubiquitously expressed gene that is up-regulated in the brown adipose tissue of bombesin receptor subtype-3 knock-out mice (Aoki K et al., Biochem. Biophys. Res. Commun. 290:1282-88 (2002)). Subsequently, human Ccdc80 was shown to be expressed in bone marrow stromal cells and to be down-regulated during differentiation of these cells into osteoblasts (Liu Y et al., Biochem. Biophys. Res. Commun. 322:497-507 (2004)). Ccdc80 mRNA and protein were also shown to be present in chondrocytes and associated extracellular matrix during mouse embryo development (Liu Y et al., supra). A chicken ortholog of Ccdc80 was found to be expressed exclusively in the lens equatorial region (Mu H et al., Mech. Dev. 120:143-55 (2003)). While mouse Ccdc80 and chicken Ccdc80 have been demonstrated to be secreted proteins (Liu Y et al., supra; Mu H et al., supra), human Ccdc80 was reported to be localized intracellularly with no appreciable amounts being secreted (Bommer G T et al., J. Biol. Chem. 280:7962-75 (2005)). Human Ccdc80 was found to be down-regulated in cells neoplastically transformed with β-catenin, and overexpression of Ccdc80 in these cells was able to inhibit growth, leading to the designation of Ccdc80 as a candidate tumor suppressor gene (Bommer G T et al., supra). To date, little or no other functional data have been reported for any mammalian Ccdc80 orthologs.

### SUMMARY OF THE INVENTION

The present invention provides a method of modulating adipogenesis in a cell. The method of the present invention has applications in therapeutic, prophylactic and cosmetic treatments. The method of modulating adipogenesis in a cell includes contacting the cell with an agent that modulates the expression or activity of the Ccdc80 gene or Ccdc80 protein. The cell may be an adipocytic cell, such as a pre-adipocyte, adipocyte, mesenchymal stem cell, embryonic stem cell or embryonic fibroblast. The agent may be a compound, a protein, a peptide, an antibody, an aptamer, or a polynucleotide. In some embodiments, such agents may directly modulate the expression or ability of the Cddc80 gene or Ccdc80 protein. In some embodiments, the agent increases Ccdc80 gene expression or Ccdc80 protein expression or activity. In some other embodiments, the agent prevents or reduces Ccdc80 gene expression or Ccdc80 protein expression or activity.

In some embodiments, the method of modulating adipogenesis involves contacting a cell with an agent that prevents or reduces at least one of Ccdc80 gene transcription or translation of Ccdc80 messenger ribonucleic acid (mRNA). The agent may be a polynucleotide. In some embodiments, the polynucleotide is ribonucleic acid (RNA). In certain embodiments, the polynucleotide may be a Ccdc80 antisense polynucleotide. The polynucleotide may, for example, be a dsRNA, a ribozyme, or an antisense oligonucleotide. In some embodiments, the polynucleotide may be an shRNA or a siRNA. In certain embodiments, a polynucleotide agent that prevents or reduces translation of Ccdc80 mRNA is an shRNA. In some embodiments, the shRNA includes a nucleic acid sequence that hybridizes under high stringency conditions to a Ccdc80 gene sequence of SEQ ID NO: 3. The shRNA may include the nucleic acid sequence of SEQ ID NO: 7, for example. In some embodiments, a nucleic acid sequence that hybridizes under high stringency conditions to a Ccdc80 gene sequence of SEQ ID NO: 3 is at least 85%, 90%, 95% or more identical to SEQ ID NO: 7.

As described above, a polynucleotide that prevents or reduces at least one of Ccdc80 gene transcription or translation of Ccdc80 mRNA may be RNA. Alternatively, such a polynucleotide may be deoxyribonucleic acid (DNA). A polynucleotide may be linked to a peptide or antibody, which binds to at least one cell surface receptor or antigen of the cell.

In some embodiments, the agent prevents or reduces the activity of Ccdc80 protein. For example, in some embodiments, the agent is an antibody against Ccdc80 protein.

In some further embodiments, an agent that modulates the expression or activity of the Ccdc80 gene or Ccdc80 protein may be a nucleic acid encoding a Ccdc80 polypeptide.

In some embodiments, a method of modulating adipogenesis in a cell that expresses the Ccdc80 gene involves modulating Wnt/β-catenin signaling. In some embodiments, modulating Wnt/β-catenin signaling involves administering to a cell an agent that modulates the expression or activity of the CCDc80 gene or Ccdc80 protein. The agent may, for example, be a compound, a protein, a peptide, an antibody, an aptamer, or a polynucleotide. In some embodiments, such agents directly modulate this expression or activity of the Ccdc80 gene or Ccdc80 protein. A polynucleotide that modulates the expression or activity of the Ccdc80 gene or Ccdc80 protein, thereby modulating Wnt/β-catenin signaling, may be an shRNA, such as an shRNA including the nucleic acid sequence of SEQ ID NO: 7. In some embodiments, a nucleic acid sequence that modulates the expression or activity of the Ccdc80 gene or Ccdc80 protein is at least 85%, 90%, 95% or more identical to SEQ ID NO: 7.

The present invention further provides a method of treating a condition selected from obesity, insulin resistance, or type 2 diabetes. The method includes administering to a subject in need thereof an agent that modulates the expression or activity of the Ccdc80 gene or Ccdc80 protein. In some embodiments, the agent directly modulates the expression or activity of the Ccdc80 gene or Ccdc80 protein. In some embodiments, the condition treated is obesity.

Obesity is defined herein as a body weight disorder. In some embodiments, obesity may be defined as a condition describing excess body weight in the form of fat. In addition to providing a therapeutic method of treating obesity, the present invention also provides a cosmetic method of treating obesity. The cosmetic treatment method includes administering to a subject having excess body weight in the form of fat an agent that modulates the expression or activity of the Ccdc80 gene or Ccdc80 protein. Also provided is the use of an agent that modulates the expression or activity of the Ccdc80 gene or Ccdc80 protein as a cosmetic product for reducing excess body weight in the form of fat. Further provided is a composition for cosmetic treatment of obesity, the composition comprising an agent that modulates the expression or activity of the Ccdc80 gene or Ccdc80 protein.

In some embodiments, the agent administered to treat obesity increases Ccdc80 gene expression or Ccdc80 protein expression or activity. In some other embodiments, the agent administered to treat obesity prevents or reduces Ccdc80 gene expression or Ccdc80 protein expression or activity. The agent used to treat obesity may be an agent that prevents or reduces Ccdc80 gene transcription. Alternatively, the agent used to treat obesity may be an agent that prevents or reduces translation of Ccdc80 mRNA.

An administered anti-obesity agent that prevents or reduces at least one of Ccdc80 gene transcription or translation of Ccdc80 mRNA may be a polynucleotide. In some embodiments, this polynucleotide is RNA. The administered anti-obesity RNA may be, for example, a Ccdc80 antisense polynucleotide, such as a double stranded RNA (dsRNA), a ribozyme, or an antisense oligonucleotide. In some other embodiments, the administered anti-obesity RNA is a short hairpin RNA (shRNA) or a small interfering RNA (siRNA).

In some embodiments, an administered anti-obesity agent is a short hairpin RNA (shRNA). In some embodiments, the administered shRNA includes a nucleic acid sequence that hybridizes under high stringency conditions to a Ccdc80 gene sequence of SEQ ID NO: 3. The administered anti-obesity shRNA may include the nucleic acid sequence of SEQ ID NO: 7. In some other embodiments, the administered anti-obesity shRNA is at least 85%, 90%, 95% or more identical to SEQ ID NO: 7.

In some embodiments, an anti-obesity polynucleotide that prevents or reduces at least one of Ccdc80 gene transcription or translation of Ccdc80 mRNA is DNA. In some further embodiments, the anti-obesity polynucleotide is linked to a peptide or antibody that binds to at least one cell surface receptor or antigen of the cell.

In some other embodiments, an anti-obesity agent that prevents or decreases Ccdc80 gene expression or Ccdc80 protein expression or activity is an agent that prevents or reduces the activity of Ccdc80 protein. An example of such an anti-obesity agent is an antibody against Ccdc80 protein.

The present invention also provides a method of screening for an agent that modulates adipogenesis. This method includes providing a cell that expresses the Ccdc80 gene; contacting the cell with a candidate agent; and evaluating the ability of the candidate agent to modulate the expression or activity of the Ccdc80 gene or Ccdc80 protein in the cell. A candidate agent that modulates this expression or activity is an agent that modulates adipogenesis. In some embodiments, the candidate agent is excluded for its ability to directly modulate the expression or activity of the Ccdc80 gene or Ccdc80 protein.

Another aspect of the present invention relates to the use of an agent that modulates the expression or activity of the Ccdc80 gene or Ccdc80 protein in the manufacture of a medicament for the treatment of a condition selected from obesity, insulin resistance, or type 2 diabetes. The agent may, for example, be a compound, a protein, a peptide, an antibody, an aptamer, or a polynucleotide, as described above.

A further aspect of the present invention relates to a pharmaceutical composition comprising an agent that modulates the expression or activity of the Ccdc80 gene or Ccdc80 protein; and a pharmaceutically acceptable carrier. An agent that modulates the expression or activity of the Ccdc80 gene or Ccdc80 protein may be alternatively referred to herein as a Ccdc80 modulator. In some embodiments, the agent in the pharmaceutical composition may be a compound, a protein, a peptide, an antibody, an aptamer, or a polynucleotide, as described above. In particular embodiments, the agent in the pharmaceutical composition is an shRNA, such as the one comprising the nucleic acid sequence of SEQ ID NO: 7. In some embodiments, the agent in the pharmaceutical composition is at least 85%, 90%, 95% or more identical to SEQ ID NO: 7. As described herein, alternatively, a vector, such as a retroviral vector used to express the shRNA, may be employed in the compositions and methods of the present invention.

Another aspect is for a method for the treatment of a mammal suffering from a condition selected from obesity, insulin resistance, or type 2 diabetes comprising administering to the mammal in need thereof a therapeutically effective amount of a Ccdc80 modulator.

A further aspect is for a method of identifying a Ccdc80 receptor comprising: a) providing Ccdc80 polypeptide to an adipocytic cell suspected of containing a Ccdc80 receptor; b) identifying specific binding of the Ccdc80 polypeptide to the adipocytic cell; and c) isolating the source of the specific binding.

A still further aspect is for a method of reducing proliferation of adipocytic cells comprising contacting the adipocytic cells with an effective amount of a Ccdc80 modulator.

An additional aspect is for a method of reducing lipid accumulation comprising contacting an adipocytic cell with an effective amount of a Ccdc80 modulator.

Another aspect is for a method of reducing adipogenesis of adipocytic cells comprising contacting the adipocytic cells with an effective amount of a Ccdc80 modulator.

A further aspect is for a method of regulating glucose homeostasis and/or lipid homeostasis in a mammal comprising administering to the mammal in need thereof a therapeutically effective amount of a Ccdc80 antibody, Ccdc80 antisense molecule, or a Ccdc80 antagonist.

Another aspect is for a method of screening for Ccdc80 mimics comprising: a) providing a candidate mimic and a Ccdc80 polypeptide; and b) determining whether the candidate mimic competes with Ccdc80 polypeptide in an assay designed to assess Ccdc80 polypeptide activity in an adipocytic cell.

An additional aspect is for a method of screening for modulators that affect Ccdc80 activity comprising: a) providing a candidate modulator and a Ccdc80 polypeptide; and b) determining whether the candidate modulator interferes with or enhances Ccdc80 adipocytic activity.

Other objects and advantages of the present invention will become apparent to those skilled in the art upon reference to the detailed description that hereinafter follows.

### BRIEF DESCRIPTION OF THE FIGURES

FIG. 1A is a bar graph showing the tissue distribution of Ccdc80 mRNA in normal mouse tissues: brown adipose tissue (BAT), brain, colon, white adipose tissue (WAT), skeletal muscle (SkM), heart, kidney, liver, small intestine (SI), spleen and stomach.

FIG. 1B is a bar graph showing the expression of mouse Ccdc80 mRNA in proliferating 3T3-L1 fibroblasts (preadipocyte) and fully differentiated adipocytes.

FIG. 1C is a bar graph showing expression of mouse Ccdc80 mRNA in white adipose tissue of fed and fasted (24 hr) mice. The graph shows significant down regulation of Ccdc80 mRNA in the mice that had been fasted for 24 hrs.

FIG. 1D is a bar graph showing expression of mouse Ccdc80 mRNA in white adipose tissue of wild-type and ob/ob mice. The graph shows significant down regulation of Ccdc80 mRNA in white adipose tissue of ob/ob mice as compared to wild-type mice.

FIG. 1E is a bar graph showing expression of mouse Ccdc80 mRNA in white adipose tissue of ob/ob mice treated with vehicle or thiazolidinedione (TZD). The graph shows significant up regulation of Ccdc80 mRNA shown in FIG. 1D upon treatment with TZD.

FIG. 1F is a bar graph showing expression of mouse CCDC80 mRNA in primary adipocytes or the stromal-vascular fraction isolated from epididymal white adipose tissue of C57BI/6J mice.

FIG. 1G is a bar graph showing tissue distribution of human Ccdc80 mRNA.

FIG. 2A is an SDS-polyacrylamide gel showing secretion of full-length Ccdc80 (˜140-kDa; denoted by an arrow) from 293T cells transfected with a plasmid encoding Ccdc80-tagged with the FLAG epitope (Ccdc80-FLAG). Conditioned medium was analyzed by silver staining. Identity of Ccdc80 was confirmed by mass spectrometry analysis.

FIG. 2B is a Western blot showing secretion of full-length (˜140-kDa, upper arrow) and cleaved fragments (˜95-kDa and ˜50-kDa, middle and lower arrow, respectively) of Ccdc80. Conditioned medium from 293T cells expressing a FLAG-tagged version of Ccdc80 (Ccdc80-FLAG) before (Pre-IP) and after (Post-IP) immunoprecipitation with an anti-FLAG M2 resin was analyzed by western blotting using an anti-FLAG antibody.

FIG. 2C is a Western blot showing that cleavage of Ccdc80 is partially prevented by the addition of protease inhibitors. 293T cells expressing Ccdc80-FLAG were incubated in the presence of a cocktail of protease inhibitors for 48 hrs. Conditioned medium from the cells was analyzed by western blotting using an anti-FLAG antibody.

FIG. 2D is a Western blot showing secretion of Ccdc80 by 3T3-L1 adipocytes. Conditioned medium from 293T cells expressing Ccdc80-FLAG or 3T3-L1 preadipocytes and adipocytes were analyzed by Western blotting using an antibody that recognizes Ccdc80. 3T3-L1 adipocytes secrete full-length (˜140-kDa) and a cleaved fragment (˜50-kDa) of Ccdc80 (indicated by arrows).

FIG. 3A is a schematic representation of the 3T3-L1 adipocyte differentiation protocol. Gene expression was analyzed during specific phases of differentiation (i.e. proliferation, growth arrest, clonal expansion and terminal differentiation) as indicated by arrows.

FIG. 3B is a bar graph showing Ccdc80 mRNA expression in 3T3-L1 cells during proliferation, growth arrest, clonal expansion and terminal differentiation. The graph shows that Ccdc80 is expressed in a biphasic manner in 3t3-L1 cells during differentiation.

FIG. 3C is a bar graph showing Ccdc80 mRNA expression in growth-arrested cells (Time=0 hr) and upon induction of differentiation by the addition of adipogenic inducers (dexamethasone, IBMX and insulin) for 8, 16 and 24 hr. The graph shows Ccdc80 repression during clonal expansion.

FIG. 3D is a bar graph showing the effect of adipogenic inducers on Ccdc80 expression. Growth-arrested 3T3-L1 cells were left untreated or treated with one or more adipogenic inducers for 96 hr. Ccdc80 mRNA expression (panels B-D) was measured by real-time PCR. (n=3 per group).

FIG. 4A is a bar graph showing the effect of silencing of Ccdc80 by RNA interference on Ccdc80 mRNA expression. Stable 3T3-L1 cell lines transduced with retrovirus encoding a non-silencing shRNA (white bars) or an shRNA against mouse Ccdc80 (black bars) were created. Ccdc80 expression was determined by real-time PCR during proliferation, growth arrest, clonal expansion and terminal differentiation. *p<0.05 vs Non-silencing shRNA. The graph shows that silencing of Ccdc80 by RNA interference markedly decreased Ccdc80 mRNA levels

FIG. 4B is a Western blot showing the effect of silencing of Ccdc80 by RNA interference on secretion of Ccdc80. Conditioned medium from growth-arrested and terminally differentiated 3T3-L1 was analyzed by western blotting using an antibody that recognizes Ccdc80. The full-length (˜140-kDa) and a cleaved fragment (˜50-kDa) of Ccdc80 in conditioned medium from terminally differentiated adipocytes are indicated by arrows. The graph shows that silencing of Ccdc80 by RNA interference markedly blunted the secretion of the protein.

FIG. 4C are bar graphs of the mRNA expression profile of genes involved in adipogenesis, metabolism and signaling. Samples were analyzed at the end of the differentiation protocol using a mouse genome microarray. *p<0.05 vs Non-silencing shRNA.

FIG. 4D are bar graphs of normalized mRNA expression levels of adipogenic markers during differentiation. Expression of aP2, C/EBPα and PPARγ was determined by real-time PCR during proliferation, growth arrest, clonal expansion and terminal differentiation. *p<0.05 vs Non-silencing shRNA.

FIG. 4E is a Western blot showing the activation of Akt and ERK by insulin. Serum-deprived 3T3-L1 cells were left untreated or treated with insulin (10 nM) for 10 min. Cell lysates were analyzed by western blotting. Phosphorylation of Akt at Ser473 and ERK½ at Thr202/Tyr204 was determined using phospho-specific antibodies. Total levels of Akt and ERK½ are also shown.

FIG. 5A is a bar graph showing Ccdc80 mRNA expression as determined by real-time PCR in 3T3-L1 cells infected with adenovirus at a MOI of 500, 1000 or 2000. *p<0.05 vs Ad-LacZ. 3T3-L1 cells were infected with adenovirus encoding either LacZ (Ad-LacZ, white bars) or mouse Ccdc80 (Ad-Ccdc80, black bar) at the various multiplicity of infection (MOI).

FIG. 5B is a Western blot showing secretion of Ccdc80. Conditioned medium from growth-arrested and terminally differentiated 3T3-L1 infected with adenovirus at a MOI of 2000 was analyzed by western blotting using an antibody that recognizes Ccdc80. The full-length (˜140-kDa) and cleaved fragments (˜50-kDa and ˜25-kDa) of Ccdc80 in conditioned medium from growth-arrested and terminally differentiated adipocytes are indicated by arrows.

FIG. 5C are bar graphs showing normalized mRNA Expression of adipogenic markers. Expression of aP2, C/EBPα and PPARγ was determined by real-time PCR in 3T3-L1 cells infected with adenovirus at MOI of 1000 or 2000. *p<0.05 vs Ad-LacZ.

FIG. 5D are bar graphs showing induction of adipogenic markers during differentiation. Expression of aP2, C/EBPα and PPARγ was determined by real-time PCR in 3T3-L1 cells infected with adenovirus at a MOI of 2000 during proliferation, growth arrest, clonal expansion and terminal differentiation. *p<0.05 vs Ad-LacZ.

FIG. 6A are bar graphs showing the normalized mRNA expression levels of Wnt/β-catenin pathway components. Stable 3T3-L1 cell lines transduced with retrovirus encoding a non-silencing shRNA (white bars) or an shRNA against mouse Ccdc80 (black bars) were created and employed in these experiments. Gene expression was determined by real-time PCR using a low-density array. *p<0.05 vs Non-silencing shRNA.

FIG. 6B are bar graphs showing the normalized mRNA expression levels of TCF/LEF transcription factors. Stable 3T3-L1 cell lines transduced with retrovirus encoding a non-silencing shRNA (white bars) or an shRNA against mouse Ccdc80 (black bars) were created and employed in these experiments. Gene expression was determined by real-time PCR using a low-density array. *p<0.05 vs Non-silencing shRNA.

FIG. 6C are bar graphs showing Wnt/β-catenin targets. Stable 3T3-L1 cell lines transduced with retrovirus encoding a non-silencing shRNA (white bars) or an shRNA against mouse Ccdc80 (black bars) were created and employed in these experiments. Gene expression was determined by real-time PCR using a low-density array. *p<0.05 vs Non-silencing shRNA.

FIG. 7A is a bar graph showing Cyclin D1 repression during clonal expansion. Cyclin D1 expression was determined by real-time PCR in 3T3-L1 stably transduced with retrovirus encoding a non-silencing shRNA (white bars) or an shRNA against mouse Ccdc80 (black bars) [Knockdown; left portion of the graph] or in 3T3-L1 infected with adenovirus encoding either LacZ (Ad-LacZ, white bars) or mouse Ccdc80 (Ad-Ccdc80, black bar) at a MOI of 2000 [Overexpression; right portion of the graph]. Data are presented as % change in Cyclin D1 expression from growth arrest to clonal expansion. *p<0.05 vs Non-silencing shRNA.

FIG. 7B is a bar graph showing TOPFLASH reporter activity during clonal expansion. 3T3-L1 stably transduced with retrovirus encoding a non-silencing shRNA (white bars) or an shRNA against mouse Ccdc80 (black bars) were transfected with a TOPFLASH reporter plasmid. Luciferase activity was measured in growth-arrested cells (Time=0 hr) and upon induction of differentiation by the addition of adipogenic inducers (dexamethasone, IBMX and insulin) for 24, 48 and 96 hr.

FIG. 7C is a bar graph showing TOPFLASH reporter activity. β-catenin protein expression is shown above the graph. HepG2 cells were infected with adenovirus encoding either GFP (Ad-GFP, white bars) or human Ccdc80 (Ad-Ccdc80, black bar) at MOI of 100, 250 and 500 were transfected with a TOPFLASH reporter plasmid. β-catenin protein expression and luciferase activity were measured 24 hr later.

FIG. 7D is a schematic representation of a proposed mechanism by which Ccdc80 regulates adipogenesis. Preadipocytes express high levels of Ccdc80 upon reaching growth arrest, which are required for the efficient repression of Wnt/β-catening signaling during clonal expansion and the subsequent induction/activation of C/EBPα and PPARγ and lipid accumulation during terminal differentiation.

### BRIEF DESCRIPTION OF SEQUENCES

SEQ ID NO:1 is a forward Ccdc80 primer.

SEQ ID NO:2 is a reverse Ccdc80 primer.

SEQ ID NO:3 encodes a short hairpin RNA (shRNA) against mouse Ccdc80.

SEQ ID NO:4 encodes a non-silencing shRNA, which does not match any known mammalian genes as determined via nucleotide alignment/BLAST of target 22-mer sequence.

SEQ ID NO:5 is a Ccdc80 peptide.

SEQ ID NO:6 is a Ccdc80 peptide.

SEQ ID NO:7 is a short hairpin RNA (shRNA) against mouse Ccdc80.

SEQ ID NO:7 is encoded by SEQ ID NO:3 above.

### DETAILED DESCRIPTION OF THE INVENTION

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, e.g., Molecular Cloning: A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); U.S. Pat. No. 4,683,195; Nucleic Acid Hybridization (B. D. Hames & S. J. Higgins eds. 1984); Transcription and Translation (B. D. Hames & S. J. Higgins eds. 1984); Culture of Animal Cells (R. I. Freshney, Alan R. Liss, Inc., 1987); Immobilized Cells and Enzymes (IRL Press, 1986); B. Perbal, A Practical Guide to Molecular Cloning (1984); Methods in Enzymology (Academic Press, Inc., N.Y.); Gene Transfer Vectors for Mammalian Cells (J. H. Miller and M. P. Calos eds., 1987, Cold Spring Harbor Laboratory); Methods in Enzymology, Vols. 154 and 155 (Wu et al. eds.), Immunochemical Methods in Cell and Molecular Biology (Mayer and Walker, eds., Academic Press, London, 1987); Handbook of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Ccdc80 is expressed and regulated in a manner consistent with an adipokine. Both mouse and human Ccdc80 are expressed preferentially in white adipose tissue. Mouse Ccdc80 mRNA is expressed at higher levels in adipocytes compared to stromal cells and is up-regulated during adipocyte differentiation. Expression of Ccdc80 in white adipose tissue is significantly decreased upon fasting and is also decreased in ob/ob mice, a genetic model of obesity and type 2 diabetes. Treatment of ob/ob mice with the insulin sensitizing agent rosiglitazone improves both their diabetes and also upregulates Ccdc80. This pattern of expression and regulation suggests a role for Ccdc80 in metabolic disorders. Contrary to what has been reported in the literature, human Ccdc80 can be secreted efficiently into the medium, consistent with Ccdc80 acting as an adipokine in humans.

Reduction of Ccdc80 expression by stable retroviral expression of an shRNA against mouse Ccdc80 in 3T3-L1 cells increased the proliferation of pre-adipocyte and reduced their conversion into mature adipocytes.

Furthermore, exaggerated overexpression of Ccdc80 can inhibit adipogenesis.

### I. DEFINITIONS

In the context of this disclosure, a number of terms shall be utilized.

As used herein, the term “about” or “approximately” means within 20%, preferably within 10%, and more preferably within 5% of a given value or range.

“Adipocytic cells” include preadipocytes, adipocytes, mesenchymal stem cells, embryonic stem cells, and embryonic fibroblasts.

The term “adipogenesis” as used herein refers to the production of fat, the deposition of fat, the generation of new fat cells through adipocyte differentiation or to the conversion of carbohydrate or protein to fat.

The term “adipokine” as used herein refers to a protein secreted from adipose tissues with autocrine, paracrine, and/or endocrine functions.

An “antibody” includes an immunoglobulin molecule capable of binding an epitope present on an antigen. As used herein, the term encompasses not only intact immunoglobulin molecules such as monoclonal and polyclonal antibodies, but also anti-idotypic antibodies, mutants, fragments, fusion proteins, bi-specific antibodies, humanized proteins, and modifications of the immunoglobulin molecule that comprise an antigen recognition site of the required specificity.

The term “Ccdc80” or “coiled-coil domain containing 80” is used herein interchangeably with its aliases URB, DRO1, SSG1, and EQUARIN. Exemplary GenBank® accession numbers for Ccdc80 sequences include the following: human (Homo sapiens, NM_{—}199511), mouse (Mus musculus, NM_{—}026439), rat (Rattus norvegicus, NM_{—}022543), chicken (Gallus gallus, NM_{—}204431).

The term “cDNA” includes complementary DNA that is mRNA molecules present in a cell or organism made into cDNA with an enzyme such as reverse transcriptase. A “cDNA library” includes a collection of mRNA molecules present in a cell or organism, converted into cDNA molecules with the enzyme reverse transcriptase, then inserted into vectors. The library can then be probed for the specific cDNA (and thus mRNA) of interest.

As used herein, a Ccdc80 “chimeric protein” or “fusion protein” comprises a Ccdc80 polypeptide operably linked to a non-Ccdc80 polypeptide. A “Ccdc80 polypeptide” refers to a polypeptide having an amino acid sequence corresponding to Ccdc80 polypeptide, whereas a “non-Ccdc80 polypeptide” refers to a polypeptide having an amino acid sequence corresponding to a protein which is not substantially homologous to the Ccdc80 protein, for example, a protein which is different from the Ccdc80 protein and which is derived from the same or a different organism. Within a Ccdc80 fusion protein, the Ccdc80 polypeptide can correspond to all or a portion of a Ccdc80 protein. In a preferred embodiment, a Ccdc80 fusion protein comprises at least one biologically active portion of a Ccdc80 protein. Within the fusion protein, the term “operably linked” is intended to indicate that the Ccdc80 polypeptide and the non-Ccdc80 polypeptide are fused in-frame to each other. The non-Ccdc80 polypeptide can be fused to the N-terminus or C-terminus of the Ccdc80 polypeptide.

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a Ccdc80 polypeptide is preferably replaced with another amino acid residue from the same side chain family.

The terms “effective amount”, “therapeutically effective amount”, and “effective dosage” as used herein refer to the amount of a molecule that, when administered to a mammal in need, is effective to at least partially ameliorate conditions related to, for example, obesity, insulin resistance, and/or type 2 diabetes, and/or is effective to at least partially modulate, for example, glucose levels and/or lipid homeostatis.

As used herein, the term “expression” includes the process by which a gene is transcribed into mRNA. As used herein, the term “expression” also includes the process by which an mRNA is translated into an amino acid sequence. As used herein, the term “expression” further includes the process by which polynucleotides are transcribed into mRNA and translated into peptides, polypeptides, or proteins. As used herein, the phrase “modulates the expression or activity of the Ccdc80 gene or Ccdc80 protein” is intended to include an increase or decrease in mRNA or polypeptide levels, as well as an increase or decrease in protein activity. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook, J., Fritsh, E. F., and Maniatis, T., Molecular Cloning: A Laboratory Manual, 2nd ed., Cold Spring Harbor Laboratory, Cold Spring Harbor Laboratory Press, Cold Spring Harbor, N.Y., 1989). Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

The term “expression construct” means any double-stranded DNA or double-stranded RNA designed to transcribe an RNA, e.g., a construct that contains at lease one promoter operably linked to a downstream gene or coding region of interest (e.g., a cDNA or genomic DNA fragment that encodes a protein, or any RNA of interest). Transfection or transformation of the expression construct into a recipient cell allows the cell to express RNA or protein encoded by the expression construct. An expression construct may be a genetically engineered plasmid, virus, or an artificial chromosome derived from, for example, a bacteriophage, adenovirus, retrovirus, poxvirus, or herpesvirus. An expression construct can be replicated in a living cell, or it can be made synthetically. For purposes of this application, the terms “expression construct”, “expression vector”, “vector”, and “plasmid” are used interchangeably to demonstrate the application of the invention in a general, illustrative sense, and are not intended to limit the invention to a particular type of expression construct. Further, the term expression construct or vector is intended to also include instances wherein the cell utilized for the assay already endogenously comprises such DNA sequence.

A “gene” includes a polynucleotide containing at least one open reading frame that is capable of encoding a particular polypeptide or protein after being transcribed and translated. Any of the polynucleotide sequences described herein may be used to identify larger fragments or full-length coding sequences of the gene with which they are associated. Methods of isolating larger fragment sequences are known to those of skill in the art, some of which are described herein.

The term “genetically modified” includes a cell containing and/or expressing a foreign gene or nucleic acid sequence which in turn modifies the genotype or phenotype of the cell or its progeny. This term includes any addition, deletion, or disruption to a cell's endogenous nucleotides.

The term “gene product” as used herein, unless otherwise indicated, refers to a product produced by a gene when that gene is transcribed or translated. A “gene product” may be any transcription or translational product derived from a specific gene locus. Typically, the term refers to a nucleic acid, such as, for example, a messenger RNA, or a protein or a polypeptide. A “gene product” includes an amino acid sequence (e.g., peptide or polypeptide) generated when a gene is transcribed and translated.

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. A heterologous expression regulatory element is such an element operably associated with a different gene than the one it is operably associated with in nature.

The term “homologous” as used herein refers to the sequence similarity between two polymeric molecules, e.g., between two nucleic acid molecules, e.g., two DNA molecules or two RNA molecules, or between two polypeptide molecules. When a nucleotide or amino acid position in both of the two molecules is occupied by the same monomeric nucleotide or amino acid, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′ATTGCC3′ and 5′TATGCG3′ share 50% homology. By the term “substantially homologous” as used herein, is meant DNA or RNA which is about 50% homologous, in another embodiment about 60% homologous, in another embodiment about 70% homologous, in another embodiment about 80% homologous, in another embodiment about 85% homologous, in another embodiment about 90% homologous, in another embodiment about 95% homologous to the desired nucleic acid.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The residues at corresponding positions are then compared and when a position in one sequence is occupied by the same residue as the corresponding position in the other sequence, then the molecules are identical at that position. The percent identity between two sequences, therefore, is a function of the number of identical positions shared by two sequences (i.e., % identity=# of identical positions/total # of positions×100). The percent identity between the two sequences is a function of the number of identical positions shared by the sequences, taking into account the number of gaps, and the length of each gap, which are introduced for optimal alignment of the two sequences.

The comparison of sequences and determination of percent identity between two sequences can be accomplished using a mathematical algorithm. A non-limiting example of a mathematical algorithm utilized for comparison of sequences is the algorithm of Karlin S and Altschul S F, Proc. Natl. Acad. Sci. USA 87:2264-68 (1990), modified as in Karlin S and Altschul S F, Proc. Natl. Acad. Sci. USA 90:5873-77 (1993). Such an algorithm is incorporated into the NBLAST and XBLAST programs (version 2.0) of Altschul S F et al., J. Mol. Biol. 215:403-10 (1990). BLAST nucleotide searches can be performed with the NBLAST program score=100, wordlength=12 to obtain homologous nucleotide sequences. BLAST protein searches can be performed with the XBLAST program, score=50, wordlength=3 to obtain amino acid sequences homologous to the protein molecules of the invention. To obtain gapped alignments for comparison purposes, Gapped BLAST can be utilized as described in Altschul S F et al., Nucleic Acids Res. 25:3389-3402 (1997). When utilizing BLAST and Gapped BLAST programs, the default parameters of the respective programs (e.g., XBLAST and NBLAST) can be used. Another preferred, non-limiting algorithm utilized for the comparison of sequences is the algorithm of Myers E W and Miller W, Comput. Appl. Biosci. 4:11-17 (1988). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Another non-limiting example of a mathematical algorithm utilized for the alignment of protein sequences is the Lipman-Pearson algorithm (Lipman D J and Pearson W R, Science 227:1435-41 (1985)). When using the Lipman-Pearson algorithm, a PAM250 weight residue table, a gap length penalty of 12, a gap penalty of 4, and a Kutple of 2 can be used. A preferred, non-limiting example of a mathematical algorithm utilized for the alignment of nucleic acid sequences is the Wilbur-Lipman algorithm (Wilbur W J and Lipman D J, Proc. Natl. Acad. Sci. USA 80:726-30 (1983)). When using the Wilbur-Lipman algorithm, a window of 20, gap penalty of 3, Ktuple of 3 can be used. Both the Lipman-Pearson algorithm and the Wilbur-Lipman algorithm are incorporated, for example, into the MEGALIGN program (e.g., version 3.1.7) which is part of the DNASTAR sequence analysis software package.

Additional algorithms for sequence analysis are known in the art, and include ADVANCE and ADAM, described in Torelli A and Robotti C A, Comput. Appl. Biosci. 10:3-5 (1994); and FASTA, described in Pearson W R and Lipman D J, Proc. Natl. Acad. Sci. USA 85:2444-48 (1988).

In one embodiment, the percent identity between two amino acid sequences is determined using the GAP program in the GCG software package, using either a Blosum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package, using a NWSgapdna. CMP matrix and a gap weight of 40, 50, 60, 70, or 80 and a length weight of 1, 2, 3, 4, 5, or 6.

Protein alignments can also be made using the Geneworks global protein alignment program (e.g., version 2.5.1) with the cost to open gap set at 5, the cost to lengthen gap set at 5, the minimum diagonal length set at 4, the maximum diagonal offset set at 130, the consensus cutoff set at 50% and utilizing the Pam 250 matrix.

A “host cell” is intended to include any individual cell or cell culture which can be or has been a recipient for vectors or for the incorporation of exogenous nucleic acid molecules, polynucleotides, and/or proteins. It also is intended to include progeny of a single cell. The progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, insect cells, animal cells, and mammalian cells, e.g., murine, rat, simian, or human cells.

“Hybridization” includes a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. The stringency of a hybridization reaction includes the difficulty with which any two nucleic acid molecules will hybridize to one another. Under stringent conditions, nucleic acid molecules at least 65%, 70%, 75%, 80%, 85%, 90%, 95% or more identical to each other remain hybridized to each other, whereas molecules with low percent identity cannot remain hybridized. A preferred, non-limiting example of highly stringent hybridization conditions are hybridization in 6× sodium chloride/sodium citrate (SSC) at about 45° C., followed by one or more washes in 0.2×SSC, 0.1% SDS at 50° C., preferably at 55° C., more preferably at 60° C., and even more preferably at 65° C.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide if hybridization can occur between one of the strands of the first polynucleotide and the second. “Complementarity” or homology is quantifiable in terms of the proportion of bases in opposing strands that are expected to hydrogen bond with each other, according to generally accepted base-pairing rules.

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes, for example, a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “mammal” refers to a human, a non-human primate, canine, feline, bovine, ovine, porcine, murine, or other veterinary or laboratory mammal. Those skilled in the art recognize that a therapy which reduces the severity of a pathology in one species of mammal is predictive of the effect of the therapy on another species of mammal.

The term “modulates” as in “an agent that modulates the expression or activity of the Ccdc80 gene or Ccdc80 protein” means that the agent directly or indirectly modulates this expression or activity. As used herein, the term “directly modulates” as in “an agent that directly modulates the expression or activity of the Ccdc80 gene or Ccdc80 protein” means that the agent or a derivative thereof directly binds or directly interacts with a Ccdc80 protein or a Ccdc80 polynucleotide (e.g., gene or mRNA encoded by a gene), thereby inhibiting or stimulating the functional activity of Ccdc80 protein. For example, and without being bound to any one theory, the functional activity of Ccdc80 protein may be sequestered or inhibited by an agent that directly interacts with Ccdc80 protein, such as a neutralizing Ccdc80 antibody, or a small molecule. As another example, translation of Ccdc80 mRNA may be prevented or reduced by an agent, such as a Ccdc80-specific RNAi, e.g., a small interfering RNA (siRNA) or a short hairpin RNA (shRNA), that specifically silences the expression of the Ccdc80 gene. In some embodiments, the agent “directly modulates” by binding to the Ccdc80 protein, Ccdc80 RNA or promoter of the Ccdc80 gene.

For example, rosiglitazone modulates Ccdc80, as shown in Example 2. However, since rosiglitazone is an anti-diabetic drug in the thiazolidinedione class of drugs and, like other thiazolidinediones, binds the intracellular receptor class of the peroxisome proliferator-activated receptors (PPARs), specifically PPARγ (i.e., rosiglitazone is a selective ligand of PPARγ and has no PPARα-binding action), it does not directly modulate Ccdc80.

The term “modulate” encompasses either a decrease or an increase in activity depending on the target molecule. For example, a Ccdc80 modulator is considered to modulate the activity of Ccdc80 if the presence of such Ccdc80 modulator results in an increase or decrease in Ccdc80 activity. As used herein, the phrase “modulates the expression or activity of the Ccdc80 gene or Ccdc80 protein” is intended to include an increase or decrease in mRNA or polypeptide levels, as well as an increase or decrease in protein activity. Such an increase or decrease can be of varying magnitude, provided that it is statistically significant. For example, a statistically significant change, such as a decrease or increase in the level of Ccdc80 protein activity in the presence of a compound (relative to what is detected in the absence of the compound) is indicative of the compound being a Ccdc80 modulator. The increase or decrease can be of various scales as compared to what is observed in a control assay. A decrease in mRNA or polypeptide levels, or a decrease in protein activity may be complete or partial. A decrease may be complete or partial when compared to a reference level in a given cell or cell type.

As used herein, a “naturally-occurring” nucleic acid molecule refers to an RNA or DNA molecule having a nucleotide sequence that occurs in nature (e.g., encodes a natural protein).

The term “operably linked” means that a nucleic acid molecule, e.g., DNA, and one or more regulatory sequences (e.g., a promoter or portion thereof) are connected in such a way as to permit transcription of mRNA from the nucleic acid molecule or permit expression of the product (i.e., a polypeptide) of the nucleic acid molecule when the appropriate molecules are bound to the regulatory sequences. Within a fusion construct, the term “operably linked” is intended to indicate that the Ccdc80 polynucleotide and a non-Ccdc80 polynucleotide are fused in-frame to each other. The non-Ccdc80 polynucleotide can be fused 3′ or 5′ to the Ccdc80 polynucleotide.

As used herein, the terms “polynucleotide” and “oligonucleotide” are used interchangeably, and include polymeric forms of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, double stranded RNA (dsRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form.

As used herein, the term “shRNA” refers to short hairpin RNA. A short hairpin RNA is a sequence of RNA that makes a tight hairpin turn that can be used to silence gene expression via RNA interference. As used herein, the term “shRNA”, as in a composition comprising shRNA, or a method of use of shRNA, is intended to include use in the composition or method of an shRNA, as well as vectors (e.g., viral vectors) expressing shRNA, to inhibit gene expression.

The term “siRNA”, as used herein, refers to small interfering RNA, sometimes known as short interfering RNA or silencing RNA. In general, these terms refer to a class of RNA molecules that interfere with the expression of specific genes.

A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule.

The term “polypeptide” includes a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” includes either natural and/or unnatural or synthetic amino acids, including glycine and both the D or L optical isomers, and amino acid analogs and peptidomimetics. A peptide of three or more amino acids is commonly referred to as an oligopeptide. Peptide chains of greater than three or more amino acids are referred to as a polypeptide or a protein.

A “primer” includes a short polynucleotide, generally with a free 3′-OH group that binds to a target or “template” present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or “set of primers” consisting of an “upstream” and a “downstream” primer, and a catalyst of polymerization, such as a DNA polymerase, and typically a thermally-stable polymerase enzyme. Methods for PCR are well known in the art, and are taught, for example, in MacPherson M et al., PCR: A Practical Approach, IRL Press at Oxford University Press (1991). All processes of producing replicate copies of a polynucleotide, such as PCR or gene cloning, are collectively referred to herein as “replication”. A primer can also be used as a probe in hybridization reactions, such as Southern or Northern blot analyses (see, e.g., Sambrook, J., Fritsh, E. F., and Maniatis, T. Molecular Cloning: A Laboratory Manual, 2nd ed., Cold Spring Harbor Laboratory, Cold Spring Harbor Laboratory Press, Cold Spring Harbor, N.Y., 1989).

A “probe” when used in the context of polynucleotide manipulation includes an oligonucleotide that is provided as a reagent to detect a target present in a sample of interest by hybridizing with the target. Usually, a probe will comprise a label or a means by which a label can be attached, either before or subsequent to the hybridization reaction. Suitable labels include, but are not limited to, radioisotopes, fluorochromes, chemiluminescent compounds, dyes, and proteins, including enzymes.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined, for example, by mapping with nuclease S1), as well as protein binding domains (consensus sequences) responsible for the binding of RNA polymerase.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure; and more preferably still at least 99% pure. Purity can be evaluated by chromatography, gel electrophoresis, immunoassay, composition analysis, biological assay, and other methods known in the art.

The term “test compound” includes compounds with known chemical structure but not necessarily with a known function or biological activity. Test compounds could also have unidentified structures or be mixtures of unknown compounds, for example from crude biological samples such as plant extracts. Large numbers of compounds could be randomly screened from “chemical libraries” which refers to collections of purified chemical compounds or collections of crude extracts from various sources. The chemical libraries may contain compounds that were chemically synthesized or purified from natural products. The compounds may comprise inorganic or organic small molecules or larger organic compounds such as, for example, proteins, peptides, glycoproteins, steroids, lipids, phospholipids, nucleic acids, and lipoproteins. The amount of compound tested can very depending on the chemical library, but, for purified (homogeneous) compound libraries, 10 μM is typically the highest initial dose tested. Methods of introducing test compounds to cells are well known in the art.

### II. ISOLATED POLYNUCLEOTIDES ENCODING Ccdc80 OR PORTIONS THEREOF

In practicing the methods of the invention, various agents can be used to modulate the activity and/or expression of Ccdc80 in a cell. In one embodiment, an agent is a nucleic acid molecule encoding a Ccdc80 polypeptide or a portion thereof, including, for example, human (Homo sapiens, NM_{—}199511), mouse (Mus musculus, NM_{—}026439), rat (Rattus norvegicus, NM_{—}022543), chicken (Gallus gallus, NM_{—}204431).

A polynucleotide can be amplified using cDNA, mRNA or alternatively, genomic DNA, as a template and appropriate oligonucleotide primers according to standard PCR amplification techniques. The polynucleotide so amplified can be cloned into an appropriate vector and characterized by DNA sequence analysis. Furthermore, oligonucleotides corresponding to Ccdc80 polynucleotide sequences can be prepared by standard synthetic techniques, e.g., using an automated DNA synthesizer.

Moreover, a Ccdc80 polynucleotide can comprise only a portion of a Ccdc80 full-length polynucleotide sequence, for example, a fragment which can be used as a probe or primer or a fragment encoding a biologically active portion of a Ccdc80 protein. The polynucleotide sequence determined from the cloning of Ccdc80 genes allows for the generation of probes and primers designed for use in identifying and/or cloning other Ccdc80 family members, as well as Ccdc80 family homologues from other species.

The probe/primer typically comprises a substantially purified oligonucleotide. In one embodiment, the oligonucleotide comprises a region of nucleotide sequence that hybridizes under stringent conditions to at least about 12 or 15, preferably about 20 or 25, more preferably about 30, 35, 40, 45, 50, 55, 60, 65, 75, 80, 85, 90, 95 or 100 consecutive polynucleotides of a sense sequence of a full-length Ccdc80 polynucleotide sequence or of a naturally occurring allelic variant or mutant of said full-length sequence. In another embodiment, a polynucleotide comprises a polynucleotide sequence which is at least about 100, 200, 300, 400, 500, 600, or 700 nucleotides in length and hybridizes under stringent hybridization conditions to a polynucleotide sequence of a full-length Ccdc80 polynucleotide sequence or a complement thereof.

A nucleic acid fragment encoding a “biologically active portion of a Ccdc80 protein” can be prepared by isolating a portion of a full-length Ccdc80 polynucleotide sequence which encodes a polypeptide having a Ccdc80 biological activity (e.g., modulating preadipocyte proliferation and/or modulating lipid accumulation), expressing the encoded portion of a Ccdc80 protein (e.g., by recombinant expression in vitro), and assessing the activity of the encoded portion of the Ccdc80 protein.

Another embodiment relates to antisense polynucleotides. Antisense polynucleotides are typically administered to a subject or generated in situ such that they hybridize with or bind to cellular mRNA and/or genomic DNA encoding a Ccdc80 protein to thereby inhibit expression of the protein, for example, by inhibiting transcription and/or translation. The hybridization can be by conventional nucleotide complementarity to form a stable duplex, or, for example, in the case of an antisense polynucleotide which binds to DNA duplexes, through specific interactions in the major groove of the double helix. An example of a route of administration of antisense polynucleotides of the invention include direct injection at a tissue site. Alternatively, antisense polynucleotides can be modified to target selected cells and then administered systemically. For example, for systemic administration, antisense molecules can be modified such that they specifically bind to receptors or antigens expressed on a selected cell surface, for example, by linking the antisense polynucleotides to peptides or antibodies which bind to cell surface receptors or antigens. The antisense polynucleotides can also be delivered to cells using the vectors described herein. To achieve sufficient intracellular concentrations of the antisense molecules, vector constructs in which the antisense polynucleotide is placed under the control of a strong pol II or pol III promoter are preferred.

In yet another embodiment, an antisense polynucleotide is an α-anomeric nucleic acid molecule. An α-anomeric nucleic acid molecule forms specific double-stranded hybrids with complementary RNA in which, contrary to the usual β-units, the strands run parallel to each other (Gaultier C et al., Nucleic Acids Res. 15:6625-41 (1987)). The antisense nucleic acid molecule can also comprise a 2′-o-methylribonucleotide (Inoue H et al., Nucleic Acids Res. 15:6131-48 (1987)) or a chimeric RNA-DNA analogue (Inoue H et al., FEBS Lett. 215:327-30 (1987)).

In still another embodiment, an antisense polynucleotide is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff J and Gerlach W L, Nature 334:585-91 (1988))) can be used to catalytically cleave Ccdc80 mRNA transcripts to thereby inhibit translation of Ccdc80 mRNA. A ribozyme having specificity for a Ccdc80-encoding nucleic acid can be designed based upon, for example, the nucleotide sequence of any of the Ccdc80 GenBank® sequences noted above. For example, a derivative of a Tetrahymena L-19 IVS RNA can be constructed in which the nucleotide sequence of the active site is complementary to the nucleotide sequence to be cleaved in a Ccdc80-encoding mRNA (see, e.g., U.S. Pat. Nos. 4,987,071 and 5,116,742). Alternatively, Ccdc80 mRNA can be used to select a catalytic RNA having a specific ribonuclease activity from a pool of RNA molecules (see, e.g., Bartel D and Szostak J W, Science 261:1411-18 (1993)).

Alternatively, gene expression can be inhibited by targeting nucleotide sequences complementary to the regulatory region of Ccdc80 (e.g., Ccdc80 promoter and/or enhancers) to form triple helical structures that prevent transcription of the Ccdc80 gene in target cells (see generally, Helene C, Anticancer Drug Des. 6:569-84 (1991); Helene C et al., Ann. N.Y. Acad. Sci. 660:27-36 (1992); Maher L J, Bioassays 14:807-15 (1992)).

In other embodiments, the oligonucleotide may include other appended groups such as peptides (e.g., for targeting host cell receptors in vivo), or agents facilitating transport across the cell membrane (see, e.g., Letsinger R L et al., Proc. Natl. Acad. Sci. USA 86:6553-56 (1989); Lemaitre M et al., Proc. Natl. Acad. Sci. USA 84:648-52 (1987); PCT Publication No. WO88/09810) or the blood-brain barrier (see, e.g., PCT Publication No. WO89/10134). In addition, oligonucleotides can be modified with hybridization-triggered cleavage agents (see, e.g., van der Krol A R et al., Biotechniques 6:958-76 (1988)) or intercalating agents (see, e.g., Zon G, Pharm. Res. 5:539-49 (1988)). To this end, the oligonucleotide may be conjugated to another molecule, (e.g., a peptide, hybridization triggered cross-linking agent, transport agent, or hybridization-triggered cleavage agent).

In one embodiment, Ccdc80 expression can be inhibited by short interfering RNAs (siRNA). The siRNA can be dsRNA having 19-25 nucleotides. siRNAs can be produced endogenously by degradation of longer dsRNA molecules by an RNase III-related nuclease called Dicer. siRNAs can also be introduced into a cell exogenously, or by transcription of an expression construct. Once formed, the siRNAs assemble with protein components into endoribonuclease-containing complexes known as RNA-induced silencing complexes (RISCs). An ATP-generated unwinding of the siRNA activates the RISCs, which in turn target the complementary mRNA transcript by Watson-Crick base-pairing, thereby cleaving and destroying the mRNA. Cleavage of the mRNA takes place near the middle of the region bound by the siRNA strand. This sequence specific mRNA degradation results in gene silencing.

At least two ways can be employed to achieve siRNA-mediated gene silencing. First, siRNAs can be synthesized in vitro and introduced into cells to transiently suppress gene expression. Synthetic siRNA provides an easy and efficient way to achieve RNAi. siRNAs are duplexes of short mixed oligonucleotides which can include, for example, 19 RNAs nucleotides with symmetric dinucleotide 3′ overhangs. Using synthetic 21 bp siRNA duplexes (e.g., 19 RNA bases followed by a UU or dTdT 3′ overhang), sequence specific gene silencing can be achieved in mammalian cells. These siRNAs can specifically suppress targeted gene translation in mammalian cells without activation of DNA-dependent protein kinase (PKR) by longer double-stranded RNAs (dsRNA), which may result in non-specific repression of translation of many proteins.

Second, siRNAs can be expressed in vivo from vectors. This approach can be used to stably express siRNAs in cells or transgenic animals. In one embodiment, siRNA expression vectors are engineered to drive siRNA transcription from polymerase III (pol III) transcription units. Pol III transcription units are suitable for hairpin siRNA expression because they deploy a short AT rich transcription termination site that leads to the addition of 2 bp overhangs (e.g., UU) to hairpin siRNAs—a feature that is helpful for siRNA function. The Pol III expression vectors can also be used to create transgenic mice that express siRNA.

In another embodiment, siRNAs can be expressed in a tissue-specific manner. Under this approach, long dsRNAs are first expressed from a promoter (such as CMV (pol II)) in the nuclei of selected cell lines or transgenic mice. The long dsRNAs are processed into siRNAs in the nuclei (e.g., by Dicer). The siRNAs exit from the nuclei and mediate gene-specific silencing. A similar approach can be used in conjunction with tissue-specific (pol II) promoters to create tissue-specific knockdown mice.

Any 3′ dinucleotide overhang, such as UU, can be used for siRNA design. In some cases, G residues in the overhang are avoided because of the potential for the siRNA to be cleaved by RNase at single-stranded G residues.

With regard to the siRNA sequence itself, it has been found that siRNAs with 30-50% GC content can be more active than those with a higher G/C content in certain cases. Moreover, since a 4-6 nucleotide poly(T) tract may act as a termination signal for RNA pol III, stretches of >4 Ts or As in the target sequence may be avoided in certain cases when designing sequences to be expressed from an RNA pol III promoter. In addition, some regions of mRNA may be either highly structured or bound by regulatory proteins. Thus, it may be helpful to select siRNA target sites at different positions along the length of the gene sequence. Finally, the potential target sites can be compared to the appropriate genome database (human, mouse, rat, etc.). Any target sequences with more than 16-17 contiguous base pairs of homology to other coding sequences may be eliminated from consideration in certain cases.

In one embodiment, siRNA can be designed to have two inverted repeats separated by a short spacer sequence and end with a string of Ts that serve as a transcription termination site. This design produces an RNA transcript that is predicted to fold into a short hairpin RNA (shRNA, e.g., SEQ ID NO: 7, which demonstrated herein down-regulates Ccdc80 mRNA in both undifferentiated 3T3-L1 cells and terminally differentiated 3T3-L1 adipocytes (see Example 3) and attenuates the ability of 3T3-L1 cells to differentiate into mature adipocytes (see Example 4)). The selection of siRNA target sequence, the length of the inverted repeats that encode the stem of a putative hairpin, the order of the inverted repeats, the length and composition of the spacer sequence that encodes the loop of the hairpin, and the presence or absence of 5′-overhangs, can vary to achieve desirable results.

siRNA targets can be selected by scanning an mRNA sequence for AA dinucleotides and recording the 19 nucleotides immediately downstream of the AA. Other methods can also been used to select the siRNA targets. In one example, the selection of the siRNA target sequence is purely empirically determined (see, e.g., Sui G et al., Proc. Natl. Acad. Sci. USA 99:5515-20 (2002)), as long as the target sequence starts with GG and does not share significant sequence homology with other genes as analyzed by BLAST search. In another example, a more elaborate method is employed to select the siRNA target sequences. This procedure exploits an observation that any accessible site in endogenous mRNA can be targeted for degradation by synthetic oligodeoxyribonucleotide/RNase H method (see, e.g., Lee N S et al., Nature Biotechnol. 20:500-05 (2002)).

In another embodiment, the hairpin siRNA expression cassette is constructed to contain the sense strand of the target, followed by a short spacer, the antisense strand of the target, and 5-6 Ts as transcription terminator. The order of the sense and antisense strands within the siRNA expression constructs can be altered without affecting the gene silencing activities of the hairpin siRNA. In certain instances, the reversal of the order may cause partial reduction in gene silencing activities.

The length of nucleotide sequence being used as the stem of siRNA expression cassette can range, for instance, from 19 to 29. The loop size can range from 3 to 23 nucleotides. Other lengths and/or loop sizes can also be used.

In yet another embodiment, a 5′ overhang in the hairpin siRNA construct can be used, provided that the hairpin siRNA is functional in gene silencing. In one specific example, the 5′ overhang includes about 6 nucleotide residues.

In still yet another embodiment, the target sequence for RNAi is a 21-mer sequence fragment. The 5′ end of the target sequence has dinucleotide “NA”, where “N” can be any base and “A” represents adenine. The remaining 19-mer sequence has a GC content of between 35% and 55%. In addition, the remaining 19-mer sequence does not include any four consecutive A or T (i.e., AAAA or TTTT), three consecutive G or C (i.e., GGG or CCC), or seven “GC” in a row.

Additional criteria can also be used for selecting RNAi target sequences. For instance, the GC content of the remaining 19-mer sequence can be limited to between 45% and 55%. Moreover, any 19-mer sequence having three consecutive identical bases (i.e., GGG, CCC, TTT, or AAA) or a palindrome sequence with 5 or more bases is excluded. Furthermore, the remaining 19-mer sequence can be selected to have low sequence homology to other genes. In one specific example, potential target sequences are searched by BLASTN against NCBI's human UniGene cluster sequence database. The human UniGene database contains non-redundant sets of gene-oriented clusters. Each UniGene cluster includes sequences that represent a unique gene. 19-mer sequences producing no hit to other human genes under the BLASTN search can be selected. During the search, the e-value may be set at a stringent value (such as “1”)

The effectiveness of the siRNA sequences, as well as any other RNAi sequence derived according to the present invention, can be evaluated using various methods known in the art. For instance, an siRNA sequence of the present invention can be introduced into a cell that expresses the Ccdc80 gene. The polypeptide or mRNA level of the Ccdc80 gene in the cell can be detected. A substantial change in the expression level of the Ccdc80 gene before and after the introduction of the siRNA sequence is indicative of the effectiveness of the siRNA sequence in suppressing the expression of the Ccdc80 gene. In one specific example, the expression levels of other genes are also monitored before and after the introduction of the siRNA sequence. An siRNA sequence which has inhibitory effect on Ccdc80 gene expression but does not significantly affect the expression of other genes can be selected. In another specific example, multiple siRNA or other RNAi sequences can be introduced into the same target cell. These siRNA or RNAi sequences specifically inhibit Ccdc80 gene expression but not the expression of other genes. In yet another specific example, siRNA or other RNAi sequences that inhibit the expression of the Ccdc80 gene and other gene or genes can be used.

Antisense polynucleotides may be produced from a heterologous expression cassette in a transfectant cell or transgenic cell. Alternatively, the antisense polynucleotides may comprise soluble oligonucleotides that are administered to the external milieu, either in the culture medium in vitro or in the circulatory system or in interstitial fluid in vivo. Soluble antisense polynucleotides present in the external milieu have been shown to gain access to the cytoplasm and inhibit translation of specific mRNA species.

### III. ISOLATED Ccdc80 PROTEINS AND FRAGMENTS THEREOF

Native Ccdc80 proteins can be isolated from cells or tissue sources by an appropriate purification scheme using standard protein purification techniques. In another embodiment, Ccdc80 proteins are produced by recombinant DNA techniques. Alternative to recombinant expression, a Ccdc80 protein or polypeptide can be synthesized chemically using standard peptide synthesis techniques. It will be understood that in discussing the uses of Ccdc80 proteins, e.g., human, mouse, rat, or chicken Ccdc80 (GenBank® accession numbers NM_{—}199511, NM_{—}026439, NM_{—}022543, NM_{—}204431), that fragments of such proteins that are not full-length Ccdc80 polypeptides as well as full-length Ccdc80 proteins can be used.

In a preferred embodiment, a Ccdc80 protein comprises the amino acid sequence of any of the aforementioned GenBank® sequences or a portion thereof. In other embodiments, a Ccdc80 protein has at least 65%, at least 70% amino acid identity, at least 80% amino acid identity, at least 85% amino acid identity, at least 90% amino acid identity, or at least 95% amino acid identity with the amino acid sequence shown in of any of the aforementioned GenBank® sequences portion thereof. Preferred portions of Ccdc80 polypeptide molecules are biologically active, for example, a portion of the Ccdc80 polypeptide having the ability to modulate preadipocyte proliferation and/or lipid accumulation.

Biologically active portions of a Ccdc80 protein include peptides comprising amino acid sequences sufficiently homologous to or derived from the amino acid sequence of the Ccdc80 protein, which include less amino acids than the full-length Ccdc80 proteins, and exhibit at least one activity of a Ccdc80 protein.

The invention also provides Ccdc80 chimeric or fusion proteins. For example, in one embodiment, the fusion protein is a GST-Ccdc80 member fusion protein in which the Ccdc80 member sequences are fused to the C-terminus of the GST sequences. In another embodiment, the fusion protein is a Ccdc80-HA fusion protein in which the Ccdc80 member polynucleotide sequence is inserted in a vector such as pCEP4-HA vector (Herrscher R F et al., Genes Dev. 9:3067-82 (1995)) such that the Ccdc80 member sequences are fused in frame to an influenza hemagglutinin epitope tag. In a further embodiment, the fusion protein may be an Fc-fusion protein. For example, a useful Fc fusion protein may be a chimeric protein consisting of Ccdc80 fused to the Fc region of an immunoglobulin G (IgG). The fusion can occur at either the N- or C-terminus of the Fc region. The Fc fusion protein may be expressed in cells using an expression plasmid. The resulting Fc fusion protein can be secreted into culture medium. For example, in some embodiments, the Fc region of immunoglobulin may be used as the N-terminal fusion partner, which can direct the cellular processes into expressing and secreting high levels of many different types of proteins, including, but not limited to, secreted proteins, such as Ccdc80.

Such fusion proteins can facilitate the purification of a recombinant Ccdc80 member. For example, with respect to Fc-fusion proteins, the Fc region provides for easy detection and purification. In particular, Fc-fusion proteins can be purified in a single-step using protein A or protein G affinity chromatography according to methods well known in the art. Protein A and protein G bind specifically to the Fc region of IgG. With respect to Fc-fusion proteins, the Fc region also provides for improved pharmaceutical properties (e.g., altered half-life and effector functions), and may be used as a therapeutic.

Fusion proteins and peptides produced by recombinant techniques may be secreted and isolated from a mixture of cells and medium containing the protein or peptide. Alternatively, the protein or peptide may be retained cytoplasmically and the cells harvested, lysed, and the protein isolated. A cell culture typically includes host cells, media, and other byproducts. Suitable media for cell culture are well known in the art. Protein and peptides can be isolated from cell culture media, host cells, or both using techniques known in the art for purifying proteins and peptides. Techniques for transfecting host cells and purifying proteins and peptides are known in the art.

In one embodiment, a Ccdc80 fusion protein is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al., John Wiley & Sons: 1992). Moreover, many expression vectors are commercially available that already encode a fusion moiety (e.g., a GST polypeptide or an HA epitope tag). A Ccdc80-encoding nucleic acid can be cloned into such an expression vector such that the fusion moiety is linked in-frame to the Ccdc80 protein.

In another embodiment, the fusion protein is a Ccdc80 protein containing a heterologous signal sequence at its N-terminus. In certain host cells (e.g., mammalian host cells), expression and/or secretion of Ccdc80 can be increased through use of a heterologous signal sequence. The Ccdc80 fusion proteins of the invention can be incorporated into pharmaceutical compositions and administered to a subject in vivo. Ccdc80 fusion proteins may be useful therapeutically for the treatment of obesity, insulin resistance, and/or type 2 diabetes.

The present invention also pertains to variants of Ccdc80 proteins which function as Ccdc80 agonists (mimetics). Variants of Ccdc80 proteins can be generated by mutagenesis, for example, discrete point mutation or truncation of a Ccdc80 protein. An agonist of a Ccdc80 protein can retain substantially the same, or a subset, of the biological activities of the naturally occurring form of a Ccdc80 protein. An antagonist of a Ccdc80 protein can inhibit one or more of the activities of the naturally occurring form of a Ccdc80 protein by, for example, competitively modulating a cellular activity of a Ccdc80 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of a Ccdc80 protein.

In one embodiment, the invention pertains to derivatives of Ccdc80 which may be formed by modifying at least one amino acid residue of Ccdc80 by oxidation, reduction, or other derivatization processes known in the art.

In one embodiment, variants of a Ccdc80 protein which function as Ccdc80 agonists (mimetics) can be identified by screening combinatorial libraries of mutants, for example, truncation mutants, of a Ccdc80 protein for Ccdc80 protein agonist activity. In one embodiment, a variegated library of Ccdc80 variants is generated by combinatorial mutagenesis at the nucleic acid level and is encoded by a variegated gene library. A variegated library of Ccdc80 variants can be produced by, for example, enzymatically ligating a mixture of synthetic oligonucleotides into gene sequences such that a degenerate set of potential Ccdc80 sequences is expressible as individual polypeptides, or alternatively, as a set of larger fusion proteins (e.g., for phage display) containing the set of Ccdc80 sequences therein. There are a variety of methods which can be used to produce libraries of potential Ccdc80 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential Ccdc80 sequences. Methods for synthesizing degenerate oligonucleotides are known in the art (see, e.g., Narang S A, Tetrahedron 39:3-22 (1983); Itakura K et al., Annu. Rev. Biochem. 53:323-56 (1984); Itakura K et al., Science 198:1056-63 (1977); Ike Y et al., Nucleic Acids Res. 11:477-88 (1983)).

In addition, libraries of fragments of a Ccdc80 protein coding sequence can be used to generate a variegated population of Ccdc80 fragments for screening and subsequent selection of variants of a Ccdc80 protein. In one embodiment, a library of coding sequence fragments can be generated by treating a double stranded PCR fragment of a Ccdc80 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with SI nuclease, and ligating the resulting fragment library into an expression vector. By this method, an expression library can be derived which encodes N-terminal, C-terminal, and internal fragments of various sizes of a Ccdc80 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of Ccdc80 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify Ccdc80 variants (Arkin A P and Youvan D C, Proc. Natl. Acad. Sci. USA 89:7811-15 (1992); Delgrave S et al., Protein Eng. 6:327-31 (1993)).

In one embodiment, cell based assays can be exploited to analyze a variegated Ccdc80 library. For example, a library of expression vectors can be transfected into a cell line which ordinarily synthesizes and secretes Ccdc80. The transfected cells are then cultured such that Ccdc80 and a particular mutant Ccdc80 are secreted and the effect of expression of the mutant on Ccdc80 activity in cell supernatants can be detected, for example, by any of a number of enzymatic assays. Plasmid DNA can then be recovered from the cells which score for inhibition, or alternatively, potentiation of Ccdc80 activity, and the individual clones further characterized.

In addition to Ccdc80 polypeptides consisting only of naturally-occurring amino acids, Ccdc80 peptidomimetics are also useful. Peptide analogs are commonly used in the pharmaceutical industry as non-peptide drugs with properties analogous to those of the template peptide. These types of non-peptide compound are termed “peptide mimetics” or “peptidomimetics” (Fauchere J, Adv. Drug Res. 15:29 (1986); Veber D F and Freidinger R M, Trends Neurosci. 8:392-96 (1985); Evans B E et al., J. Med. Chem. 30:1229-39 (1987)) and are usually developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biological or pharmacological activity), such as human Ccdc80, but have one or more peptide linkages optionally replaced by a linkage selected from the group consisting of: —CH₂NH—, —CH₂S—, —CH₂CH₂—, —CH═CH—(cis and trans), —COCH₂—, —CH(OH)CH₂—, and —CH₂SO—, by methods known in the art and further described in the following references: Spatola A F in “Chemistry and Biochemistry of Amino Acids, Peptides, and Proteins,” B. Weinstein, ed., Marcel Dekker, New York, p. 267 (1983); Spatola, A F, Vega Data (March 1983), Vol. 1, Issue 3, “Peptide Backbone Modifications” (general review); Morley J S, Trends Pharmcol. Sci. 1:463-68 (1980) (general review); Hudson D et al., Int. J. Pept. Prot. Res. 14:177-85 (1979) (—CH₂NH—, CH₂CH₂—); Spatola A F et al., Life Sci. 38:1243-49 (1986) (—CH₂S—); Hann M M, J. Chem. Soc. Perkin Trans. 1, 307-314 (1982) (—CH═CH—, cis and trans); Almquist R G et al., J. Med. Chem. 23:1392-98 (1980) (—COCH₂—); Jennings-White C et al., Tetrahedron Lett. 23:2533-34 (1982) (—COCH₂—); EP 0 045 665 (—CH(OH)CH₂—); Holladay M W et al., Tetrahedron Lett., 24:4401-04 (1983) (—C(OH)CH₂—); Hruby V J, Life Sci. 31:189-99 (1982) (—CH₂S—). A particularly preferred non-peptide linkage is —CH₂NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) to which the peptidomimetic binds to produce the therapeutic effect. Derivatization (e.g., labeling) of peptidomimetics should not substantially interfere with the desired biological or pharmacological activity of the peptidomimetic.

Systematic substitution of one or more amino acids of a Ccdc80 amino acid sequence with a D-amino acid of the same type (e.g., D-lysine in place of L-lysine) may be used to generate more stable peptides. In addition, constrained peptides comprising a Ccdc80 amino acid sequence or a substantially identical sequence variation may be generated by methods known in the art (Rizo J and Gierasch L M, Ann. Rev. Biochem. 61:387-416 (1992)); for example, by adding internal cysteine residues capable of forming intramolecular disulfide bridges which cyclize the peptide.

Amino acid sequences of Ccdc80 polypeptides will enable those of skill in the art to produce polypeptides corresponding to Ccdc80 peptide sequences and sequence variants thereof. Such polypeptides may be produced in prokaryotic or eukaryotic host cells by expression of polynucleotides encoding a Ccdc80 peptide sequence, frequently as part of a larger polypeptide. Alternatively, such peptides may be synthesized by chemical methods. Methods for expression of heterologous proteins in recombinant hosts, chemical synthesis of polypeptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Gutte B and Merrifield R B, J. Am. Chem. Soc. 91:501-02 (1969); Chaiken I M, CRC Crit. Rev. Biochem. 11:255-301 (1981); Kaiser E T et al., Science 243:187-92 (1989); Merrifield B, Science 232:341-47 (1986); Kent S B H, Ann. Rev. Biochem. 57:957-89 (1988); Offord, R. E. (1980) Semisynthetic Proteins, Wiley Publishing.

Peptides can be produced, for example, by direct chemical synthesis. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, may be incorporated into various embodiments of the invention. Certain amino-terminal and/or carboxy-terminal modifications and/or peptide extensions to the core sequence can provide advantageous physical, chemical, biochemical, and pharmacological properties such as, for example, enhanced stability, increased potency and/or efficacy, resistance to serum proteases, desirable pharmacokinetic properties, and others. Peptides may be used therapeutically to treat disease.

An isolated Ccdc80 protein, or a portion or fragment thereof, can also be used as an immunogen to generate antibodies that bind Ccdc80 using standard techniques for polyclonal and monoclonal antibody preparation. A full-length Ccdc80 protein can be used or, alternatively, the invention provides antigenic peptide fragments of Ccdc80 for use as immunogens. The antigenic peptide of Ccdc80 comprises at least 8 amino acid residues and encompasses an epitope of Ccdc80 such that an antibody raised against the peptide forms a specific immune complex with Ccdc80. In other embodiments, the antigenic peptide comprises at least 10 amino acid residues, at least 15 amino acid residues, at least 20 amino acid residues, or at least 30 amino acid residues.

In one embodiment, epitopes encompassed by the antigenic peptide are regions of a Ccdc80 polypeptide that are located on the surface of the protein, for example, hydrophilic regions, and that are unique to a Ccdc80 polypeptide. In one embodiment, such epitopes can be specific for a Ccdc80 protein from one species, such as mouse or human (i.e., an antigenic peptide that spans a region of a Ccdc80 polypeptide that is not conserved across species is used as immunogen; such non-conserved residues can be determined using an alignment program such as that described herein). A standard hydrophobicity analysis of the protein can be performed to identify hydrophilic regions.

A Ccdc80 immunogen typically is used to prepare antibodies by immunizing a suitable subject (e.g., rabbit, goat, mouse, or other mammal) with the immunogen. An appropriate immunogenic preparation can contain, for example, a recombinantly expressed Ccdc80 protein or a chemically synthesized Ccdc80 peptide. The preparation can further include an adjuvant, such as Freund's complete or incomplete adjuvant, or similar immunostimulatory agent. Immunization of a suitable subject with an immunogenic Ccdc80 preparation induces a polyclonal anti-Ccdc80 antibody response.

Accordingly, another aspect pertains to the use of anti-Ccdc80 antibodies. Polyclonal anti-Ccdc80 antibodies can be prepared as described above by immunizing a suitable subject with a Ccdc80 immunogen. The anti-Ccdc80 antibody titer in the immunized subject can be monitored over time by standard techniques, such as with an enzyme linked immunosorbent assay (ELISA) using immobilized Ccdc80 polypeptide. If desired, the antibody molecules directed against a Ccdc80 polypeptide can be isolated from the mammal (e.g., from the blood) and further purified by well known techniques, such as protein A chromatography to obtain the IgG fraction. At an appropriate time after immunization, for example, when the anti-Ccdc80 antibody titers are highest, antibody-producing cells can be obtained from the subject and used to prepare monoclonal antibodies by standard techniques, such as the hybridoma technique originally described by Kohler G and Milstein C, Nature 256:495-97 (1975) (see also, Brown J P et al., J. Immunol. 127:539-46 (1981); Brown J P et al., J. Biol. Chem. 255:4980-83 (1980); Yeh M Y et al., Proc. Natl. Acad. Sci. USA 76:2927-31 (1979); Yeh M Y et al., Int. J. Cancer 29:269-75 (1982)), the more recent human B cell hybridoma technique (Kozbor D and Roder J C, Immunol. Today 4:72-79 (1983)), the EBV-hybridoma technique (Cole et al. (1985), Monoclonal Antibodies and Cancer Therapy, Alan R. Liss, Inc., pp. 77-96), or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); Lerner E A, Yale J. Biol. Med., 54:387-402 (1981); Gefter M L et al., Somatic Cell Genet. 3:231-36 (1977)). Briefly, an immortal cell line (typically a myeloma) is fused to lymphocytes (typically splenocytes) from a mammal immunized with a Ccdc80 immunogen as described above, and the culture supernatants of the resulting hybridoma cells are screened to identify a hybridoma producing a monoclonal antibody that binds specifically to a Ccdc80 polypeptide.

Any of the many well known protocols used for fusing lymphocytes and immortalized cell lines can be applied for the purpose of generating an anti-Ccdc80 monoclonal antibody (see, e.g., Galfre G et al., Nature 266:550-52 (1977); Geifer M L et al., supra; Lerner E A, supra; Kenneth, Monoclonal Antibodies, supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines may be used as a fusion partner according to standard techniques, for example, the P3-NS1/1-Ag4-1, P3-x63-Ag8.653 or Sp2/0-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a Ccdc80 molecule, for example, using a standard ELISA assay.

As an alternative to preparing monoclonal antibody-secreting hybridomas, a monoclonal anti-Ccdc80 antibody can be identified and isolated by screening a recombinant combinatorial immunoglobulin library (e.g., an antibody phage display library) with Ccdc80 to thereby isolate immunoglobulin library members that bind a Ccdc80 polypeptide. Kits for generating and screening phage display libraries are commercially available (e.g., the GE Healthcare Recombinant Phage Antibody System, Catalog No. 27-9400-01). Additionally, examples of methods and reagents particularly amenable for use in generating and screening antibody display library can be found in, for example, U.S. Pat. No. 5,223,409; WO 92/18619; WO 91/17271; WO 92/20791; WO 92/15679; WO 93/01288; WO 92/01047; WO 92/09690; WO 90/02809; Fuchs P et al., Biotechnology (N.Y.) 9:1370-72 (1991); Hay B N et al., Hum. Antibodies Hybridomas 3:81-85 (1992); Huse W D et al., Science 246:1275-81 (1989); Griffiths A D et al., EMBO J. 12:725-34 (1993); Hawkins R E et al., J. Mol. Biol. 226:889-96 (1992); Clarkson T et al., Nature 352:624-28 (1991); Gram H et al., Proc. Natl. Acad. Sci. USA 89:3576-80 (1992); Garrard L J et al., Biotechnology (N.Y.) 9:1373-77 (1991); Hoogenboom H R et al., Nucleic Acids Res. 19:4133-37 (1991); Barbas C F et al., Proc. Natl. Acad. Sci. USA 88:7978-82 (1991); and McCafferty J et al., Nature 348:552-54 (1990).

Additionally, recombinant anti-Ccdc80 antibodies, such as chimeric and humanized monoclonal antibodies, comprising both human and non-human portions, can be produced by recombinant DNA techniques known in the art, for example using methods described in WO 87/02671; EP 0 184 187; EP 0 171 496; EP 0 173 494; WO 86/01533; U.S. Pat. No. 4,816,567; EP 0 125 023; Better M et al., Science 240:1041-43 (1988); Liu A Y et al., Proc. Natl. Acad. Sci. USA 84:3439-43 (1987); Liu A Y et al., J. Immunol. 139:3521-26 (1987); Sun L K et al., Proc. Natl. Acad. Sci. USA 84:214-18 (1987); Nishimura Y et al., Cancer Res. 47:999-1005 (1987); Wood C R et al., Nature 314:446-49 (1985); Shaw D R et al., J. Natl. Cancer Inst. 80:1553-59 (1988); Morrison S L, Science 229:1202-07 (1985); U.S. Pat. No. 5,225,539; Verhocyan M et al., Science 239:1534-36 (1988); and Beidler C B et al., J. Immunol. 141:4053-60 (1988).

In addition, humanized antibodies can be made according to standard protocols such as those disclosed in U.S. Pat. No. 5,565,332. In another embodiment, antibody chains or specific binding pair members can be produced by recombination between vectors comprising nucleic acid molecules encoding a fusion of a polypeptide chain of a specific binding pair member and a component of a replicable genetic display package and vectors containing nucleic acid molecules encoding a second polypeptide chain of a single binding pair member using techniques known in the art, for example, as described in U.S. Pat. No. 5,565,332; 5,871,907; or 5,733,743.

An anti-Ccdc80 antibody (e.g., monoclonal antibody) can be used to isolate a Ccdc80 polypeptide by standard techniques, such as affinity chromatography or immunoprecipitation. Anti-Ccdc80 antibodies can facilitate the purification of natural Ccdc80 polypeptides from cells and of recombinantly produced Ccdc80 polypeptides expressed in host cells. Moreover, an anti-Ccdc80 antibody can be used to detect a Ccdc80 protein (e.g., in a cellular lysate or cell supernatant). Detection may be facilitated by coupling (i.e., physically linking) the antibody to a detectable substance. Accordingly, in one embodiment, an anti-Ccdc80 antibody of the invention is labeled with a detectable substance. Examples of detectable substances include various enzymes, prosthetic groups, fluorescent materials, luminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, or phycoerythrin; an example of a luminescent material includes luminol; and examples of suitable radioactive material include ¹²⁵I, ¹³¹I, ³⁵S, or ³H.

In some embodiments, antibodies that recognize extracellular Ccdc80 are used to inhibit Ccdc80 protein activity. For example, to produce soluble (secreted) Ccdc80 protein, a Ccdc80-Fc fusion protein may be generated by PCR, sequenced, and cloned into an expression vector, and then transfected into cells, such as CHO cells. The soluble Ccdc80-Fc fusion protein is secreted into the culture medium by the transfected cells, and then purified from the culture medium by using, for example, protein A chromatography according to methods well known in the art. Subjects, such as rabbits, rats or mice, may then be immunized with purified Ccdc80-Fc fusion protein mixed with an adjuvant. The anti-Ccdc80 antibody titer in the sera of the immunized subject(s) can be monitored over time by standard techniques, such as with an enzyme linked immunosorbent assay (ELISA) using an immobilized Ccdc80 polypeptide.

Polyclonal antibody molecules directed against the extracellular Ccdc80 polypeptide can be isolated from the immunized mammal (e.g., from the sera) and further purified by well known techniques, such as protein A chromatography to obtain the IgG fraction. Alternatively, an anti-Ccdc80 monoclonal antibody may be generated. For example, cells from the spleens of the immunized subjects having the highest anti-Ccdc80 specific response may be used to prepare monoclonal antibodies by standard techniques, such as the hybridoma technique originally described by Kohler G and Milstein C, Nature 256:495-97 (1975). Polyclonal or monoclonal antibodies that recognize extracellular Ccdc80, or an extracellular domain thereof, may be used to inhibit the functional activity of extracellular Ccdc80 protein.

In a further embodiment, anti-Ccdc80 antibodies that recognize intracellular Ccdc80 can be used, e.g., intracellularly to inhibit Ccdc80 protein activity. The use of intracellular antibodies to inhibit protein function in a cell is known in the art (see e.g., Carlson J R, Mol. Cell. Biol. 8:2638-46 (1988); Biocca S et al., EMBO J. 9:101-08 (1990); Werge T M et al., FEBS Lett. 274:193-98 (1990); Carlson J R, Proc. Natl. Acad. Sci. USA 90:7427-28 (1993); Marasco W A et al., Proc. Natl. Acad. Sci. USA 90:7889-93 (1993); Biocca S et al., Biotechnology (N.Y.) 12:396-99 (1994); Chen S-Y et al., Hum. Gene Ther. 5:595-601 (1994); Duan L et al., Proc. Natl. Acad. Sci. USA 91:5075-79 (1994); Chen S-Y et al., Proc. Natl. Acad. Sci. USA 91:5932-36 (1994); Beerli R R et al., J. Biol. Chem. 269:23931-36 (1994); Beerli R R et al., Biochem. Biophys. Res. Commun. 204:666-72 (1994); Mhashilkar A M et al., EMBO J. 14:1542-51 (1995); Richardson J H et al., Proc. Natl. Acad. Sci. USA 92:3137-41 (1995); WO 94/02610; and WO 95/03832).

In one embodiment, a recombinant expression vector is prepared which encodes the antibody chains in a form such that, upon introduction of the vector into a cell, the antibody chains are expressed by the cell as a functional antibody. For inhibition of secreted Ccdc80 activity, an antibody that specifically binds to Ccdc80 preferably recognizes extracellular Ccdc80, and is secreted from the cell. For example, an expression plasmid may be used to facilitate the generation of an Fc-fusion protein where the fusion protein is a chimeric protein consisting of the Fab region of the anti-Ccdc80 antibody fused to the Fc region of an immunoglobulin G (IgG). The Fc region provides a handle for detection of the antibody. In some further embodiments, the antibody expressed by the cell may recognize intracellular Ccdc80. For inhibition of Ccdc80 activity according to the inhibitory methods of the invention, an intracellular antibody that specifically binds Ccdc80 protein is preferably secreted from the cell.

To prepare an antibody expression vector, antibody light and heavy chain cDNAs encoding antibody chains specific for the target protein of interest, for example, Ccdc80, are isolated, typically from a hybridoma that secretes a monoclonal antibody specific for the Ccdc80 protein. Hybridomas secreting anti-Ccdc80 monoclonal antibodies, or recombinant anti-Ccdc80 monoclonal antibodies, can be prepared as described above. Once a monoclonal antibody specific for Ccdc80 protein has been identified (e.g., either a hybridoma-derived monoclonal antibody or a recombinant antibody from a combinatorial library), DNAs encoding the light and heavy chains of the monoclonal antibody are isolated by standard molecular biology techniques. For hybridoma derived antibodies, light and heavy chain cDNAs can be obtained, for example, by PCR amplification or cDNA library screening. For recombinant antibodies, such as from a phage display library, cDNA encoding the light and heavy chains can be recovered from the display package (e.g., phage) isolated during the library screening process. Nucleotide sequences of antibody light and heavy chain genes from which PCR primers or cDNA library probes can be prepared are known in the art. For example, many such sequences are disclosed in Kabat E A et al. (1991) Sequences of Proteins of Immunological Interest, Fifth Edition, U.S. Department of Health and Human Services, NIH Publication No. 91-3242 and in the “Vbase” human germline sequence database.

Once obtained, the antibody light and heavy chain sequences are cloned into a recombinant expression vector using standard methods. An antibody expression vector can encode an antibody in one of several different forms. For example, in one embodiment, the vector encodes full-length antibody light and heavy chains such that a full-length antibody is expressed. To inhibit Ccdc80 activity in a cell, the expression vector encoding the anti-Ccdc80 intracellular or extracellular antibody is introduced into the cell by standard transfection methods, as discussed herein.

### IV. RECOMBINANT EXPRESSION VECTORS AND HOST CELLS

Recombinant expression vectors can comprise a nucleic acid in a form suitable for expression of the nucleic acid in a host cell, which means that the recombinant expression vectors include one or more regulatory sequences, selected on the basis of the host cells to be used for expression, that are operably linked to the nucleic acid sequence to be expressed. The term “regulatory sequence” is intended to include promoters, enhancers, and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., Ccdc80 proteins, mutant forms of Ccdc80 proteins, fusion proteins, and the like).

Recombinant expression vectors can be designed for expression of proteins or protein fragments in prokaryotic or eukaryotic cells. For example, Ccdc80 proteins can be expressed in bacterial cells such as E. coli, insect cells (using baculovirus expression vectors), yeast cells, or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin, and enterokinase. Typical fusion expression vectors include, for example, pGEX (Pharmacia Biotech Inc.; Smith D B and Johnson K S, Gene 67:31-40 (1988)) and pMAL (New England Biolabs, Beverly, Mass.) which fuse glutathione S-transferase (GST) or maltose E binding protein, respectively, to the target recombinant protein.

Examples of suitable inducible non-fusion E. coli expression vectors include pTrc (Amann E et al., Gene 69:301-15 (1988)) and pET 11d (Studier et al., Gene Expression Technology Methods in Enzymology 185, Academic Press, San Diego, Calif. (1990) pp. 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman S, Gene Expression Technology: Methods in Enzymology 185, Academic Press, San Diego, Calif. (1990) pp. 119-28). Another strategy is to alter the nucleic acid sequence of the nucleic acid to be inserted into an expression vector so that the individual codons for each amino acid are those preferentially utilized in E. coli (Wada K et al., Nucleic Acids Res. 20(Suppl.):2111-18 (1992)). Such alteration of nucleic acid sequences of the invention can be carried out by standard DNA synthesis techniques.

In another embodiment, the expression vector is a yeast expression vector. Examples of vectors for expression in yeast S. cerevisiae include pYepSec1 (Baldari C et al., EMBO J. 6:229-34 (1987)), pMFa (Kurjan J and Herskowitz I, Cell 30:933-43 (1982)), pJRY88 (Schultz L D et al., Gene 54:113-23 (1987)), pYES2 (Invitrogen Corp., San Diego, Calif.), and picZ (Invitrogen Corp).

Alternatively, proteins or polypeptides can be expressed in insect cells using baculovirus expression vectors. Baculovirus vectors available for expression of proteins in cultured insect cells (e.g., Sf 9 cells) include the pAc series (Smith G E et al., Mol. Cell. Biol. 3:2156-65 (1983)) and the pVL series (Lucklow V A and Summers M D, Virology 170:31-39 (1989)).

In yet another embodiment, nucleic acids are expressed in mammalian cells using a mammalian expression vector. Examples of mammalian expression vectors include pCDM8 (Seed B, Nature 329:840-41 (1987)) and pMT2PC (Kaufman R J et al., EMBO J. 6:187-95 (1987)). When used in mammalian cells, the expression vector's control functions are often provided by viral regulatory elements. For example, commonly used promoters are derived from polyoma, Adenovirus 2, cytomegalovirus, and Simian Virus 40. For other suitable expression systems for both prokaryotic and eukaryotic cells, see chapters 16 and 17 of Sambrook, J., Fritsh, E. F., and Maniatis, T. Molecular Cloning: A Laboratory Manual, 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert C A et al., Genes Dev. 1:268-77 (1987)), lymphoid-specific promoters (Calame K and Eaton S, Adv. Immunol. 43:235-75 (1988)), in particular promoters of T cell receptors (Winoto A and Baltimore D, EMBO J. 8:729-33 (1989)) and immunoglobulins (Banerji J et al., Cell 33:729-40 (1983); Queen C and Baltimore D, Cell 33:741-48 (1983)), neuron-specific promoters (e.g., the neurofilament promoter; Byrne G W and Ruddle F H, Proc. Natl. Acad. Sci. USA 86:5473-77 (1989)), pancreas-specific promoters (Edlund T et al., Science 230:912-16 (1985)), and mammary gland-specific promoters (e.g., milk whey promoter; U.S. Pat. No. 4,873,316 and EP 0 264 166). Developmentally-regulated promoters are also encompassed, for example the murine hox promoters (Kessel M and Gruss P, Science 249:374-79 (1990)) and the α-fetoprotein promoter (Camper S A and Tilghman S M, Genes Dev. 3:537-46 (1989)).

Moreover, inducible regulatory systems for use in mammalian cells are known in the art, for example systems in which gene expression is regulated by heavy metal ions (see e.g., Mayo K E et al., Cell 29:99-108 (1982); Brinster R L et al., Nature 296:39-42 (1982); Searle P F et al., Mol. Cell. Biol. 5:1480-89 (1985)), heat shock (see e.g., Nouer L et al. (1991) in Heat Shock Response, ed. Nouer L, CRC, Boca Raton, Fla., pp. 167-220), hormones (see e.g., Lee F et al., Nature 294:228-32 (1981); Hynes N E et al., Proc. Natl. Acad. Sci. USA 78:2038-42 (1981); Klock G et al., Nature 329:734-36 (1987); Israel D I and Kaufman R J, Nucleic Acids Res. 17:2589-2604 (1989); WO 93/23431), FK506-related molecules (see e.g., WO 94/18317) or tetracyclines (Gossen M and Bujard H, Proc. Natl. Acad. Sci. USA 89:5547-51 (1992); Gossen M et al., Science 268:1766-69 (1995); WO 94/29442; WO 96/01313). Accordingly, in another embodiment, the invention provides a recombinant expression vector in which a DNA is operably linked to an inducible eukaryotic promoter, thereby allowing for inducible expression of a protein in eukaryotic cells.

Also known in the art are methods for expressing endogenous proteins using one-arm homologous recombination (see, e.g., U.S. Published Patent Application No. 2005/0003367; Zeh et al., Assay Drug Dev. Technol. 1:755-65 (2003); Qureshi et al., Assay Drug Dev. Technol. 1:767-76 (2003)). Briefly, an isolated genomic construct comprising a promoter operably linked to a targeting sequence is introducing into a homogeneous population of cells (such as, for example, a homogeneous population of a human cell line). The promoter is heterologous to the target gene. Following recombination, the promoter controls transcription of an mRNA that encodes a polypeptide. The population of cells is then incubated under conditions which cause expression of the polypeptide.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including, for example, calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual, 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin, and methotrexate. A nucleic acid molecule encoding a selectable marker can be introduced into a host cell on the same vector as that encoding a protein or can be introduced on a separate vector. Cells stably transfected with the introduced nucleic acid molecule can be identified by drug selection (e.g., cells that have incorporated the selectable marker gene will survive, while the other cells die).

In the case of, for example, HEK293, HEK293T, CHO, COS, C2C12, 3T3-L1, or msenchymal stem cells that are stably transfected with Ccdc80, such lines can be made such that the Ccdc80 gene is inducible, for example, using Tet-on/Tet-off systems.

### V. USES AND METHODS OF THE INVENTION

The Ccdc80 modulators described herein can be used in one or more of the following methods: a) methods of treatment, preferably in adipocytic cells; b) screening assays; c) predictive medicine (e.g., diagnostic assays, prognostic assays, monitoring clinical trials, or pharmacogenetics). The isolated nucleic acid molecules of the invention can be used, for example, to express Ccdc80 protein (e.g., via a recombinant expression vector in a host cell in gene therapy applications) and to modulate Ccdc80 activity, as described further below. In addition, the Ccdc80 proteins can be used to screen for naturally occurring Ccdc80 binding proteins, to screen for drugs or compounds which modulate Ccdc80 activity, as well as to treat disorders that would benefit from modulation of Ccdc80, for example, characterized by insufficient or excessive production of Ccdc80 protein or production of Ccdc80 protein forms which have decreased or aberrant activity compared to Ccdc80 wild type protein. In some embodiments, the methods of the invention, for example, detection, modulation, etc. of Ccdc80 are performed in adipocytic cells.

### A. Methods of Modulating Ccdc80

According to one modulatory method, Ccdc80 activity is stimulated in a cell by contacting the cell with a stimulatory agent. Examples of such stimulatory agents include active Ccdc80 protein and nucleic acid molecules encoding Ccdc80 that are introduced into the cell to increase Ccdc80 activity in the cell. To express a Ccdc80 protein in a cell, typically a Ccdc80 cDNA is first introduced into a recombinant expression vector using standard molecular biology techniques, as described herein. A Ccdc80 cDNA can be obtained, for example, by amplification using the PCR or by screening an appropriate cDNA library as described herein. Following isolation or amplification of Ccdc80 cDNA, the DNA fragment is introduced into an expression vector and transfected into target cells by standard methods, as described herein. Other stimulatory agents that can be used to stimulate the activity and/or expression of a Ccdc80 protein are chemical compounds that stimulate Ccdc80 activity and/or expression in cells, such as compounds that effect Ccdc80 modulation of preadipocyte proliferation and/or lipid accumulation. Such compounds can be identified using screening assays that select for such compounds, as described in detail herein.

Modulatory methods can be performed in vitro (e.g., by culturing the cell with the agent or by introducing the agent into cells in culture) or, alternatively, in vivo (e.g., by administering the agent to a subject or by introducing the agent into cells of a subject, such as by gene therapy). For practicing a modulatory method in vitro, cells can be obtained from a subject by standard methods and incubated (i.e., cultured) in vitro with a modulatory agent to modulate Ccdc80 activity in the cells. Ccdc80 modulators of adipogenesis may also be used to induce or inhibit differentiation of isolated preadipocytes or adipocytes in culture, for example 3T3-L1, 3T3 F422A, ob 1771, or preadipocytes and adipocytes from transgenic animals that can be induced to overexpress Ccdc80. It is within the skill of the artisan to administer Ccdc80 modulators to the isolated preadipocytes or adipocytes and to observe the differentiation of the in vitro cells (see, e.g., Example 4).

For agents that comprise nucleic acids (including recombinant expression vectors encoding Ccdc80 protein, antisense RNA, intracellular antibodies, or dominant negative inhibitors), the agents can be introduced into cells of the subject using methods known in the art for introducing nucleic acid (e.g., DNA) into cells in vivo. Examples of such methods encompass both non-viral and viral methods, including:

Direct Injection: Naked DNA can be introduced into cells in vivo by directly injecting the DNA into the cells (see, e.g., Acsadi G et al., Nature 332:815-18 (1991); Wolff J A et al., Science 247:1465-68 (1990)). For example, a delivery apparatus (e.g., a “gene gun”) for injecting DNA into cells in vivo can be used. Such an apparatus is commercially available (e.g., from Bio-Rad Laboratories, Hercules, Calif.).

Cationic Lipids: Naked DNA can be introduced into cells in vivo by complexing the DNA with cationic lipids or encapsulating the DNA in cationic liposomes. Examples of suitable cationic lipid formulations include N-[-1-(2,3-dioleoyloxy)propyl]N,N,N-triethylammonium chloride (DOTMA) and a 1:1 molar ratio of 1,2-dimyristyloxy-propyl-3-dimethylhydroxyethylammonium bromide (DMRIE) and dioleoyl phosphatidylethanolamine (DOPE) (see e.g., Logan J J et al., Gene Ther. 2:38-49 (1995); San H et al., Hum. Gene Ther. 4:781-88 (1993)).

Receptor-Mediated DNA Uptake: Naked DNA can also be introduced into cells in vivo by complexing the DNA to a cation, such as polylysine, which is coupled to a ligand for a cell-surface receptor (see, e.g., Wu G Y and Wu C H, J. Biol. Chem. 263:14621-24 (1988); Wilson J M et al., J. Biol. Chem. 267:963-67 (1992); and U.S. Pat. No. 5,166,320). Binding of the DNA-ligand complex to the receptor facilitates uptake of the DNA by receptor-mediated endocytosis. A DNA-ligand complex linked to adenovirus capsids which naturally disrupt endosomes, thereby releasing material into the cytoplasm can be used to avoid degradation of the complex by intracellular lysosomes (see, e.g., Curiel D T et al., Proc. Natl. Acad. Sci. USA 88:8850-54 (1991); Cristiano R J et al., Proc. Natl. Acad. Sci. USA 90:2122-26 (1993)).

Retroviruses: Defective retroviruses are well characterized for use in gene transfer for gene therapy purposes (for a review, see Miller A D, Blood 76:271-78 (1990)). A recombinant retrovirus can be constructed having a nucleotide sequence of interest incorporated into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel F M et al. (eds.) Greene Publishing Associates, (1989), Sections 9.10-9.14 and other standard laboratory manuals. Examples of suitable retroviruses include pLJ, pZIP, pWE, and pEM which are well known to those skilled in the art. Examples of suitable packaging virus lines include ψpCrip, ψpCre, ψp2 and ψpAm. Retroviruses have been used to introduce a variety of genes into many different cell types, including epithelial cells, endothelial cells, lymphocytes, myoblasts, hepatocytes, bone marrow cells, in vitro and/or in vivo (see, e.g., Eglitis M A et al., Science 230:1395-98 (1985); Danos O and Mulligan R C, Proc. Natl. Acad. Sci. USA 85:6460-64 (1988); Wilson J M et al., Proc. Natl. Acad. Sci. USA 85:3014-18 (1988); Armentano D et al., Proc. Natl. Acad. Sci. USA 87:6141-45 (1990); Huber B E et al., Proc. Natl. Acad. Sci. USA 88:8039-43 (1991); Ferry N et al., Proc. Natl. Acad. Sci. USA 88:8377-81 (1991); Chowdhury J R et al., Science 254:1802-05 (1991); van Beusechem V W et al., Proc. Natl. Acad. Sci. USA 89:7640-44 (1992); Kay M A et al., Hum. Gene Ther. 3:641-47 (1992); Dai Y et al., Proc. Natl. Acad. Sci. USA 89:10892-95 (1992); Hwu P et al., J. Immunol. 150:4104-15 (1993); U.S. Pat. No. 4,868,116; U.S. Pat. No. 4,980,286; WO 89/07136; WO 89/02468; WO 89/05345; and WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

Adenoviruses: The genome of an adenovirus can be manipulated such that it encodes and expresses a gene product of interest but is inactivated in terms of its ability to replicate in a normal lytic viral life cycle (see, e.g., Berkner K L, Biotechniques 6:616-29 (1988); Rosenfeld M A et al., Science 252:431-34 (1991); and Rosenfeld M A et al., Cell 68:143-55 (1992)). Suitable adenoviral vectors derived from the adenovirus strain Ad type 5 d1324 or other strains of adenovirus (e.g., Ad2, Ad3, Ad7, etc.) are well known to those skilled in the art. Recombinant adenoviruses are advantageous in that they do not require dividing cells to be effective gene delivery vehicles and can be used to infect a wide variety of cell types, including airway epithelium (Rosenfeld M A et al., Cell 68:143-55 (1992)), endothelial cells (Lemarchand P et al., Proc. Natl. Acad. Sci. USA 89:6482-86 (1992)), hepatocytes (Herz J and Gerard R D, Proc. Natl. Acad. Sci. USA 90:2812-16 (1993)), and muscle cells (Quantin B et al., Proc. Natl. Acad. Sci. USA 89:2581-84 (1992)). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner K L et al., supra; Haj-Ahmad Y and Graham F L, J. Virol. 57:267-74 (1986)). Most replication-defective adenoviral vectors currently in use are deleted for all or parts of the viral E1 and E3 genes but retain as much as 80% of the adenoviral genetic material.

Adeno-Associated Viruses: Adeno-associated virus (AAV) is a naturally occurring defective virus that requires another virus, such as an adenovirus or a herpes virus, as a helper virus for efficient replication and a productive life cycle (for a review, see Muzyczka N, Curr. Top. Microbiol. Immunol. 158:97-129 (1992)). It is also one of the few viruses that may integrate its DNA into non-dividing cells, and exhibits a high frequency of stable integration (see, e.g., Flotte T R et al., Am. J. Respir. Cell. Mol. Biol. 7:349-56 (1992); Samulski R J et al., J. Virol. 63:3822-28 (1989); and McLaughlin S K et al., J. Virol. 62:1963-73 (1988)). Vectors containing as little as 300 base pairs of AAV can be packaged and can integrate. Space for exogenous DNA is limited to about 4.5 kb. An AAV vector such as that described in Tratschin J D et al., Mol. Cell. Biol. 5:3251-60 (1985), can be used to introduce DNA into cells. A variety of nucleic acids have been introduced into different cell types using AAV vectors (see, e.g., Hermonat P L and Muzyczka N, Proc. Natl. Acad. Sci. USA 81:6466-70 (1984); Tratschin J D et al., Mol. Cell. Biol. 4:2072-81 (1985); Wondisford F E et al., Mol. Endocrinol. 2:32-39 (1988); Tratschin J D et al., J. Virol. 51:611-19 (1984); and Flotte T R et al., J. Biol. Chem. 268:3781-90 (1993)).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection, or reverse transcriptase-polymerase chain reaction (RT-PCR). The gene product can be detected by an appropriate assay, for example by immunological detection of a produced protein, such as with a specific antibody, or by a functional assay to detect a functional activity of the gene product.

1. Prophylactic Methods

In one aspect, the invention provides a method for preventing in a subject, a disease or condition that would benefit from modulation of Ccdc80 activity and/or expression, e.g., obesity, insulin resistance, and/or type 2 diabetes, by administering to the subject a Ccdc80 polypeptide, a Ccdc80 polynucleotide, or an agent that modulates Ccdc80 polypeptide expression or at least one Ccdc80 activity. Subjects at risk for a disease which is caused or contributed to by aberrant Ccdc80 expression or activity can be identified by, for example, any or a combination of diagnostic or prognostic assays as are known to those of ordinary skill in the art. Administration of a prophylactic agent can occur prior to the manifestation of symptoms characteristic of Ccdc80 aberrance, such that a disease or disorder is prevented or, alternatively, delayed in its progression. Depending on the type of Ccdc80 aberrance or condition, for example, a Ccdc80 polypeptide, Ccdc80 polynucleotide, or Ccdc80 agonist agent can be used for treating the subject. The appropriate agent can be determined based on screening assays described herein.

2. Therapeutic Methods

Another aspect of the invention pertains to methods of modulating Ccdc80 expression or activity for therapeutic purposes. Accordingly, in an exemplary embodiment, the modulatory method of the invention involves contacting a cell with a Ccdc80 polypeptide or agent that modulates one or more of the activities of Ccdc80 protein associated with the cell. An agent that modulates Ccdc80 protein activity can be an agent as described herein, such as a nucleic acid or a protein, a naturally-occurring target molecule of a Ccdc80 protein (e.g., a Ccdc80 binding protein), a Ccdc80 agonist, a peptidomimetic of a Ccdc80 agonist, or other small molecule. In one embodiment, the agent stimulates one or more Ccdc80 activities. Examples of such stimulatory agents include active Ccdc80 protein and a nucleic acid molecule encoding Ccdc80 polypeptide that has been introduced into the cell. In another embodiment, the agent inhibits one or more Ccdc80 activities. Examples of such inhibitory agents include, e.g., antisense Ccdc80 nucleic acid molecules, anti-Ccdc80 antibodies, and Ccdc80 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder that would benefit from modulation of a Ccdc80 protein, e.g., obesity, insulin resistance, and/or type 2 diabetes, or which is characterized by aberrant expression or activity of a Ccdc80 protein or nucleic acid molecule. In one embodiment, the method involves administering an agent (e.g., an agent identified by a screening assay described herein), or combination of agents that modulates Ccdc80 expression or activity. In another embodiment, the method involves administering a Ccdc80 protein or nucleic acid molecule as therapy to compensate for reduced or aberrant Ccdc80 expression or activity.

Stimulation of Ccdc80 activity is desirable in situations in which Ccdc80 is abnormally downregulated and/or in which increased Ccdc80 activity is likely to have a beneficial effect. Likewise, inhibition of Ccdc80 activity is desirable in situations in which Ccdc80 is abnormally upregulated and/or in which decreased Ccdc80 activity is likely to have a beneficial effect. Exemplary situations in which Ccdc80 modulation will be desirable are in the treatment of conditions such as obesity, insulin resistance, and/or type 2 diabetes.

Generally, diseases associated with adipogenesis include body weight disorders such as obesity and cachexia, and nonshivering and shivering thermogenesis. Accordingly, in one aspect, Ccdc80 modulators are potentially useful for modulating body weight-related processes, including, for example, treatment of body weight disorders such as obesity and cachexia, and thermogenesis. Depending on the desired result, a Ccdc80 modulator identified to induce adipogenesis is potentially useful for increasing body weight and a Ccdc80 modulator identified to prevent adipogenesis is potentially useful for decreasing body weight.

For obesity, various markers can be used to determine patients that are obese, including a body mass index (BMI) greater than or equal to 30 or greater than or equal to 27 with co-morbid conditions; patients that are overweight include those having a BMI greater than or equal to 25. Co-morbid conditions include cardiovascular (hypertension and atherosclerosis), metabolic (diabetes and hyperlipidemia), liver (biliary disease and gall stones), pulmonary (sleep apnea and respiratory insufficiency) and psychological (lack of self esteem and depression) complications. In one embodiment, successful treatment of obesity is 5-10% or greater reduction in BMI.

Diseases associated with adipogenesis also include type 2 diabetes, insulin resistance, dyslipidemia, hepatic steatosis and the metabolic syndrome. In particular, partial inhibition of adipogenesis has been shown to decrease body weight and improve insulin resistance, plasma lipid profile and hepatic steatosis in mice (Wright W S et al., Diabetes, 56:295-303 (2007); Rosen E D & MacDougald O A, Nat. Rev. Mol. Cell. Biol. 7:885-96 (2006); Millward C A et al., Diabetes 56:161-67 (2007)). Treatments that decrease, but do not completely inhibit, adipogenesis may therefore be beneficial for obesity-associated disorders such as type 2 diabetes, insulin resistance, dyslipidemia, hepatic steatosis and the metabolic syndrome. Some of the beneficial effect of partially blocking adipocyte differentiation may be mediated by altered adipocyte metabolism and/or altered secretion of adipokines (Millward C A et al., supra; Wright W S et al., supra). It is important to note that complete inhibition of adipogenesis is detrimental and results in disorders such as lipodystrophy, insulin resistance and type 2 diabetes in both mice and humans (Reitman M L, Annu. Rev. Nutr. 22:459-82 (2002); Agarwal A K & Garg A, Annu. Rev. Genomics Hum. Genet. 7:175-99 (2006)). Increasing Ccdc80 expression or function may be beneficial under those circumstances.

Additionally, there exists a high correlation between hepatic glucose production, fasting glucose production, and overall metabolic control (as assessed by glycohemoglobin levels) (Galloway et al., Clin. Therap. 12: 460-72 (1990)); thus, control of fasting blood glucose is important for achieving overall normalization of metabolism sufficient to prevent complications of hyperglycemia.

A diabetic subject is a subject, e.g., a human subject, who has been diagnosed as having diabetes (or would be diagnosed as having diabetes) by a skilled medical practitioner or researcher. Exemplary tests utilized in diabetes diagnosis include the fasting plasma glucose (FPG) test and the glucose tolerance test, e.g., the 75-g oral glucose tolerance test (OGTT). Exemplary criteria for the diagnosis of diabetes are set forth in Table 1.

TABLE 1

| Normoglycemia | IFG or IGT¹ | Diabetes² |
|---|---|---|
| FPG < 110 | FPG ≧ 110 and < 126 | FPG ≧ 126 mg/dl |
| mg/dl | mg/dl (IFG) | |
| 2-h PG³ < | 2-h PG³ ≧ 140 and < | 2-h PG³ ≧ 200 mg/dl |
| 140 mg/dl | 200 mg/dl (IGT) | |
| | | Symptoms of diabetes and |
| | | casual plasma glucose |
| | | concentration ≧ 200 mg/dl |

¹Midrange values indicating impaired glucose tolerance (IGT), or impaired fasting glucose (IFG).
²A diagnosis of diabetes must be confirmed, on a subsequent day, by measurement of FPG, 2-h PG (plasma glucose), or random plasma glucose (if symptoms are present). Fasting is defined as no caloric intake for at least 8 h.
³This test requires the use of a glucose load containing the equivalent of 75 g anhydrous glucose dissolved in water. 2-h PG, 2-h postload glucose.

An insulin resistant subject is a subject, e.g., a human subject, who has been diagnosed as being insulin resistant (or would be diagnosed as being insulin resistant) by a skilled medical practitioner or researcher. An insulin resistant subject can be identified, for example, by determining fasting glucose and/or insulin levels in said subject. In a preferred embodiment, an insulin resistant subject has a fasting glucose level of less than 110 mg/dL and has a fasting insulin level of greater that 30 mU/L.

With respect to cosmetic treatment of obesity, a subject having excess body weight in the form of fat can be identified visually and/or by having a BMI greater than or equal to 25. Such subjects would be considered to be overweight and in need of weight control for cosmetic treatment. An agent that modulates the expression or activity of the Ccdc80 gene or Ccdc80 protein may be used as a cosmetic product for reducing excess body weight in the form of fat in these subjects. The subject in need of cosmetic treatment of obesity would be administered a composition including the agent that modulates the expression or activity of the Ccdc80 gene or Ccdc80 protein.

### B. Combination Treatments

Ccdc80 modulators may also be used in conjunction with other therapeutic agent(s), preferably those commonly used for treating the particular disease associated with adipogenesis according to the present methods. Suitable therapeutic agents for combination therapies related to type 2 diabetes include, for example, insulins. Insulins useful with the methods and combinations of this invention include rapid acting insulins, intermediate acting insulins, long acting insulins and combinations of intermediate and rapid acting insulins. Insulin therapy replaces insulin that is not being produced by the body. The combination of a rapid- or short-acting and intermediate- or long-acting insulin helps keep blood sugar levels within normal or closer to normal levels. The use of these agents is described in further detail in Published U.S. Patent Application No. 2002/0187980, relevant portions thereof are herein incorporated by reference.

Also useful in type 2 diabetes combination therapy with Ccdc80 modulators are sulfonylurea agents. Sulfonylurea agents increase the amount of insulin produced by the pancreas. They also increase the effectiveness of insulin throughout the body by increasing functionality of insulin receptors and stimulating the production of more insulin receptors. These agents also reduce insulin resistance and may reduce the amount of sugar made by the liver. Sulfonylurea agents useful with the methods and compositions of this invention include glipizide, glyburide (glibenclamide), chlorpropamide, tolbutamide, tolazamide and glimepriride, or the pharmaceutically acceptable salt forms thereof. The use of these agents are described in further detail in Published U.S. Patent Application No. 2003/008869, relevant portions of which are herein incorporated by reference.

Another therapeutic agent useful in combination with Ccdc80 modulators in type 2 diabetes treatment is a biguanide agent. Biguanide agents lower blood sugar by decreasing the amount of sugar produced by the liver in gluconeogenesis. They also increase the amount of sugar absorbed by muscle cells and decrease insulin resistance. These agents may lower triglyceride levels in the blood and reduce certain abnormal clotting factors and markers of inflammation that can lead to atherosclerosis. Biguanide agents useful with the methods and compositions of this invention include mefformin and its pharmaceutically acceptable salt forms. The use of these agents is described in further detail in Published U.S. Patent Pub. No. 2003/0018028, relevant portions thereof are herein incorporated by reference.

Thiazolidinedione agents can also be used in combination with Ccdc80 modulators in the treatment of type 2 diabetes. Thiazolidinedione agents improve the way cells in the body respond to insulin by lowering insulin resistance. They also may help in the treatment of high cholesterol by reducing triglycerides and increasing high-density lipoproteins (HDL) in the blood. Thiazolidinedione agents useful with the methods and compositions of this invention are the non-limiting group of pioglitazone or rosiglitazone, or a pharmaceutically acceptable salt form of these agents. The use of these agents is described in further detail in Published U.S. Patent Application No. 2002/0198203, relevant portions thereof are herein incorporated by reference.

Also useful in type 2 diabetes combination therapies with Ccdc80 modulators are alpha-glucosidase inhibitors. Alpha-glucosidase inhibitors delay the digestion of carbohydrates in the body and slow the rate at which the intestines absorb glucose from food. This decreases the amount of sugar that passes into your blood after a meal and prevents periods of hyperglycemia. Alpha-glucosidase inhibitors which may be used with the methods and compositions of the invention described herein are miglitol or acarbose, or a pharmaceutically acceptable salt form of one or more of these compounds. The use of these agents is described in further detail in Published U.S. Patent Application No. 2003/0013709, relevant portions thereof are herein incorporated by reference.

Another therapeutic agent useful in combination with Ccdc80 modulators in type 2 diabetes treatment is an antilipemic agent. Antilipemic agents, also known as antihyperlipidemic agents, which may be utilized with the methods and compositions of the invention described herein are bile acid sequestrants, fibric acid derivatives, HMG-CoA reductase inhibitors and nicotinic acid compounds. Antilipemic agents reduce the amount of cholesterol and fats in the blood through a number of mechanisms. For example, bile acid sequestrants bind to bile acids in the intestine and prevent them from being reabsorbed into the blood. The liver then produces more bile to replace the bile which has been lost. Since the body needs cholesterol to make bile, the liver uses up the cholesterol in the blood, reducing the amount of LDL cholesterol circulating in the blood. The use of these agents is described in further detail in Published U.S. Patent Application No. 2002/0198202, relevant portions thereof are herein incorporated by reference.

Also useful in type 2 diabetes combination therapy with Ccdc80 modulators are angiotensin converting enzyme (ACE) inhibitors. ACE inhibitors dilate blood vessels to improve the amount of blood the heart pumps and lower blood pressure. ACE inhibitors also increase blood flow, which helps to decrease the amount of work the heart has to do. ACE inhibitors useful in the methods and compositions disclosed herein include quinapril, ramipril, verapamil, captopril, diltiazem, clonidine, hydrochlorthiazide, benazepril, prazosin, fosinopril, lisinopril, atenolol, enalapril, perindropril, perindropril tert-butylamine, trandolapril and moexipril, or a pharmaceutically acceptable salt form of one or more of these compounds. The use of these agents is described in further detail in Published U.S. Patent Application No. 2003/0055058, relevant portions thereof are herein incorporated by reference.

In relation to secondary diabetic effects, aldose reductase inhibitors prevent eye and nerve damage in people with diabetes. Aldose reductase is an enzyme that is normally present in the eye and triggers the metabolism of glucose into sorbitol, which can damage the eye. Aldose reductase inhibitors slow this process. Among the aldose reductase inhibitors useful in combination with Ccdc80 modulators are minalrestat Tolrestat, Sorbinil, Methosorbinil, Zopolrestat, Epalrestat, Zenarestat Imirestat, and Ponalrestat or the pharmaceutically acceptable salt forms thereof. The use of these agents is described in further detail in Published U.S. Patent Application No. 2002/0198201, relevant portions thereof are herein incorporated by reference.

Suitable therapeutic agents for combination therapies related to obesity include, for example, central nervous system (CNS) stimulants (e.g., phentermines (e.g., those sold under the tradenames Ionamin® and Adipex-P®). The phentermines are members of a class of drugs known as the sympathomimetics for their ability to mimic stimulation of the central nervous system. The phentermines act on the hypothalamus, an appetite control center of the brain. Phentermine monotherapy can increase weight loss when used in combination with diet and exercise, as compared to diet and exercise alone. The use of these agents is described in further detail in U.S. Pat. No. 5,019,594, relevant portions thereof are herein incorporated by reference

Also useful in obesity combination therapy with Ccdc80 modulators are re-uptake inhibitors. Re-uptake inhibitors suppress appetite by inhibiting the re-uptake of the neurotransmitters serotonin, norepinephrine, and dopamine. Re-uptake inhibitors useful in combination with Ccdc80 modulators include 5HT-2C inhibitors (e.g., Meridia® (sibutramine), Lorcaserin (APD-356)). The use of these agents is described in further detail in U.S. Pat. No. 4,929,629, relevant portions thereof are herein incorporated by reference.

Another therapeutic agent useful in combination with Ccdc80 modulators in obesity treatment is a CB-1 antagonists. CB-1 antagonists act by blocking endogenous cannabinoid binding to neuronal CB-1 receptors. By blocking cannibinoid receptors, CB-1 antagonists reduce appetite in a subject. Useful CB-1 antagonists include rimonabant (Acomplia®) and CP-945598. The use of these agents is described in further detail in U.S. Pat. No. 5,624,941, relevant portions thereof are herein incorporated by reference.

Also useful in obesity combination therapy with Ccdc80 modulators are GLP-1 agonists or mimetics. GLP-1 agonists normalize hyperglycemia through glucose-dependent, insulin-dependent and insulin-independent mechanisms. GLP-1 agonists are useful as primary agents for the treatment of type 2 diabetes and as adjunctive agents for the treatment of type 1 diabetes. Useful GLP-1 agonists and mimetics include exenatide (Byetta®). The use of these agents is described in further detail in U.S. Pat. No. 5,424,286, relevant portions thereof are herein incorporated by reference.

The order of administration of a Ccdc80 modulator and an additional therapeutic agent(s) can vary. For example, in some embodiments, a Ccdc80 modulator is administered concurrently with the additional therapeutic agent(s). Alternatively, a Ccdc80 modulator can be administered separately and prior to the additional therapeutic agent(s). In another embodiment, the additional therapeutic agent(s) can be administered separately and prior to a Ccdc80 modulator. In many embodiments, these administration regimens will be continued for days, months, or years.

### C. Screening Assays:

The invention provides a method (also referred to herein as a “screening assay”) for identifying modulators, that is, candidate or test compounds or agents (e.g., peptides, peptidomimetics, small molecules, or other drugs) which bind to Ccdc80 proteins, or have a stimulatory or inhibitory effect on, for example, Ccdc80 expression or Ccdc80 activity.

The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer, or small molecule libraries of compounds (Lam K S, Anticancer Drug Des. 12:145-67 (1997)).

Examples of methods for the synthesis of molecular libraries can be found in the art, for example in: DeWitt S H et al., Proc. Natl. Acad. Sci. USA 90:6909-13 (1993); Erb E et al., Proc. Natl. Acad. Sci. USA 91:11422-26 (1994); Zuckermann R N et al., J. Med. Chem. 37:2678-85 (1994); Cho C Y et al., Science 261:1303-05 (1993); Carrell T et al., Angew. Chem. Int. Ed. Engl. 33:2059-61 (1994); Carrell T et al., Angew. Chem. Int. Ed. Engl. 33:2061-64 (1994); and Gallop M A et al., J. Med. Chem. 37:1233-51 (1994).

Libraries of compounds may be presented, for example, in solution (e.g., Houghten R A et al., Biotechniques 13:412-21 (1992)), on beads (Lam K S et al., Nature 354:82-84 (1991)), chips (Fodor S P A et al., Nature 364:555-56 (1993)), bacteria (U.S. Pat. No. 5,223,409), spores (U.S. Pat. No. 5,223,409), plasmids (Cull M G et al., Proc. Natl. Acad. Sci. USA 89:1865-69 (1992)), or on phage (Scott J K and Smith G P, Science 249:386-90 (1990); Devlin J J et al., Science 249:404-06 (1990); Cwirla S E et al., Proc. Natl. Acad. Sci. 87:6378-82 (1990); Felici F et al., J. Mol. Biol. 222:301-10 (1991); U.S. Pat. No. 5,223,409).

In many drug screening programs which test libraries of modulating agents and natural extracts, high throughput assays are desirable in order to maximize the number of modulating agents surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test modulating agent. Moreover, the effects of cellular toxicity and/or bioavailability of the test modulating agent can be generally ignored in the in vitro system, the assay instead being focused primarily on the effect of the drug on the molecular target as may be manifest in an alteration of binding affinity with upstream or downstream elements.

Assays can be used to screen for modulating agents, including Ccdc80 homologs, which are either agonists or antagonists of the normal cellular function of the subject Ccdc80 polypeptides. For example, the invention provides a method in which an indicator composition is provided which has a Ccdc80 protein having a Ccdc80 activity. The indicator composition can be contacted with a test compound. The effect of the test compound on Ccdc80 activity, as measured by a change in the indicator composition, can then be determined to thereby identify a compound that modulates the activity of a Ccdc80 protein. A statistically significant change, such as a decrease or increase, in the level of Ccdc80 activity in the presence of the test compound (relative to what is detected in the absence of the test compound) is indicative of the test compound being a Ccdc80 modulating agent. The indicator composition can be, for example, a cell or a cell extract.

The efficacy of the modulating agent can be assessed by generating dose response curves from data obtained using various concentrations of the test modulating agent. Moreover, a control assay can also be performed to provide a baseline for comparison. In the control assay, isolated and purified Ccdc80 protein is added to a composition containing the Ccdc80-binding element, and the formation of a complex is quantitated in the absence of the test modulating agent.

In yet another embodiment, an assay of the present invention is a cell-free assay in which a Ccdc80 protein or biologically active portion thereof is contacted with a test compound and the ability of the test compound to bind to the Ccdc80 protein or biologically active portion thereof is determined. Binding of the test compound to the Ccdc80 protein can be determined either directly or indirectly as described above. In a preferred embodiment, the assay includes contacting the Ccdc80 protein or biologically active portion thereof with a known compound which binds Ccdc80 to form an assay mixture, contacting the assay mixture with a test compound, and determining the ability of the test compound to interact with a Ccdc80 protein, wherein determining the ability of the test compound to interact with a Ccdc80 protein comprises determining the ability of the test compound to preferentially bind to Ccdc80 polypeptide or a biologically active portion thereof as compared to the known compound.

In another embodiment, the assay is a cell-free assay in which a Ccdc80 protein or biologically active portion thereof is contacted with a test compound and the ability of the test compound to modulate the activity of the Ccdc80 protein or biologically active portion thereof is determined. The Ccdc80 protein can be provided as a lysate of cells that express Ccdc80, as a purified or semipurified polypeptide, or as a recombinantly expressed polypeptide. In one embodiment, a cell-free assay system further comprises a cell extract or isolated components of a cell, such as mitochondria. Such cellular components can be isolated using techniques which are known in the art. Preferably, a cell free assay system further comprises at least one target molecule with which Ccdc80 interacts, and the ability of the test compound to modulate the interaction of the Ccdc80 with the target molecule(s) is monitored to thereby identify the test compound as a modulator of Ccdc80. Determining the ability of the test compound to modulate the activity of a Ccdc80 protein can be accomplished, for example, by determining the ability of the Ccdc80 protein to bind to a Ccdc80 target molecule by one of the methods described herein for determining direct binding. Determining the ability of the Ccdc80 protein to bind to a Ccdc80 target molecule can also be accomplished using a technology such as real-time Biomolecular Interaction Analysis (BIA) (see, e.g., Sjolander S and Urbaniczky C, Anal. Chem. 63:2338-45 (1991) and Szabo A et al., Curr. Opin. Struct. Biol. 5:699-705 (1995)).

In yet another embodiment, the cell-free assay involves contacting a Ccdc80 protein or biologically active portion thereof with a known compound which binds the Ccdc80 protein to form an assay mixture, contacting the assay mixture with a test compound, and determining the ability of the test compound to interact with the Ccdc80 protein, wherein determining the ability of the test compound to interact with the Ccdc80 protein comprises determining the ability of the Ccdc80 protein to preferentially bind to or modulate the activity of a Ccdc80 target molecule.

The cell-free assays of the present invention are amenable to use of both soluble and/or membrane-bound forms of proteins (e.g., Ccdc80 proteins or receptors having intracellular domains to which Ccdc80 binds). In the case of cell-free assays in which a membrane-bound form a protein is used, it may be desirable to utilize a solubilizing agent such that the membrane-bound form of the protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Determining the ability of the Ccdc80 protein to bind to or interact with a ligand of a Ccdc80 molecule can be accomplished, for example, by direct binding. In a direct binding assay, the Ccdc80 protein could be coupled with a radioisotope or enzymatic label such that binding of the Ccdc80 protein to a Ccdc80 target molecule can be determined by detecting the labeled Ccdc80 protein in a complex. For example, Ccdc80 molecules, for example, Ccdc80 proteins, can be labeled with, for example, ¹²⁵I, ³⁵S, ¹⁴C, or ³H, either directly or indirectly, and the radioisotope detected by direct counting of radioemmission or by scintillation counting. Alternatively, Ccdc80 molecules can be enzymatically labeled with, for example, horseradish peroxidase, alkaline phosphatase, or luciferase, and the enzymatic label detected by determination of conversion of an appropriate substrate to product.

Typically, it will be desirable to immobilize Ccdc80 or their respective binding proteins to facilitate separation of complexes from uncomplexed forms of one or both of the proteins, as well as to accommodate automation of the assay. Binding of Ccdc80 to an upstream or downstream binding element, in the presence and absence of a candidate agent, can be accomplished in any vessel suitable for containing the reactants. Examples include microtiter plates, test tubes, and micro-centrifuge tubes. In one embodiment, a fusion protein can be provided which adds a domain that allows the protein to be bound to a matrix. For example, glutathione-S-transferase/Ccdc80 (GST/Ccdc80) fusion proteins can be adsorbed onto glutathione sepharose beads (Sigma Chemical, St. Louis, Mo.) or glutathione derivatized microtiter plates, which are then combined with the cell lysates and the test modulating agent, and the mixture incubated under conditions conducive to complex formation, for example, at physiological conditions for salt and pH, though slightly more stringent conditions may be desired. Following incubation, the beads are washed to remove any unbound label, and the matrix immobilized and radiolabel determined directly (e.g., beads placed in scintillant), or in the supernatant after the complexes are subsequently dissociated. Alternatively, the complexes can be dissociated from the matrix, separated by SDS-PAGE, and the level of Ccdc80-binding protein found in the bead fraction quantitated from the gel using standard electrophoretic techniques.

Other techniques for immobilizing proteins on matrices are also available for use in the subject assay. For instance, Ccdc80 or a cognate binding protein thereof can be immobilized utilizing conjugation of biotin and streptavidin. Biotinylated Ccdc80 molecules can be prepared from biotin-NHS(N-hydroxy-succinimide) using techniques well known in the art (e.g., biotinylation kit, Pierce Biotechnology, Rockford, Ill.), and immobilized in the wells of streptavidin-coated 96 well plates (Pierce Biotechnology). Alternatively, antibodies reactive with Ccdc80 but which do not interfere with binding of upstream or downstream elements can be derivatized to the wells of the plate, and Ccdc80 trapped in the wells by antibody conjugation. As above, preparations of a Ccdc80-binding protein (Ccdc80-8P) and a test modulating agent are incubated in the Ccdc80-presenting wells of the plate, and the amount of complex trapped in the well can be quantitated. Exemplary methods for detecting such complexes, in addition to those described above for the GST-immobilized complexes, include immunodetection of complexes using antibodies reactive with the Ccdc80 binding element, or which are reactive with Ccdc80 protein and compete with the binding element, as well as enzyme-linked assays which rely on detecting an enzymatic activity associated with the binding element, either intrinsic or extrinsic activity. In the instance of the latter, the enzyme can be chemically conjugated or provided as a fusion protein with the Ccdc80 binding protein. To illustrate, the Ccdc80 binding protein can be chemically cross-linked or genetically fused with horseradish peroxidase, and the amount of protein trapped in the complex can be assessed with a chromogenic substrate of the enzyme, for example, 3,3′-diamino-benzadine terahydrochloride or 4-chloro-1-napthol. Likewise, a fusion protein comprising the protein and glutathione-S-transferase can be provided, and complex formation quantitated by detecting the GST activity using 1-chloro-2,4-dinitrobenzene (Habig W H et al., J. Biol. Chem. 249:7130-39 (1974)).

For processes which rely on immunodetection for quantitating one of the proteins trapped in the complex, antibodies against the protein, such as anti-CDC80 antibodies, can be used. Alternatively, the protein to be detected in the complex can be “epitope tagged” in the form of a fusion protein which includes, in addition to the Ccdc80 sequence, a second protein for which antibodies are readily available (e.g., from commercial sources). For instance, the GST fusion proteins described above can also be used for quantification of binding using antibodies against the GST moiety. Other useful epitope tags include myc-epitopes (see, e.g., Ellison M J and Hochstrasser M, J. Biol. Chem. 266:21150-57 (1991)) which includes a 10-residue sequence from c-myc, as well as the pFLAG® system (SigmaAldrich, St. Louis, Mo.) or the pEZZ-protein A system (GE Healthcare, Piscataway, N.J.).

It is also within the scope of this invention to determine the ability of a compound to modulate the interaction between Ccdc80 and its target molecules without the labeling of any of the interactants. For example, a microphysiometer can be used to detect the interaction of Ccdc80 with its target molecules without the labeling of Ccdc80 or the target molecules (see, e.g., McConnell H M et al., Science 257:1906-12 (1992)). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

In addition to cell-free assays, the readily available source of Ccdc80 proteins provided by the present invention also facilitates the generation of cell-based assays for identifying small molecule agonists/antagonists and the like. For example, cells can be caused to express or overexpress a recombinant Ccdc80 protein in the presence and absence of a test modulating agent of interest, with the assay scoring for modulation in Ccdc80 responses by the target cell mediated by the test agent. For example, as with the cell-free assays, modulating agents which produce a statistically significant change in Ccdc80-dependent responses (either an increase or decrease) can be identified.

Recombinant expression vectors that can be used for expression of Ccdc80 are known in the art (see discussions above). In one embodiment, within the expression vector the Ccdc80-coding sequences are operably linked to regulatory sequences that allow for constitutive or inducible expression of Ccdc80 in the indicator cell(s). Use of a recombinant expression vector that allows for constitutive or inducible expression of Ccdc80 in a cell is preferred for identification of compounds that enhance or inhibit the activity of Ccdc80. In an alternate embodiment, within the expression vector, the Ccdc80 coding sequences are operably linked to regulatory sequences of the endogenous Ccdc80 gene (i.e., the promoter regulatory region derived from the endogenous gene). Use of a recombinant expression vector in which Ccdc80 expression is controlled by the endogenous regulatory sequences is preferred for identification of compounds that enhance or inhibit the transcriptional expression of Ccdc80. In one embodiment, an assay is a cell-based assay comprising contacting a cell expressing a Ccdc80 target molecule (e.g., a Ccdc80 intracellular interacting molecule) with a test compound and determining the ability of the test compound to modulate (e.g., stimulate or inhibit) the activity of the Ccdc80 target molecule. Determining the ability of the test compound to modulate the activity of a Ccdc80 target molecule can be accomplished, for example, by determining the ability of the Ccdc80 protein to bind to or interact with the Ccdc80 target molecule or its ligand.

In an illustrative embodiment, the expression or activity of Ccdc80 is modulated in cells and the effects of modulating agents of interest on the readout of interest (such as, e.g., preadipocyte proliferation and/or lipid accumulation) are measured and/or observed.

In another embodiment, determining the ability of a Ccdc80 modulator to bind to or interact with a target molecule can be accomplished by measuring a read out of the activity of Ccdc80 or of the target molecule. For example, the activity of Ccdc80 or a target molecule can be determined by detecting induction of a cellular second messenger of the target, detecting catalytic/enzymatic activity of the target an appropriate substrate, detecting the induction of a reporter gene (comprising a target-responsive regulatory element operably linked to a nucleic acid encoding a detectable marker, e.g., chloramphenicol acetyl transferase), or detecting a target-regulated cellular response, for example, preadipocyte proliferation and/or lipid accumulation.

### VI. ADMINISTRATION OF Ccdc80 MODULATORS

Ccdc80 modulators are administered to subjects in a biologically compatible form suitable for pharmaceutical administration in vivo to either enhance or suppress Ccdc80 activity. By “biologically compatible form suitable for administration in vivo” is meant a form of the Ccdc80 modulator to be administered in which any toxic effects are outweighed by the therapeutic effects of the modulator. The term subject is intended to include living organisms in which an immune response can be elicited, for example, mammals. Administration of Ccdc80 modulators as described herein can be in any pharmacological form including a therapeutically active amount of an agent alone or in combination with a pharmaceutically acceptable carrier.

Administration of a therapeutically active amount of the Ccdc80 modulators of the present invention is defined as an amount effective, at dosages and for periods of time necessary, to achieve the desired result. For example, a therapeutically active amount of a Ccdc80 modulator may vary according to factors such as the disease state, age, sex, and weight of the individual, and the ability of peptide to elicit a desired response in the individual. Dosage regima may be adjusted to provide the optimum therapeutic response. For example, several divided doses may be administered daily, or the dose may be proportionally reduced as indicated by the exigencies of the therapeutic situation.

The therapeutic or pharmaceutical compositions of the present invention can be administered by any suitable route known in the art including, for example, intravenous, subcutaneous, intramuscular, transdermal, intrathecal, or intracerebral or administration to cells in ex vivo treatment protocols. Administration can be either rapid as by injection or over a period of time as by slow infusion or administration of slow release formulation.

Ccdc80 modulators can also be linked or conjugated with agents that provide desirable pharmaceutical or pharmacodynamic properties. For example, Ccdc80 modulators can be coupled to any substance known in the art to promote penetration or transport across the blood-brain barrier such as an antibody to the transferrin receptor, and administered by intravenous injection (see, e.g., Friden P M et al., Science 259:373-77 (1993)). Furthermore, Ccdc80 modulators can be stably linked to a polymer such as polyethylene glycol to obtain desirable properties of solubility, stability, half-life, and other pharmaceutically advantageous properties (see, e.g., Davis et al., Enzyme Eng. 4:169-73 (1978); Burnham N L, Am. J. Hosp. Pharm. 51:210-18 (1994)).

Furthermore, Ccdc80 modulators can be in a composition which aids in delivery into the cytosol of a cell. For example, a Ccdc80 modulator may be conjugated with a carrier moiety such as a liposome that is capable of delivering the peptide into the cytosol of a cell. Such methods are well known in the art (see, e.g., Amselem S et al., Chem. Phys. Lipids 64:219-37 (1993)). Alternatively, a Ccdc80 modulator can be modified to include specific transit peptides or fused to such transit peptides which are capable of delivering the Ccdc80 modulator into a cell. In addition, the modulator can be delivered directly into a cell by microinjection.

The Ccdc80 modulators are usually employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. Ccdc80 modulators can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion by continuous or periodic infusion.

Dose administration can be repeated depending upon the pharmacokinetic parameters of the dosage formulation and the route of administration used.

It is also provided that certain formulations containing the Ccdc80 modulators are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents, or flavoring agents. The compositions may be formulated so as to provide rapid, sustained, or delayed release of the active ingredients after administration to the patient by employing procedures well known in the art. The formulations can also contain substances that diminish proteolytic degradation and/or substances which promote absorption such as, for example, surface active agents.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the Ccdc80 modulator and the particular therapeutic effect to be achieved and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

Toxicity and therapeutic efficacy of such Ccdc80 modulators can be determined by standard pharmaceutical procedures in cell cultures or experimental animals, for example, for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Ccdc80 modulators which exhibit large therapeutic indices are preferred. While Ccdc80 modulators that exhibit toxic side effects may be used, care should be taken to design a delivery system that targets such modulators to the site of affected tissue in order to minimize potential damage to uninfected cells and, thereby, reduce side effects.

The data obtained from the cell culture assays and animal studies can be used in formulating a range of dosage for use in humans. The dosage of such Ccdc80 modulators lies preferably within a range of circulating concentrations that include the ED₅₀ with little or no toxicity. The dosage may vary within this range depending upon the dosage form employed and the route of administration utilized. For any Ccdc80 modulator used in the method of the invention, the therapeutically effective dose can be estimated initially from cell culture assays. A dose may be formulated in animal models to achieve a circulating plasma concentration range that includes the IC₅₀ (i.e., the concentration of the Ccdc80 modulator that achieves a half-maximal inhibition of symptoms) as determined in cell culture. Such information can be used to more accurately determine useful doses in humans. Levels in plasma may be measured, for example, by high performance liquid chromatography.

In one embodiment of this invention, a Ccdc80 polypeptide may be therapeutically administered by implanting into patients vectors or cells capable of producing a biologically-active form of Ccdc80 or a precursor of Ccdc80, that is, a molecule that can be readily converted to a biological-active form of Ccdc80 by the body.

In one approach, cells that secrete Ccdc80 may be encapsulated into semipermeable membranes for implantation into a patient. The cells can be cells that normally express Ccdc80 or a precursor thereof or the cells can be transformed to express Ccdc80 or a biologically active fragment thereof or a precursor thereof. It is preferred that the cell be of human origin. However, the formulations and methods herein can be used for veterinary as well as human applications and the term “patient” or “subject” as used herein is intended to include human and veterinary patients.

Monitoring the influence of Ccdc80 modulators on the expression or activity of a Ccdc80 protein can be applied not only in basic drug screening, but also in clinical trials. For example, the effectiveness of a Ccdc80 modulator determined by a screening assay as described herein to increase Ccdc80 gene expression, protein levels, or upregulate Ccdc80 activity, can be monitored in clinical trials of subjects exhibiting decreased Ccdc80 gene expression, protein levels, or downregulated Ccdc80 activity. Alternatively, the effectiveness of an agent determined by a screening assay to decrease Ccdc80 gene expression, protein levels, or downregulate Ccdc80 activity, can be monitored in clinical trials of subjects exhibiting increased Ccdc80 gene expression, protein levels, or upregulated Ccdc80 activity. In such clinical trials, the expression or activity of a Ccdc80 gene, and preferably other genes that have been implicated in a disorder can be used as a “read out” or markers of the phenotype of a particular cell.

For example, and not by way of limitation, genes, including Ccdc80, that are modulated in cells by treatment with a Ccdc80 modulator (e.g., compound, drug, or small molecule) that modulates Ccdc80 activity (e.g., identified in a screening assay as described herein) can be identified. Thus, to study the effect of agents on a Ccdc80 associated disorder, for example, in a clinical trial, cells can be isolated and RNA prepared and analyzed for the levels of expression of Ccdc80 and other genes implicated in the Ccdc80-associated disorder, respectively. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of Ccdc80 or other genes. In this way, the gene expression pattern can serve as a marker, indicative of the physiological response of the cells to the agent. Accordingly, this response state may be determined before, and at various points during, treatment of the individual with the Ccdc80 modulator.

The present invention also provides a method for monitoring the effectiveness of treatment of a subject with a Ccdc80 modulator (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the Ccdc80 modulator; (ii) detecting the level of expression of a Ccdc80 protein, mRNA, or genomic DNA in the pre-administration sample; (iii) obtaining one or more post-administration samples from the subject; (iv) detecting the level of expression or activity of the Ccdc80 protein, mRNA, or genomic DNA in the post-administration samples; (v) comparing the level of expression or activity of the Ccdc80 protein, mRNA, or genomic DNA in the pre-administration sample with the Ccdc80 protein, mRNA, or genomic DNA in the post administration sample or samples; and (vi) altering the administration of the Ccdc80 modulator to the subject accordingly. For example, increased administration of the Ccdc80 modulator may be desirable to increase the expression or activity of Ccdc80 to higher levels than detected, that is, to increase the effectiveness of the agent. Alternatively, decreased administration of the agent may be desirable to decrease expression or activity of Ccdc80 to lower levels than detected, that is, to decrease the effectiveness of the Ccdc80 modulator. According to such an embodiment, Ccdc80 expression or activity may be used as an indicator of the effectiveness of a Ccdc80 modulator, even in the absence of an observable phenotypic response.

In a preferred embodiment, the ability of a Ccdc80 modulator to alter preadipocyte proliferation and/or lipid accumulation in a subject that would benefit from modulation of the expression and/or activity of Ccdc80 can be measured by detecting an improvement in the condition of the patient after the administration of the Ccdc80 modulator. Such improvement can be readily measured by one of ordinary skill in the art using indicators appropriate for the specific condition of the patient. Monitoring the response of the patient by measuring changes in the condition of the patient is preferred in situations were the collection of biopsy materials would pose an increased risk and/or detriment to the patient.

Furthermore, in the treatment of disease conditions, compositions containing Ccdc80 can be administered exogenously and it would likely be desirable to achieve certain target levels of Ccdc80 polypeptide in sera, in any desired tissue compartment, or in the affected tissue. It would, therefore, be advantageous to be able to monitor the levels of Ccdc80 polypeptide in a patient or in a biological sample including a tissue biopsy sample obtained from a patient and, in some cases, also monitoring the levels of native Ccdc80. Accordingly, the present invention also provides methods for detecting the presence of Ccdc80 in a sample from a patient.

### EXAMPLES

The present invention is further defined in the following Examples. It should be understood that these Examples, while indicating preferred embodiments of the invention, are given by way of illustration only. From the above discussion and these Examples, one skilled in the art can ascertain the preferred features of this invention, and without departing from the spirit and scope thereof, can make various changes and modification of the invention to adapt it to various uses and conditions.

### Example 1

### Experimental Procedures

### 1. Gene Expression Profiling and Quantitative RT-PCR

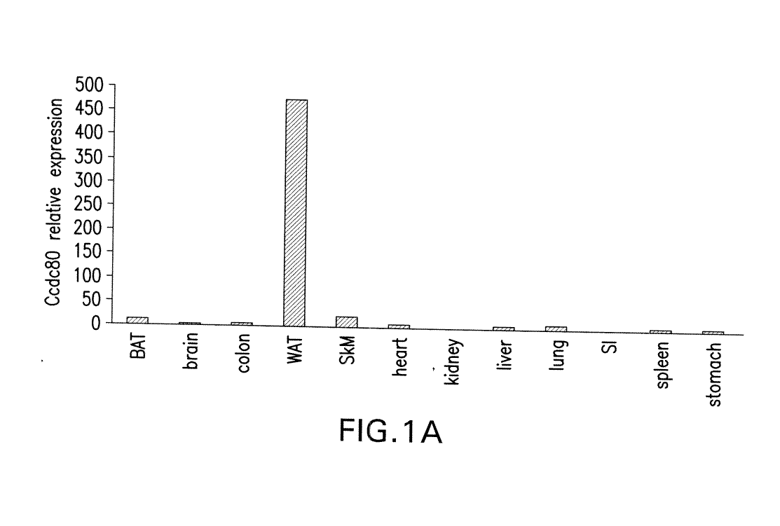

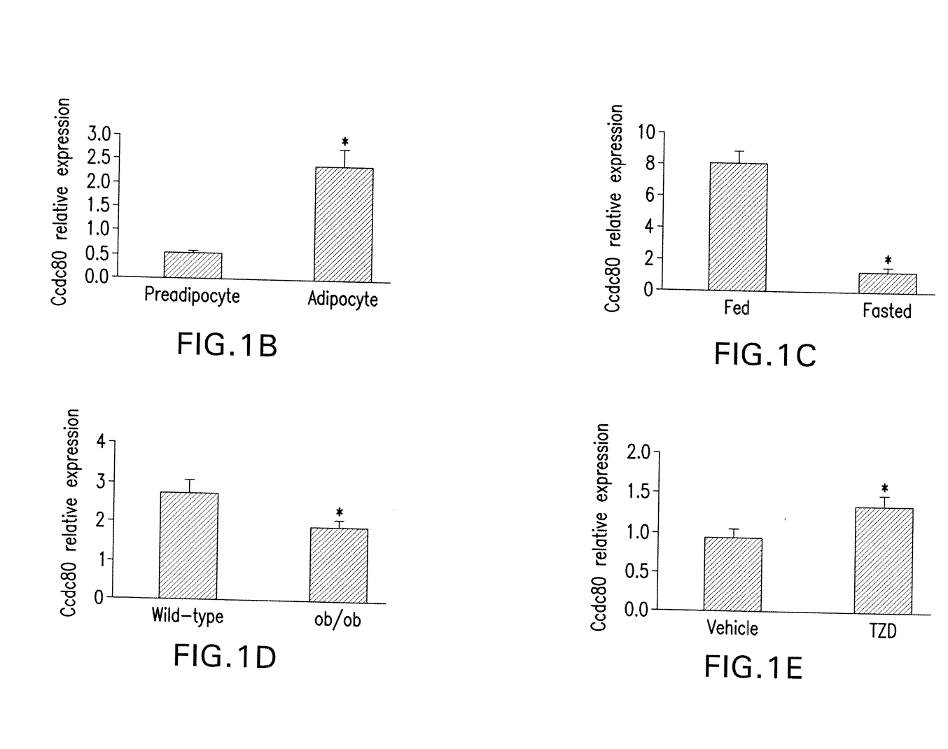

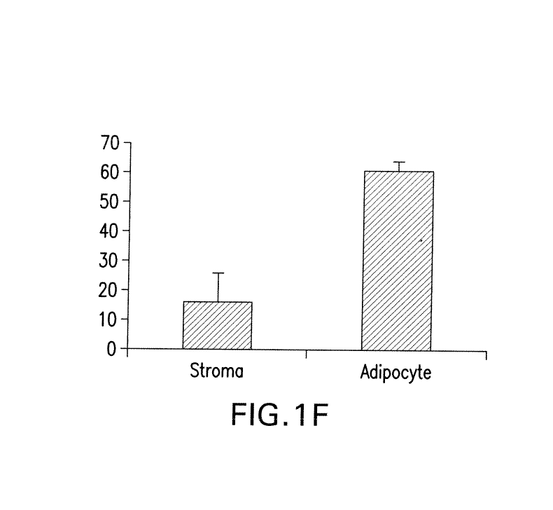

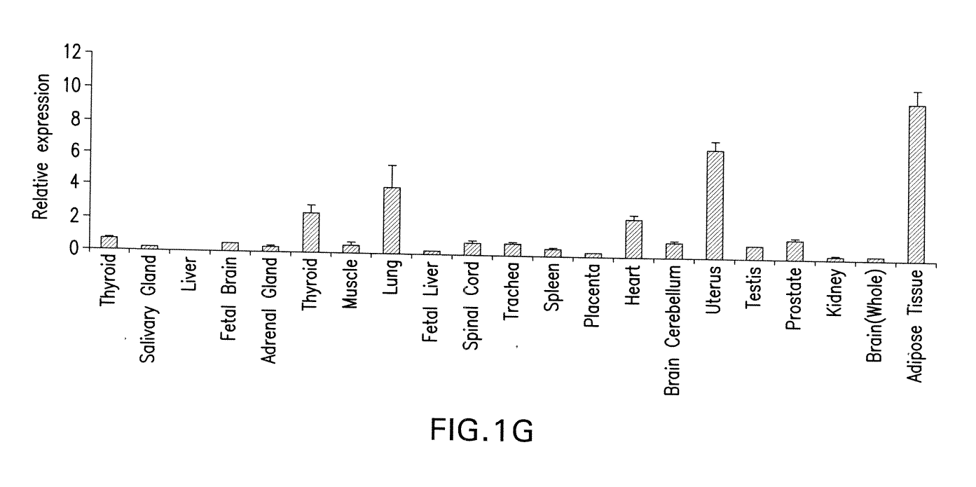

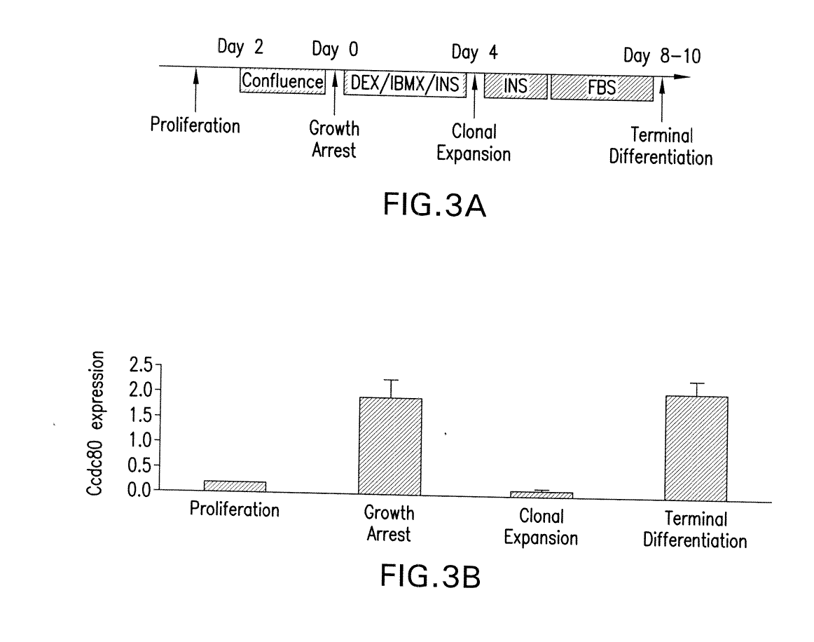

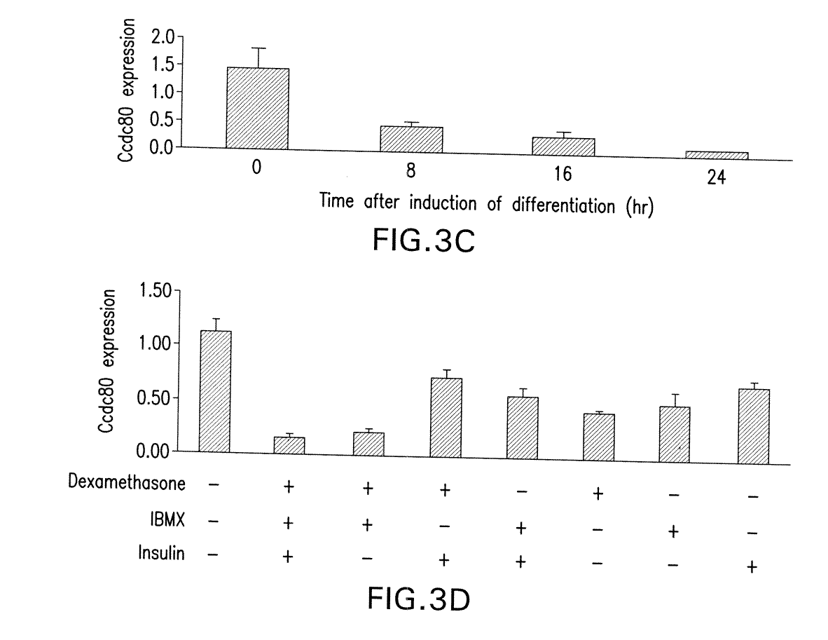

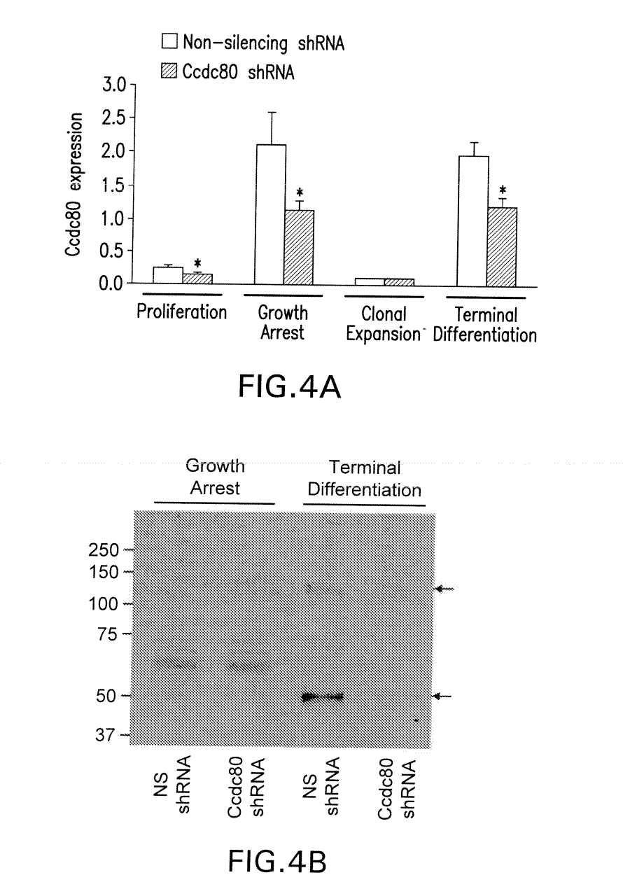

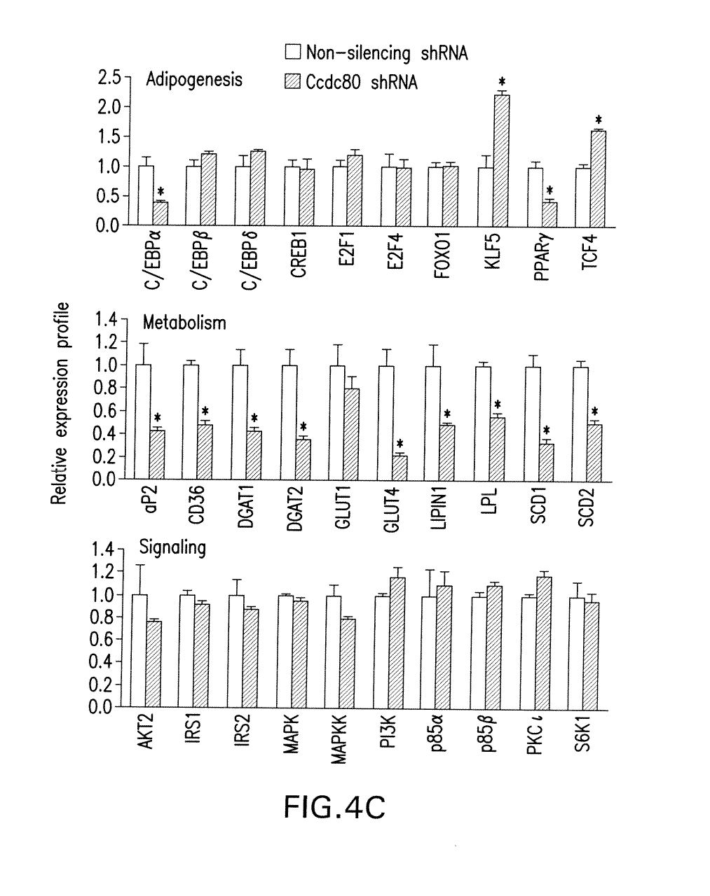

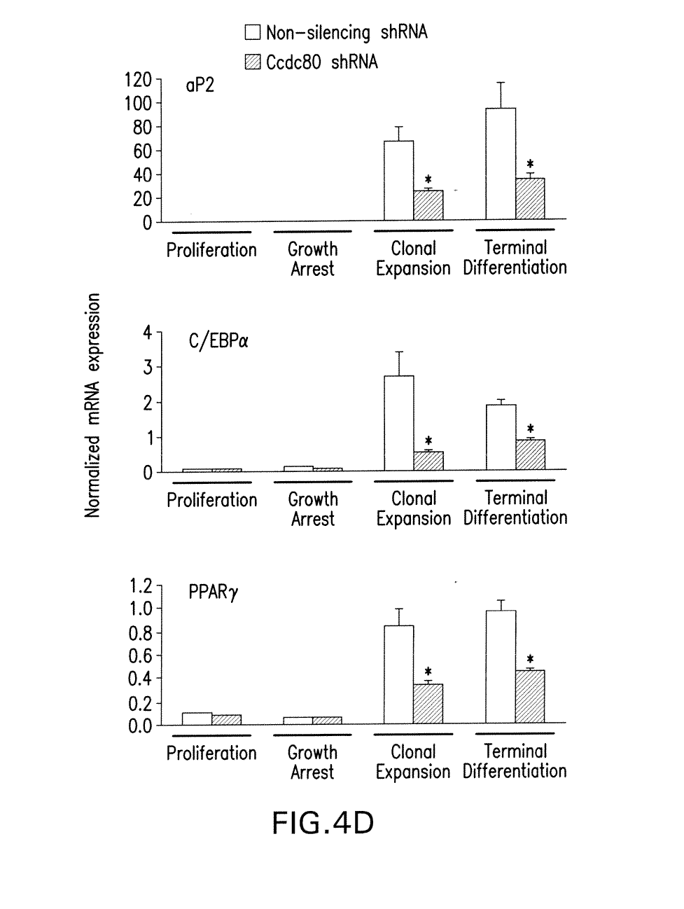

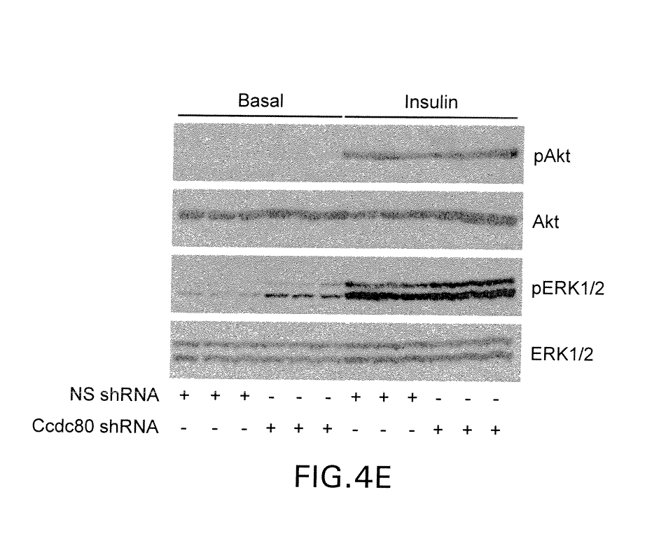

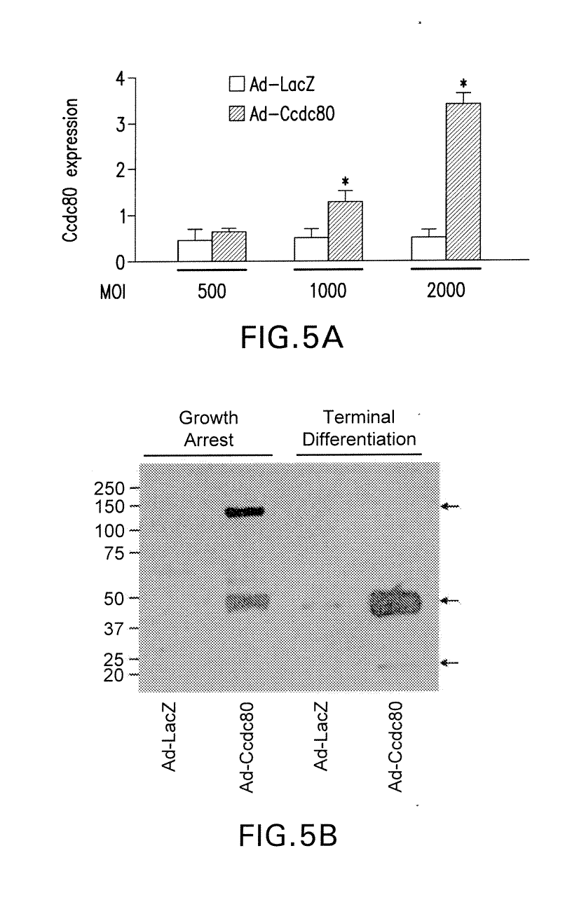

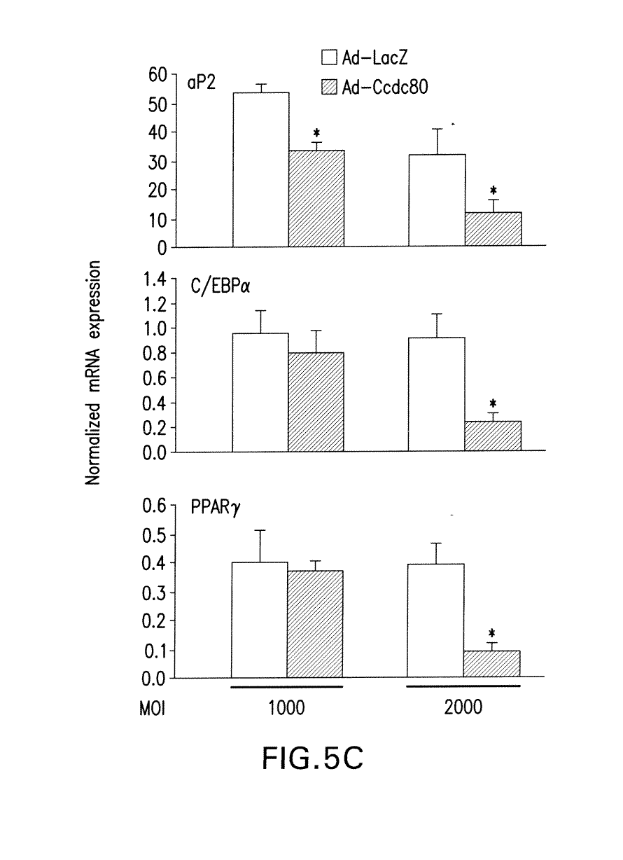

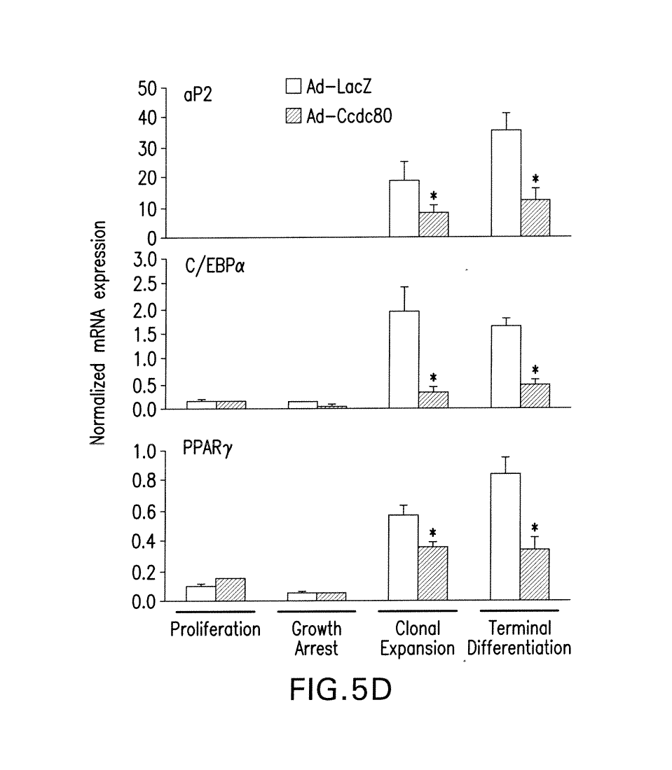

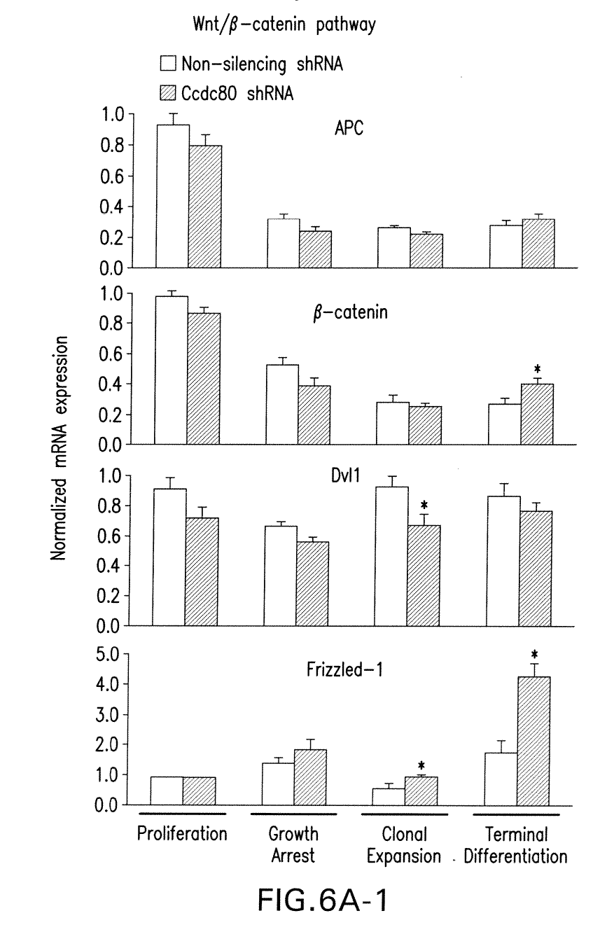

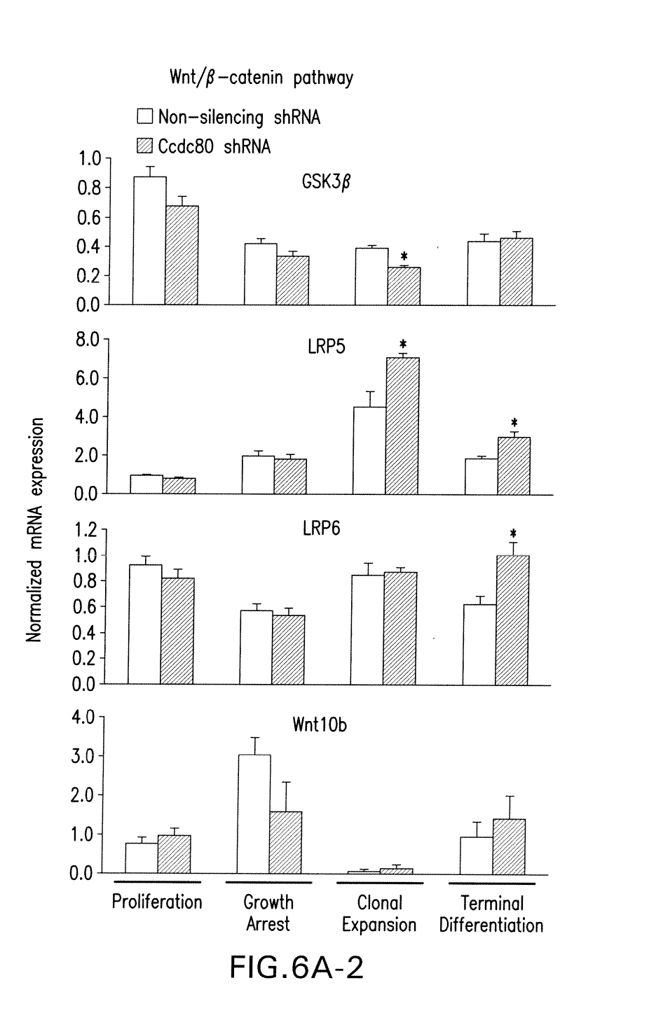

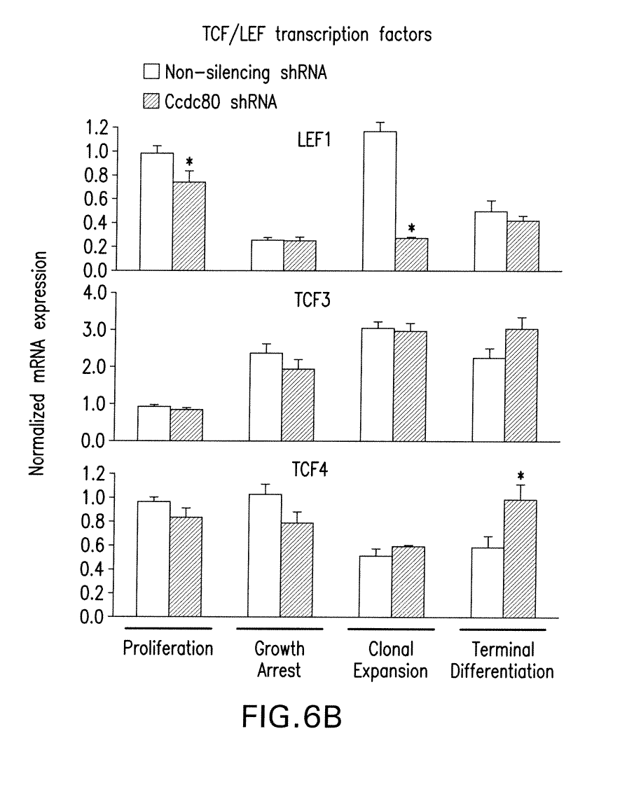

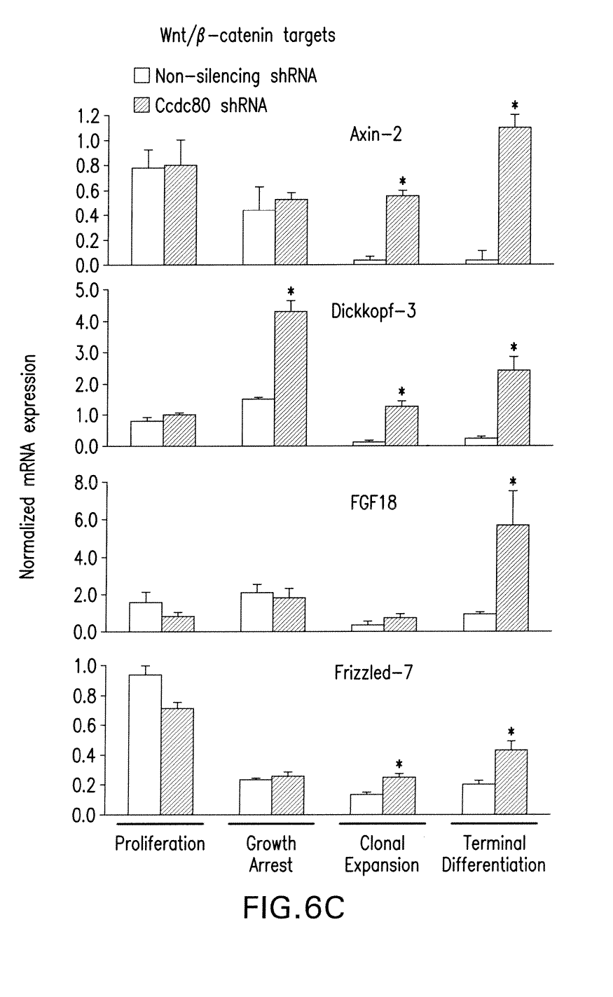

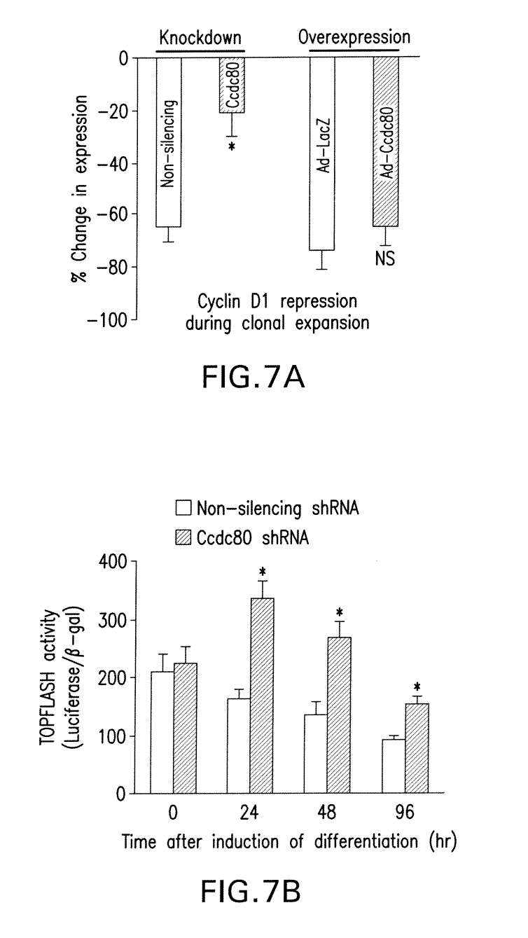

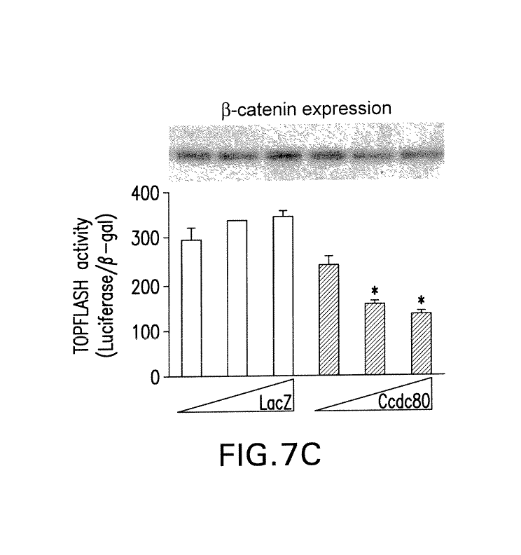

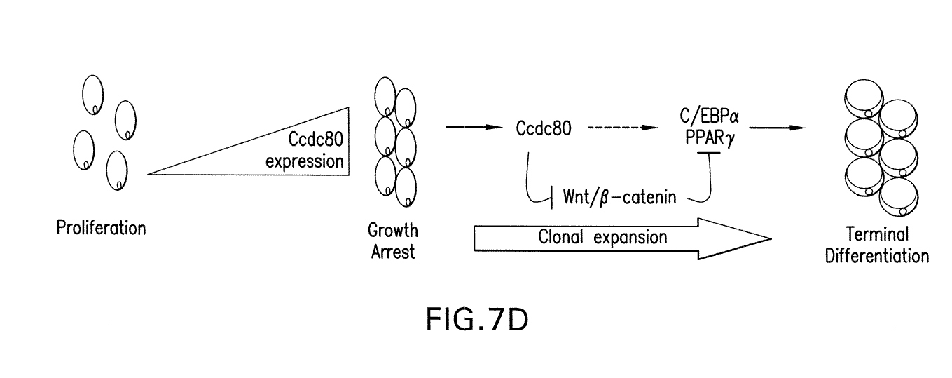

RNA from undifferentiated and differentiated 3T3-L1 adipocytes, tissues from normal, 8- to 12-week-old male C57BL/6J mice, as well as tissues from 10-week-old male ob/ob and age-matched wild-type control mice, was obtained as described (Lake et al., J. Lipid Res 46:2477-2487, 2005). For thiazolidinedione (TZD) treatment, 10-week-old male ob/ob mice were gavaged once per day with 15 mg/kg rosiglitazone or vehicle for 21 days. Primary adipocytes and stromal vascular fraction were prepared from the epididymal adipose tissue of 8- to 12-week-old male C57BL/6J mice as described (Lake et al., J. Lipid Res 46:2477-2487, 2005). Total RNA was extracted using Trizol (Invitrogen) and purified using the RNeasy kit (Qiagen). RNA from human tissues was obtained from Clontech (Mountain View, Calif.). Gene expression profiling was performed using the Mouse Genome 430 2.0 array (Affymetrix) as previously described (Berasi et al., J. Biol. Chem. 281:27167-27177, 2006). Gene expression was also measured by real-time PCR. (n=3-6 mice per group)*p<0.05. Taqman real-time quantitative PCR was performed on a 7900HT fast real-time PCR system (Applied Biosystems) according to the manufacturer's instructions using 18S as an endogenous control as described before (Lake et al., J. Lipid Res 46:2477-2487, 2005). Pre-designed gene-specific primers and probes were obtained from Applied Biosystems. Data shown in FIGS. 1A-E and 1G, FIG. 3 B-D, FIG. 4D, FIG. 5A, C, D, FIG. 6, and FIG. 7A are obtained by real-time PCR; data shown in FIG. 1F, and FIG. 4C are derived from microarray analysis.

### 2. Secretion Experiments

To demonstrate that Ccdc80 is a secreted protein, Applicants cloned the open reading frame of human Ccdc80 (sequence identical to GenBank® Accession No. NM_{—}199511) fused to a C-terminal FLAG tag into the mammalian expression vector pSMED2. The following primers were used:

Forward

(SEQ ID NO:1)

5′-ACGCTGTCGACCACCGCAACCCTCTGCATTCCATCTC-3′;
and
Reverse

(SEQ ID NO:2)

5′-CGTCTAGATTCACTTATCGTCGTCATCCTTGTAATCGTAAGGGTATC
CATGGTGATAACTC-3′.

The Ccdc80-FLAG containing expression vector (pSMED2-Ccdc80-FLAG), as well as a control vector (pSMED2) were transfected into HEK293 T cells. In particular, HEK293T were seeded at a density of 2×10⁶ cells in 10-cm Petri dishes. Cells were transfected with pSmed2 or pSmed2-Ccdc80-FLAG using Fugene6 (Roche). Two days after transfection, cells were placed in serum-free DMEM and medium was collected 24 hr later. Endogenous secretion of Ccdc80 was evaluated in 3T3-L1 preadipocytes and fully differentiated adipocytes. 3T3-L1 cells were rinsed twice with PBS and incubated in serum-free DMEM for 48 hrs before medium was collected. Conditioned media were analyzed by 4-10% SDS-PAGE followed by silver staining or immunological detection with anti-FLAG M2 (293T) or anti-Ccdc80 (3T3-L1) antibodies.

### 3. Antibody Production

Two peptides with 100% sequence homology with mouse and human Ccdc80 were synthesized: KNRVWVISAPHASEGYYR (SEQ ID NO: 5; corresponding to amino acid 148-165 in both mouse and human sequences) and KIDHFQLDNEKPMR (SEQ ID NO:6; corresponding to amino acid 672-685 and 671-684 for human and mouse sequences, respectively). Peptides were conjugated to KLH and injected in a set of two rabbits for 90 days before serum collection (Open Biosystems, Huntsville, Ala.).

### 4. Retroviral Vector and Infection

Retroviral vectors encoding non-silencing and mouse Ccdc80 shRNA were obtained from Open Biosystems. Hairpin sequences were as follows:

control sequence encoding a non-silencing short hairpin RNA:

(SEQ ID NO: 4)

ATCTCGCTTGGGCGAGAGTAAGTGCTGTTGACAGTGAGCGATCTCGCTTG
GGCGAGAGTAAGTAGTGAAGCCACAGATGTACTTACTCTCGCCCAAGCGA
GAGTGCCTACTGCCTCGGA;
and

sequence encoding a short hairpin RNA against Ccdc80 (position 2015-2037):

(SEQ ID NO: 3)

TGCTGTTGACAGTGAGCGCCCTGAGAAGGAGAAGAAGAAATAGTGAAGCC
ACAGATGTATTTCTTCTTCTCCTTCTCAGGTTGCCTACTGCCTCGGA.

Viral packaging was achieved by transfecting 293-VSVG cells with plasmids using Fugene 6. Viral supernatants supplemented with 10 μg/ml polybrene were used to infect 3T3-L1 cells for 48 hrs, followed by selection with 2 μg/ml puromycin.

The mouse Ccdc80 shRNA encoded by SEQ ID NO: 3 was as follows:

(SEQ ID NO: 7)

UGCUGUUGACAGUGAGCGCCCuGAGAAGGAGAAGAAGAAAUAGUGAAGCC
ACAGAUGUAUUUCUUCUUCUCCUUCUCAGGUUGCCUACUGCCUCGGA.

### 5. Adenoviral Vector and Infection

Mouse Ccdc80 cDNA was generated by RT-PCR. Briefly, total RNA was isolated form mouse white adipose tissue using TRIZOL (Invitrogen). cDNAs were synthesized by reverse transcription using random decamers (Ambion). Full-length Ccdc80 was obtained by PCR and ligated into the SalI and XbaI sites of pSmed2. Ccdc80 cDNA was subcloned into pShuttle-CMV followed by linearization with PmeI and electroporation in E. coli BJ5183 cells pre-transformed with the pAdEasy-1 plasmid. Recombinant adenovirus particles encoding mouse Ccdc80 or LacZ (control) were generated according to the manufacturer's instructions (Stratagene).

Infection of 3T3-L1 with adenovirus was performed essentially as previously described (Orlicky and Schaack, J. Lipid Res 42: 460-466, 2001). Briefly, cells were seeded at a density of 1.5×10⁵ cells per well in 6-well plates and grown for 24 hr. Adenovirus were incubated in serum-free DMEM containing 0.5 μg/ml poly-L-lysine (Sigma) for 100 min and the mixture was layered onto PBS-washed cells for 1.5 hr before addition of DMEM containing 20% calf serum. Medium was removed 48 hr later and cells were differentiated as described below.

### 6. Adipocyte Differentiation

3T3-L1 cells were maintained in DMEM containing 20% calf serum in an atmosphere of 10% CO2 at 37° C. Two days post-confluence, cells were induced to differentiate into adipocyte using DMEM containing 10% FBS supplemented with 500 μM 3-isobutyl-1-methylxanthine, 1 μM dexamethasone and 1.7 μM insulin for 4 days, followed by DMEM containing 10% FBS and 0.85 μM insulin for 2 days, then DMEM containing only 10% FBS for an additional 2-4 days. Neutral lipid accumulation in formalin-fixed adipocytes was determined by oil red O staining according to methods well known in the art.

### 7. Insulin Stimulation and Immunoblot Analysis

Differentiated 3T3-L1 adipocytes were deprived of serum for 2 hr before stimulation with 10 nM insulin for 10 min. Cells were rinsed twice in ice-cold PBS and lysed as previously described (Tremblay and Marette, J. Biol. Chem. 276:38052-38060, 2001). Equal amounts of proteins were separated on 4-12% SDS-PAGE and transferred to nitrocellulose membranes. Phosphorylation of Akt (Ser473) and ERK-½ (Thr202/Tyr204) was determined using phospho-specific antibodies (Cell Signaling Technologies).

### 8. Luciferase Reporter Assay

HepG2 cells were seeded at a density of 8×10⁴ cells per well in 24-well plates and grown for 24 hr in antibiotic-free DMEM containing 10% FBS. Cells were transfected with 0.8 μg TOPFLASH and 0.2 μg β-galactosidase reporter plasmids using Lipofectamine 2000 (Invitrogen), rinsed with PBS 4 hr later and infected with adenovirus encoding either GFP or Ccdc80 in opti-MEM. Serum (final concentration: 10% FBS) was added to each well 2 hr after infection and cells were collected 24 hr later. 3T3-L1 cells were seeded at a density of 2.5×10⁵ cells per well in 24-well plates. Cells were transfected with 1 μg TOPFLASH and 0.2 μg β-galactosidase reporter plasmids using Fugene 6 and grown in DMEM containing 20% calf serum until 2 days post-confluency. Cells were collected prior to and 24, 48 and 96 hr after induction of differentiation with insulin, 3-isobutyl-1-methylxanthine and dexamethasone as described above. Luciferase and β-galactosidase activities were measured according to manufacturer's instructions (Promega). Luciferase value was normalized to β-galactosidase activity.

### 9. Statistical Analysis

Results are expressed as mean ±s.e.m. Differences between groups were determined by using unpaired two-tailed student's t-tests and considered to be statistically significant at p<0.05.

### Example 2

### Identification of CCDC80 as a Gene Encoding a Potential New Adipokine

In an attempt to identify new genes encoding adipokines, changes in gene expression occurring in 3T3-L1 adipocytes and mouse white adipose tissue (WAT) during metabolic paradigms were analyzed. To qualify as a potential candidate, the gene should be regulated during 1) adipogenesis, 2) fasting, 3) obesity and 4) insulin sensitization. This transcriptional profiling approach revealed coiled-coil domain containing 80 (Ccdc80) as a gene encoding a potential secreted protein. Ccdc80 encodes a 949 amino acids protein of a predicted molecular weight of 108-kDa. Nucleotide sequence of Ccdc80 open reading frame showed the presence of a putative cleavable signal peptide, multiple nuclear localization signals, three N-linked glycosylation sites, a coiled-coil domain and three internal repeats sharing homology (˜30%) with the fifth domain of Sushi repeats-containing proteins SRPX/SRPX2.

The present inventors searched for secreted proteins that were preferentially expressed in adipose tissue, expressed in primary adipocytes and up-regulated during adipocyte differentiation.

With reference to FIG. 1A, tissue distribution analysis in normal mouse tissues showed that Ccdc80 is highly expressed in WAT with much lower mRNA levels in other tissues. The present inventors also found that Ccdc80 was present in primary adipocytes at significantly higher levels than in the stromal-vascular fraction (FIG. 1F). Furthermore, using an adipogenesis in vitro model, the present inventors found that the conversion of 3T3-L1 cells from preadipocyte to fully differentiated adipocytes was associated with a 5-fold increase in Ccdc80 expression (FIG. 1B).

Ccdc80 gene expression is regulated in vivo. For example, with reference to FIG. 1C, upon fasting, Ccdc80 expression in mouse WAT was reduced by 80% when compared to ad libitum-fed animal. In addition, Ccdc80 mRNA levels were found to be significantly reduced in WAT of obese ob/ob mice relative to their wild-type counterparts (FIG. 1D) and restored to normal level after treatment with the thiazolidinedione (TZD) rosiglitazone (FIG. 1E).

FIG. 1G shows expression of Ccdc80 mRNA in human tissues. As shown in FIG. 1G, Ccdc80 mRNA expression is similar to the mouse in that the highest expression was detected in adipose tissue. Significant, but lower expression of Ccdc80 mRNA was found in uterus, lung, heart, and the thyroid gland. Taking into account the different representation of tissues on human and mouse panels, the tissue distribution of human Ccdc80 is similar to the pattern in mouse and is consistent with Ccdc80 being an adipokine.

The present example demonstrates that Ccdc80 is regulated during adipogenesis and in white adipose tissue during fasting, obesity and after treatment of ob/ob mice with an insulin-sensitizing agent. This provided evidence that Ccdc80 plays a role in the regulation of energy and/or nutrient metabolism.

### Example 3

### Identification of CCDC80 as a Secreted Protein

Prior to the present invention, the question as to whether the Ccdc80 gene encodes a secreted protein yielded contradictory results despite the prediction that it contains an N-terminal signal peptide sequence. For example, one study reported that mouse Ccdc80 is secreted from transiently transfected COS7 cells (Liu, et al., Biochem. Biophys. Res. Commun. 322:497-507, 2004), whereas another showed intracellular expression but not secretion of ectopically expressed human Ccdc80 in COS cells (Bommer, et al., J. Biol. Chem. 280:7962-7975, 2005).

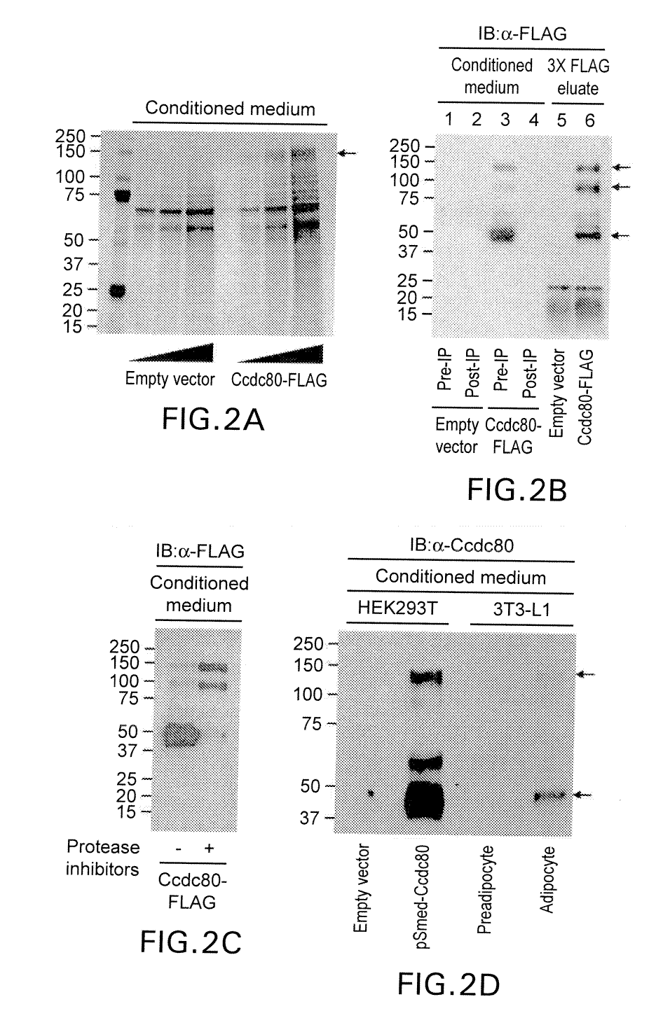

To confirm that Ccdc80 is a secreted protein, human Ccdc80 containing an in-frame C-terminal FLAG epitope was expressed in HEK293T cells. Analysis of serum-free conditioned medium by SDS-PAGE followed by silver staining revealed the presence of a prominent 140-kDa readily detectable in medium from cells expressing Ccdc80 but not from those transfected with an empty vector (FIG. 2A). This band was cut from the gel and mass spectrometry analysis confirmed that this protein was full-length Ccdc80 (63% amino acid coverage; data not shown). The present inventors then analyzed HEK293T supernatants by western blotting using an anti-FLAG antibody and found that Ccdc80 is not only secreted in its full-length form (140-kDa) but also as cleaved fragments of 95-kDa and 50-kDa (FIG. 2B). The observation that the full-length and cleaved fragments of Ccdc80 were effectively depleted from the conditioned medium (lane 3 vs lane 4, FIG. 2B) and recovered after elution with FLAG peptide (lane 3 vs lane 6, FIG. 2B) indicates the presence of an intact C-terminal end. To determine whether processing of Ccdc80 involves an extracellular proteolytic event, HEK293T cells were incubated with a cocktail of protease inhibitors. As shown in FIG. 2C, secretion of the 50-kDa fragment of Ccdc80 was almost totally abrogated by the presence of protease inhibitors with a concomitant increase in the presence of the full-length and 95-kDa cleaved form of Ccdc80 suggesting that high molecular weight forms (140-kDa and 95-kDa) of Ccdc80 serve a substrates for a cell surface-anchored protease.

To examine endogenous Ccdc80 secretion, the present inventors generated a polyclonal antibody using two peptides with 100% sequence homology between mouse and human Ccdc80 (Example 1). Secretion of Ccdc80 was analyzed in conditioned medium obtained from 3T3-L1 preadipocytes and adipocytes and compared to that from HEK293T cells ectopically expressing human Ccdc80. The immunoblot analysis revealed that Ccdc80 is secreted by adipocytes but not preadipocytes (FIG. 2D). In addition, Ccdc80 was secreted from adipocytes as a full-length protein (140-kDa) and as a processed fragment (50-kDa) previously identified in conditioned medium from HEK293T cells (FIG. 2D).

The present example demonstrates that Ccdc80 is a secreted protein, and that it is secreted both as a full-length protein and as cleaved fragments.

### Example 4

### Analysis of CCDC80 Expression During Adipogenesis

Given the results presented in Example 2, which showed that Ccdc80 mRNA levels are upregulated during adipogenesis (FIG. 1B), Ccdc80 gene expression at various phases during the differentiation of 3T3-L1 cells into adipocytes (Schematically illustrated in FIG. 3A) was examined. As shown in FIG. 3B, Ccdc80 is expressed in a biphasic manner with an initial increase in mRNA levels when cells reached growth arrest after proliferation. Then, reduced mRNA levels of Ccdc80 were detected upon induction of differentiation with adipogenic inducers during clonal expansion followed by a higher expression when cells reached terminal differentiation (FIG. 3B).

To determine the temporal relationship between induction of differentiation during clonal expansion and Ccdc80 repression, a time-course was established. A significant reduction in Ccdc80 mRNA levels was observed 8 hrs after the addition of adipogenic inducers (dexamethasone, IBMX and insulin) and was maximal after 24 hr (FIG. 2C). The present inventors then assessed the individual and combined contribution of all adipogenic inducers in the repression of Ccdc80 during clonal expansion. Although each individual component of the cocktail was able to significantly reduce Ccdc80 mRNA levels, a combination of both dexamethasone and IBMX was required to fully repress the expression of Ccdc80 and this was not further enhanced by the addition of insulin (FIG. 3D).

The present example demonstrates that Ccdc80 is expressed in a biphasic manner in 3T3-L1 cells during differentiation.

### Example 5

### Silencing of CCDC80 by RNAI

To examine the role of Ccdc80 in adipocyte function, stable cell lines expressing retroviral vectors encoding either a control (non-silencing) or Ccdc80 shRNA were created. As shown in FIG. 4A, silencing of Ccdc80 by RNA interference reduced the expression of Ccdc80 by 40-50%. Moreover, with reference to FIG. 4B, silencing of Ccdc80 by RNA interference markedly blunted the secretion of the protein. Moreover, lipid accumulation at the end of the adipocyte differentiation protocol was visualized by oil red O staining according to methods well known in the art. The ability of the knockdown cell line to differentiate into adipocytes was inhibited as shown by reduced oil red O staining at the end of the differentiation protocol (data not shown) suggesting that Ccdc80 is required for adipogenesis.

To further explore the mechanisms by which Ccdc80 controls adipocyte differentiation, a gene expression profile of known mediators of adipogenesis, metabolism and signaling was established (FIG. 4C). The expression of C/EBPα and PPARγ was significantly decreased in Ccdc80-knockdown (KD) cells, but not that of C/EBPβ, C/EBPγ, CREB1, E2F1, E2F4 and FOXO1 (FIG. 4C, upper panel). In addition, expression of KLF5, a positive regulator of PPARγ (Oishi et al., Cell Metab. 1:27-39, 2005) and TCF4, a transcription factor involved in β-catenin signaling (van de et al., Cell 111: 241-250, 2002) was significantly increased after silencing of Ccdc80 (FIG. 4C, upper panel). These data suggest that Ccdc80 acts downstream of C/EBPβ/γ and Klf5 and upstream of C/EBPα and PPARγ. It is interesting to note that TCF4 expression was elevated in the knockdown cell line since β-catenin signaling through the TCF transcription factors is known to interfere with induction of C/EBPα and PPARγ expression and to inhibit adipogenesis (Ross et al., Science 289:950-953, 2000). The expression of genes involved in lipid metabolism (aP2, CD36, DGAT½, LIPIN1, LPL and SCD½) was impaired in Ccdc80-KD cells (FIG. 4C, middle panel), an observation consistent with their decreased triglyceride accumulation (as shown by reduced oil red O staining at the end of the differentiation protocol). In addition, expression of the insulin-sensitive glucose transporter GLUT4 was decreased whereas that of the basal glucose transporter GLUT1 was unchanged after Ccdc80 gene silencing. The expression profile of control and knockdown cells showed no obvious difference in the expression of common mediators of the insulin signaling pathway (FIG. 4C, lower panel). Temporal changes in aP2, C/EBPα and PPARγ expression during differentiation revealed that these genes were dramatically induced during clonal expansion and that the magnitude of this increase was severely attenuated in Ccdc80-KD cells, an inhibition maintained throughout terminal differentiation (FIG. 4D).

The present inventors next determined whether the impaired adipogenesis observed in Ccdc80-KD cells was associated with defects in the activation of two mediators of the insulin signaling cascades, Akt and ERK. Insulin-stimulated phosphorylation of both Akt and ERK½, was unaffected by silencing of Ccdc80 with no change in total expression of these proteins (FIG. 4E). Interestingly, Ccdc80-KD cells exhibited elevated basal phosphorylation of ERK½. To examine whether this phosphorylation was responsible for the inhibition of adipogenesis, the present inventors treated these cells with 1 or 10 μM U0126, an inhibitor of MEK, the upstream activator of ERK, and found no reversal of phenotype associated with the inhibition of ERK (data not shown). Furthermore, the present inventors found that treatment of control cells with U0126 during clonal expansion inhibited adipogenesis (data not shown), which is consistent with the requirement of a MEK-dependent phosphorylation of C/EBPα in adipocyte differentiation (Park et al., Mol. Cell. Biol. 24:8671-8680, 2004; Tang et al., Proc. Natl. Acad. Sci. USA 102:9766-9771, 2005).

Since TZD are potent inducers of adipogenesis (Tontonoz et al., Cell 79:1147-1156, 1994) and can up regulate Ccdc80 expression in the white adipose tissue of ob/ob mice (FIG. 1E), the present inventors determined their effect in control and knockdown cells. Cells were differentiated as previously (FIG. 3A) in the presence or absence of rosiglitazone (+/−TZD) at the same time adipogenic inducers were added. In particular, growth-arrested 3T3-L1 cells were differentiated with adipogenic inducers (dexamethasone, IBMX and insulin) in the presence or absence of 100 nM rosiglitazone. Lipid accumulation was visualized by oil red O staining according to methods well known in the art. Treatment with TZD was able to almost fully prevent the defective adipogenesis and lipid accumulation of Ccdc80-KD cells (data not shown). This effect was not associated with restored expression of C/EBPα and PPARγ suggesting that TZD stimulated differentiation of knockdown cells by activating PPARγ, rather than by increasing its expression.

The present example demonstrates that knockdown of Ccdc80 inhibits adipocyte differentiation.

### Example 6

### Analysis of the Effect of Adenovirus-Mediated Overexpression of CCDC80 on Adipogenesis

To further gain insights into the role of Ccdc80 in adipogenesis, the present inventors increased its expression using an adenovirus-mediated overexpression system. Using that strategy, they obtained cells that showed no overexpression (MOI 500; 1-fold) or expression of Ccdc80 at low (MOI 1000; 2-fold) and high (MOI 2000; 5-fold) levels (FIG. 5A). Accordingly, secretion of Ccdc80 from growth-arrested and terminally differentiated cells was increased after adenovirus-mediated overexpression of Ccdc80 (FIG. 5B). The present inventors then determined whether overexpression of Ccdc80 affects the ability of these cells to accumulate lipids. Lipid accumulation in 3T3-L1 cells infected with adenovirus at a MOI of 500, 1000 or 2000 was visualized at the end of the adipocyte differentiation protocol by oil red O staining according to methods well known in the art. It was found that exaggerated (MOI 2000) but not modest (MOI 1000) overexpression of Ccdc80 inhibits adipogenesis as reflected by decreased oil red O staining (data not shown). Consistent with this latter observation, expression of Ccdc80 at high but not low levels severely reduced the expression of aP2, C/EBPα and PPARγ (FIG. 5C). Furthermore, temporal analysis of gene expression changes during differentiation revealed that the induction of adipogenic markers aP2, C/EBPα and PPARγ that normally occurred during clonal expansion was significantly affected by an exaggerated overexpression of Ccdc80 (FIG. 5D). The present inventors finally assessed the ability of TZD treatment to reverse the impaired adipogenesis phenotype associated with massive overexpression of Ccdc80 (MOI 2000). Growth-arrested 3T3-L1 cells infected with adenovirus at a MOI of 2000 were differentiated with adipogenic inducers (dexamethasone, IBMX and insulin) in the presence or absence of rosiglitazone (100 nM). Lipid accumulation was visualized by oil red O staining according to methods well known in the art. As with Ccdc80-KD cells, continuous treatment with rosiglitazone (+TZD) at the beginning of clonal expansion significantly increased the ability of Ccdc80-overexpressing cells to accumulate lipids when compared to cells differentiated using the normal adipogenic cocktail (−TZD) (data not shown).

The present example demonstrates that exaggerated overexpression of Ccdc80 inhibits adipocyte differentiation, whereas modest overexpression does not inhibit adipocyte differentiation.

### Example 7

### Analysis of the Effect CCDC80 Silencing by RNAI on WNT/B-Catenin Signaling

Commitment of growth-arrested 3T3-L1 preadipocytes into adipocytes requires the concomitant down-regulation of Wnt/β-catenin signaling and induction of C/EBPα and PPARγ (Farmer, Cell Metab. 4:263-273, 2006; Rosen and MacDougald, Nat. Rev. Mol. Cell. Biol. 7:885-896, 2006). To explore the possibility that dysregulated β-catenin signaling was responsible for impaired adipogenesis in Ccdc80-KD cells, the present inventors measured the mRNA levels of genes encoding Wnt/β-catenin signaling mediators, transcription factors and target genes using a real-time PCR low-density array. The analysis revealed that expression of target genes of the Wnt/β-catenin signaling pathway (Axin-2, Dickkopf-3, FGF18 and Frizzled-7) was markedly up-regulated by 2- to 35-fold following Ccdc80 silencing by RNAi and occurred in later stages of differentiation (FIG. 6C). Only very little changes in the expression of β-catenin signaling mediators (APC, β-catenin, Dvl-1, Frizzled-1, GSK3 A, LRP5/6 and Wnt10b; FIG. 6A) were associated with the profound up-regulation of Wnt target genes during differentiation (FIG. 6C). Furthermore, the expression of TCF/LEF transcription factors showed down-regulation of LEF1 following clonal expansion and increased TCF4 mRNA levels after terminal differentiation in Ccdc80-KD cells (FIG. 6B). These results suggest that Ccdc80 modulates the transcriptional activity (as reflected by increased expression of target genes, FIG. 6C) rather than the expression per se of components of the Wnt/β-catenin signaling pathway.

The present example demonstrates that knockdown of Ccdc80 increases Wnt/β-catenin signaling.

### Example 8

### Analysis of the Effect of CCDC80 on Efficient Repression of WNT/β-Catenin Signaling During Clonal Expansion

To further explore the possibility that Ccdc80 modulates Wnt/β-catenin signaling, the present inventors measured Cyclin D1 expression during clonal expansion. As shown in FIG. 7A, reduction of Ccdc80 by RNAi severely compromised the ability of adipogenic inducers to repress Cyclin D1 mRNA levels during clonal expansion, whereas adenovirus-mediated overexpression of Ccdc80 had no effect suggesting that endogenous levels of Ccdc80 are sufficient to effectively repress Cyclin D1 expression. The present inventors then examined TCF-mediated transcriptional activity by measuring TOPFLASH reporter activity in 3T3-L1 cells. Upon reaching growth arrest (T=0), cells expressing a non-silencing or Ccdc80 shRNA displayed similar TOPFLASH activity (FIG. 7B). Once differentiation was induced with the adipogenic cocktail, TOPFLASH activity was significantly more elevated in Ccdc80-KD cells throughout clonal expansion (FIG. 7B). Conversely, overexpression of Ccdc80 in HepG2 cells, which express a stabilized form of β-catenin (de La Coste et al., Proc. Natl. Acad. Sci. USA 95: 8847-8851, 1998), resulted in a dose-dependent inhibition of TOPFLASH reporter activity without affecting β-catenin protein expression (FIG. 7C). These data indicates that the elevated expression of Ccdc80 during growth arrest is necessary for the efficient repression of Wnt/β-catenin signaling during clonal expansion and further suggests its requirement for C/EBPα and PPARγ induction and normal lipid accumulation during terminal differentiation of adipocytes (FIG. 7D).

The present example demonstrates that Ccdc80 is required for the efficient repression of Wnt/β-catenin signaling during adipogenesis.

What is claimed is:

1. A method of modulating adipogenesis in a cell, the method comprising:
contacting the cell with an agent that modulates the expression or activity of the Ccdc80 gene or Ccdc80 protein.

2. The method of claim 1, wherein the cell is an adipocytic cell selected from the group consisting of a pre-adipocyte, adipocyte, mesenchymal stem cell, embryonic stem cell and embryonic fibroblast.

3. The method of claim 1, wherein the agent is a compound, a protein, a peptide, an antibody, an aptamer, or a polynucleotide.

4. The method of claim 1, wherein the agent increases Ccdc80 gene expression or Ccdc80 protein expression or activity.

5. The method of claim 1, wherein the agent prevents or reduces Ccdc80 gene expression or Ccdc80 protein expression or activity.

6. The method of claim 5, wherein the agent prevents or reduces at least one of Ccdc80 gene transcription and translation of Ccdc80 messenger ribonucleic acid (mRNA)

7. The method of claim 5, wherein the agent is a polynucleotide.

8. The method of claim 1, wherein the agent directly modulates the expression or activity of the Ccdc80 gene or Ccdc80 protein.

9. The method of claim 7, wherein the polynucleotide is ribonucleic acid (RNA).

10. The method of claim 9, wherein the polynucleotide is selected from the group consisting of a double stranded RNA (dsRNA), a ribozyme or an antisense oligonucleotide.

11. The method of claim 9, wherein the polynucleotide is a short hairpin RNA (shRNA) that comprises the nucleic acid sequence of SEQ ID NO: 7.

12. The method of claim 7, wherein the polynucleotide is deoxyribonucleic acid (DNA).

13. The method of claim 7, wherein the polynucleotide is linked to a peptide or antibody which binds to at least one cell surface receptor or antigen of the cell.

14. The method of claim 5, wherein the agent is an antibody against Ccdc80 protein.

15. A method of modulating Wnt/b-catenin signaling in a cell, the method comprising:
contacting the cell with an agent that modulates the expression or activity of the Ccdc80 gene or Ccdc80 protein.

16. The method of claim 15, wherein the agent is selected from the group consisting of a compound, a protein, a peptide, an antibody, an aptamer, or a polynucleotide.

17. The method of claim 16 wherein the agent directly modulates the expression or activity of the Ccdc80 gene or Ccdc80 protein.

18. The method of claim 16, wherein the agent is a short hairpin RNA (shRNA).

19. The method of claim 18, wherein the shRNA comprises a nucleic acid sequence that hybridizes under high stringency conditions to a Ccdc80 gene sequence of SEQ ID NO: 3.

20. The method of claim 19, wherein the shRNA comprises the nucleic acid sequence of SEQ ID NO: 7.

21. A method of treating a condition selected from obesity, insulin resistance, or type 2 diabetes comprising:
administering to a subject in need thereof an agent that modulates the expression or activity of the Ccdc80 gene or Ccdc80 protein.

22. The method of claim 21, wherein the condition treated is obesity.

23. The method of claim 22, wherein the agent is administered to a subject having excess body weight as a method of cosmetic treatment of obesity.

24. The method of claim 22, wherein the agent administered to treat obesity increases Ccdc80 gene expression or Ccdc80 protein expression or activity.

25. The method of claim 22, wherein the agent administered to treat obesity prevents or reduces Ccdc80 gene expression or Ccdc80 protein expression or activity.

26. The method of claim 25, wherein the agent prevents or reduces at least one of Ccdc80 gene transcription or translation of Ccdc80 messenger ribonucleic acid (mRNA).

27. The method of claim 21, wherein the agent directly modulates the expression or activity of the Ccdc80 gene or Ccdc80 protein.

28. The method of claim 26, wherein the agent is a double stranded RNA (dsRNA), a ribozyme, or an antisense oligonucleotide.

29. The method of claim 26, wherein the agent is a short hairpin RNA (shRNA) that comprises a nucleic acid sequence that hybridizes under high stringency conditions to a Ccdc80 gene sequence of SEQ ID NO: 3.

30. The method of claim 29, wherein the shRNA comprises the nucleic acid sequence of SEQ ID NO: 7.

31. A method of screening for an agent that modulates adipogenesis, the method comprising:
providing a cell that expresses the Ccdc80 gene;
contacting the cell with a candidate agent; and
evaluating the ability of the candidate agent to modulate the expression or activity of the Ccdc80 gene or Ccdc80 protein in the cell, wherein a candidate agent that modulates said expression or activity is an agent that modulates adipogenesis.
